(19) United States
(12) Patent Application Publication
(10) Pub. No.: US 2005/0125160 A1
(43) Pub. Date: Jun. 9, 2005

(54) Modifying the control of gene expression behavior by the deletion of connectrons and by the design and addition of synthetic connectrons in prokaryotic, archea and eukaryotic genomes

(76) Inventor: Richard J. Feldmann, Derwood, MD (US)

Correspondence Address: Law Office of Jim Zegeer, Suite 108, 801 North Pitt Street, Alexandria, VA, 22314, US

(21) Appl. No.: 10/609,383

(22) Filed: Jul. 1, 2003

Related U.S. Application Data
(63) Continuation-in-part of application No. 09/866,925, filed on May 30, 2001.
(60) Provisional application No. 60/393,558, filed on Jul. 5, 2002.

(51) Int. Cl. G06F 19/00; G01N 33/48; G01N 33/50

(52) U.S. Cl. 702/20

(57) ABSTRACT

The connectron structure of a genome determines sets of four DNA sequences of minimum length of 15-bases (C1 and C2 which are in the 3′UTR of a gene, T1 which is on the 5′-side and T2 which is on the 3′-side of a set of genes). The connectron state of a genome provides information that makes it possible to decide where and how connectrons can be deleted from the native genome or added to the native genome to modify genomic gene expression behavior.

### REFERENCE TO RELATED APPLICATION

The present application includes the subject of Provisional Application Ser. No. 60/393,558 filed Jul. 5, 2002

The present application is a continuation in part of U.S. patent application Ser. No. 09/866,925 filed May 30, 2001 entitled ALGORITHMIC DETERMINATION OF FLANKING DNA SEQUENCES THAT CONTROL THE EXPRESSION OF SETS OF GENES IN PROKARYOTIC, ARCHEA AND EUKARYOTIC GENOMES

The present application is also related to U.S. patent application Ser. No. 10/339,666 filed Jan. 10, 2003 entitled SIMULATION OF GENE EXPRESSION CONTROL USING CONNECTRONS, INTERFERENCE RNAS (IRNAS) AND SMALL TEMPORAL RNAS (STRNAS) IN PROKARYOTIC, ARCHEA AND EUKARYOTIC GENOMES

The present application is also related to U.S. patent application Ser. No. 10/364,516 filed Feb. 12, 2003 entitled DETERMINATION OF FLANKING DNA SEQUENCES THAT CONTROL THE EXPRESSION OF SETS OF GENES IN THE ESCHERICHIA COLI K-12 MG1655 COMPLETE GENOME

The present application is also related to U.S. patent application Ser. No. 10/364,412 filed Feb. 12, 2003 entitled DETERMINATION OF FLANKING DNA SEQUENCES THAT CONTROL THE EXPRESSION OF SETS OF GENES IN THE SACCHAROMYCES CEREVISIAE COMPLETE GENOME

The present application is also related to U.S. patent application Ser. No. 10/299,056 filed Nov. 19, 2002 entitled DETERMINATION OF FLANKING DNA SEQUENCES THAT CONTROL THE EXPRESSION OF SETS OF GENES IN THE HALOBACTERIUM SP. NRC-1 COMPLETE GENOME

The present application is also related to U.S. patent application Ser. No. 10/367,832 filed Feb. 19, 2003 entitled DETERMINATION OF FLANKING DNA SEQUENCES THAT CONTROL THE EXPRESSION OF SETS OF GENES IN THE PSEUDOMONAS AERUGINOSA PA01, COMPLETE GENOME

The present application is also related to U.S. patent application Ser. No. 10/287,818 filed Nov. 5, 2002 entitled DETERMINATION OF FLANKING DNA SEQUENCES THAT CONTROL THE EXPRESSION OF SETS OF GENES IN THE VIBRIO CHOLERAE CHROMOSOME I, COMPLETE CHROMOSOME

The present application is also related to U.S. patent application Ser. No. 10/305,275 filed Nov. 27, 2002 entitled DETERMINATION OF FLANKING DNA SEQUENCES THAT CONTROL THE EXPRESSION OF SETS OF GENES IN THE AEROPYRUM PERNIX K1 COMPLETE GENOME

### INTRODUCTION

The connectron structure of a genome determines sets of four DNA sequences of minimum length of 15-bases (C1 and C2 which are in the 3′UTR of a gene, Ti which is on the 5′-side and T2 which is on the 3′-side of a set of genes). The connectrons in a genome control the expression of sets of genes. Each native genome has a particular set of connectrons that control the expression of sets of genes in many different ways. The gene expression behavior can be changed by deleting existing connectrons and by adding new connectrons that have been designed to achieve specific new gene expression patterns. This patent application describes the invention of methods for deleting connectrons as well as the invention of four classes of synthetic and designed connectrons that can be used to modify the connectron behavior of a genome. In addition this application describes the invention of four methods that implement synthetic connectrons with differing proportions of novelty.

### DEFINITIONS

Double Stranded DNA

Watson and Crick showed in 1953 that DNA naturally forms a double-stranded helix. A typical double stranded sequence is

5′-TAGAGGACTACCAC-3′
3′-ATCTCCTCATGGTG-5′

Hydrogen Bond

The force between a hydrogen atom and another heavier atom such as Oxygen (O), Nitrogen (N), Phosphorus (P), or Sulfur (S).

Positive Strand

The positive strand is normally represented 5′ to 3′ running left to right as in

5′-TAGAGGACTACCAC-3′

Negative Strand

The negative strand is normally represented 5′ to 3′ running right to left as in

3′-ATCTCCTCATGGTG-5′

Single Stranded RNA

Either the positive or the negative strand of the double-stranded DNA can be transcribed by the polymerase. In RNA U replaces T.

| | |
|---|---|
| RNA of positive | 5′-UAGAGGAGUACCAC-3′ |
| strand sequence | |
| RNA of negative | 5′-GUGGUACUCCUCUA-3′ |
| strand sequence | |

Antisense RNA

The antisense strand of any RNA sequence is the complement sequence

| | |
|---|---|
| RNA sequence | 5′-UAGAGGAGUACCAC-3′ |
| Antisense RNA sequence | 3′-AUCUCCUCAUGGUG-5′ |

Triple Strand Helix

The RNA sequence of a RNA/DNA triple-strand complex is the same as the positive strand of the DNA

| | |
|---|---|
| DNA positive strand | 5′-TAGAGGAGTACCAC-3′ |
| DNA negative strand | 3′-ATCTCCTCATGGTG-5′ |
| RNA strand | 5′-UAGAGGAGUACCAC-3′ |

Promoter

Any region of DNA, that binds proteins which engage the polymerase transcription mechanism.

TATA Box

A region near the 3′ end of a promoter with the sequence TATA.

mRNA

The RNA produced from the DNA by the polymerase as a result of transcription

Start of Transcription

The 3′ end of a promoter where the polymerase mechanism begins to transcribe DNA into mRNA.

Exon

Any region of mRNA which is used to code for proteins

Intron

Any region of mRNA lying between two exons which is not used to code for proteins. The introns are edited out of the initial RNA transcript to form the mature mRNA.

3′ UTR

The untranslated 3′ end of an mRNA is beyond the end of the last exon. A stop codon in the mRNA causes the ribosome to stop the translation of mRNA into protein.

End of Translation

The 3′ end of the 3′-most exon.

Translated Region

Any collection of exons and introns.

Gene

Any DNA region that codes for a protein. Introns do not occur in prokaryotic genes and they sometime fail to occur in eukaryotic genes. A typical model of a gene is

|<-----Promoter------>|

|<−TATA Box−>|

|<−Beginning of Translation
| [symbol] ------------Translated Region---------------->|

End of Translation−>|

|<−Exon−>|<−Intron−>|<−Exon−>|<−Intron−>|<−Exon−>|<−3′UTR−>|
+ strand -----------------------------------------------------------
− strand -----------------------------------------------------------
|<---------------------------------Gene----------------------------->|

Positive Strand Gene

Any gene in which the features run 5′ to 3′ on the positive strand

Negative Strand Gene

Any gene in which the features run 5′ to 3′ on the negative strand

C1 Sequence

Any positive or negative strand DNA sequence of 15 bases or more. The C2 sequence must occur in the same chromosome as the C1 sequence.

C2 Sequence

Any positive or negative strand DNA sequence of 15 bases or more. The C1 sequence must occur in the same chromosome as the C2 sequence.

C1/C2

Any positive or negative strand DNA sequence of 40 or more bases such that the C1 sequence is adjacent to the C2 sequence

T1 Sequence

Any positive or negative strand DNA sequence of 15 bases or more that is on the same chromosome as the T2 sequence. The T1 and T2 sequences must be between about 1 kb and 105 kb apart.

T2 Sequence

Any positive or negative strand DNA sequence of 15 bases or more that is on the same chromosome as the T1 sequence. The T2 and T1 sequences must be between about 1 kb and 105 kb apart.

Last Exon Gap or Gap-Distance

The number of bases between the end of transcription and the beginning of the C1/C2 sequence. In prokaryotes and single-celled eukaryotes this gap can range from no bases to 500 bases. In multi-celled eukaryotes the gap can be as large as 10,000 bases.

Poly-Adenylation Signal

A number of Adenosine (A) bases are added to the mRNA at the end of the 3′UTR.

Possible Connectron

Any set of T1, T2 and C1/C2 sequences such that the C1 sequence is identical to the T1 sequence and the C2 sequence is identical to the T2 sequence. The promoter of some gene causes the mRNA of the gene to be expressed. The mRNA is edited to eliminate the introns. The whole mRNA including the 3′UTR can move about in the cell or the nucleus of the cell. The C1/C2 RNA that is part of the 3′UTR moves to the T1 and T2 DNA sequences. A triple-stranded complex of the DNA and the RNA forms such that the C1 sequence forms hydrogen bonds with the T1 sequence and the C2 sequence forms hydrogen bonds with the T2 sequence. Because the C1 sequence is adjacent to the C2 sequence, the T1 sequence is brought physically close to the T2 sequence. This produces a loop of between about 1 kb and 105 kb in the DNA. Histone proteins reduce the length of the DNA by binding 200 bases. Histone/DNA complexes form six-fold symmetry chromatin assemblies. The diameter of the chromatin assemblies is approximately 30 nm.

Real Connectron

Any Possible Connectron which is within the Gap-Distance of some gene

Homologous connectron

The T1 sequence and the T2 sequence are on the same chromosome as the C1/C2 sequence

Heterologous Connectron

The T1 sequence and the T2 sequence are on a chromosome different from chromosome of the C1/C2 sequence

Permanent Connectron

Any C1/C2 sequence, which is 3′ UTR to some gene that is not surrounded by any T1 and T2 sequence pairs

Transient Connectron

Any C1/C2 sequence, which is 3′ UTR to some gene that is surrounded by one or more T1 and T2 sequence pairs

One-Shot or Self-Limiting Connectron

Any C1/C2 sequence which is 3′UTR to some gene that is surrounded by the T1 and T2 sequences such that C1=T1 and C2=T2

Geneless Connectron

Any C1/C2 sequence that is not 3′UTR to some gene but is surrounded by some T1 and T2. A promoter may lie 5′ to the C1/C2 sequence.

Bi-Directionality of Connectron Excitation

A C1/C2 short loop on one strand selects a T1-T2 long loop pair on the same or the opposite strand. The C1/C2 short loop has a complementary C1′/C2′ sequence on the opposite strand. Similarly the T1-T2 long loop pair has a complementary long loop pair T1′-T2′. Wherever a C1/C2, T1-T2 tetrad exists there is a complementary C1′/C2′, T1′-T2′ tetrad. The C1/C2 short loop can be transcribed as a 3′UTR to a gene on the same strand. The C1′/C2′ short loop which is on the strand opposite to the C1/C2 short loop can also can be transcribed as a 3′UTR to a gene on the same strand. There are four possible models of action

| | | |
|---|---|---|
| T1 | T2 | gene - C1/C2 |

+ strand ---------------------------------------------------------------
− strand ---------------------------------------------------------------

| | |
|---|---|
| T1 | T2 |

+ strand ----------------------------------------------------------------
− strand ----------------------------------------------------------------

C2/C1 - gene

+ strand ----------------------------------------------------------------
− strand ----------------------------------------------------------------

| | | |
|---|---|---|
| T2′ | T1′ | C2′/C1′ - gene |

gene - C1′ /C2′

+ strand ----------------------------------------------------------------
− strand ----------------------------------------------------------------

| | |
|---|---|
| T2′ | T1′ |

Of course, the short loops and the long loops do not have to be on the same chromosome.

Hierarchy of Connectron Action

When a C1/C2 is expressed it forms a T1-T2 loop by forming a connectron. The C1/C2 sequence does not have to be on the same chromosome as the T1 and T2 sequences. This provides a way of causing interaction between chromosomes. When the T1-T2 loop forms, any genes in that loop region which had been expressing C1/C2 sequences in their 3′UTRs, now cease expressing the C1/C2 sequences. The connectrons formed by these C1/C2 sequences will cease to exist after some time thus opening up the genes inside the respective T1-T2 loops to expression. The hierarchy of connectron action is alternates between repression and expression. The connectron hierarchies can be of any depth.

One-to-Many Connectron Action

One C1/C2 sequence can form connectrons in many different places on many different chromosomes. The only requirement is that C1=T1 and C2=T2. This makes it possible for one expression event to control the expression of many genes on different chromosomes.

Many-to-One Connectron Action

C1/C2s that come from many different places on many different chromosomes can form a connectron for a specific T1-T2 sequence pair. The only requirement is that C1=T1 and C2=T2. This makes it possible for many different expression events to control the expression of one set of genes on a particular chromosome.

Many-to-Many Connectron Action

The arrangement of C1/C2s and T1-T2s across chromosomes can form a complex web of gene expression control relationships.

Percentage of the Genome Regulated by Connectrons

Since the connectrons for a sequenced genome can be calculated, the percentage of the genome that is open to connectron regulation can be known.

Emergent Property

The network of connectrons in any genome emerges from a knowledge of the complete DNA sequence of the genome. Because both the C1/C2 sequences and the T1-T2 sequences can be any place in the genome, the whole genomic sequence must be known before all the connectrons can be determined.

Paradigm Shift

For the past fifty years since the discovery by Watson and Crick of the double-helical nature of DNA, the reigning paradigm for scientific discovery has been the study of one gene and its effects on the behavior of a cell. The advent of genomic sequencing and this invention of connectrons that emerge from the whole genome will produce a shift in the way scientists view biological systems and the way they formulate and execute experiments. The many-to-many relationships between the connectrons mean that there are many ways in which the expression of a set of genes can be modulated. The multiplicity of control pathways produces a system stability that makes it possible for biological systems to be stable for long periods of evolutionary time. The thinking that goes into formulating scientific experiments will have to change to accommodate the changes in understanding that will be induced by the application and extension of this patent application.

Sequence Element

The double-stranded sequence of either a C1, a C2, a T1 or a T2 of some connectron.

Native Sequence Element

Any sequence element of a connectron which is a constitutive part of the native genome.

Native Connectron

Any connectron in which all four (i.e. the C1, C2, T1 and T2 sequence) sequence elements are constitutive parts of the native genome.

Synthetic Sequence Element

Any sequence element that has been added into a genome at some particular place. The actual sequence of bases may occur in one or more places in the genome. The synthetic aspect is that an instance has been placed into the genome at a specific place by design.

Copied Sequence Element

Any sequence element that has been copied from one place in a genome to another place in that genome.

Unique Sequence Element

Any double-stranded DNA sequence element for which there is only one pair of instances. Such a pair of instances can be used to implement the C1 and T1 synthetic sequence elements of a connectron or the C2 and T2 synthetic sequence elements of a connectron.

Extracted Sequence

Any sequence element that is moved from one place in a genome to another place in the same genome. The moved sequence element can be unique or non-unique.

Synthetic Connectron

Any connectron in which one or more of the sequence elements in a native connectron are substituted by synthetic sequence elements.

Designed Connectron

Any connectron that does not exist in the native genome.

RNA-Based Connectron

Any connectron in which the C1 and C2 elements are generated as RNA in the 3′UTR of some gene.

DBP

A multi-domain zinc-finger DNA Binding Protein that binds to a specific DNA double-stranded sequence.

Protein-Based Synthetic Connectron

Any synthetic connectron in which the C1 and C2 elements are generated by a DBP.

PNA

A Peptide Nucleic Acid is a molecular construct patented and supplied by Boehringer-Mannheim. The A, T, G, C base portions of are the same as an RNA but the ribose ring has been replaced by a peptide-like configuration of atoms.

PNA-Based Synthetic Connectron

Any synthetic connectron in which the C1 and C2 elements are generated by a sequence of Peptide Nucleic Acid (PNA) bases.

Generalized Synthetic Connectron

Any synthetic connectron in which the C1 and C2 elements are generated by two linked double-strand DNA binding elements.

Individual Connectron State

A binary representation of whether the C1-C2 elements are bound to the cognate T1 and T2 elements. A 0 means that the connectron is not formed. A 1 means that the connectron is formed and hence the expression of the genes between the T1 and T2 elements are turned off.

Genomic Connectron State

The collection of all the individual native and synthetic connectron states in a genome. The individual connectron states are ordered along each chromosome and plasmid by the position of the T1 element.

Connectron Simulation

The temporal transitions of the genomic connectron states as the individual connectron states interact with each other.

### Content of the CD

The Sequence Listing Part is incorporated herein by reference. The material contained on this compact disc attached hereto was created on Jun. 30, 2003,

synthetic.sequences has 13,770 bytes

### BRIEF DESCRIPTION OF THE OBJECTS OF THE INVENTION

The basic methods patent application provides the methods for determining the structure of the native connectrons in a variety of genomes. Since the original conceptualization of connectrons followed from the work that we did on the design of zinc-finger DNA Binding Proteins (DBPs), U.S. Pat. No. 6,205,404, it has always been the applicant's intention to use these designed proteins to modify genomic connectron behavior.

An object of this invention is to provide a method for utilizing the genomic simulation of connectron behavior to facilitate the optimization of design decisions related to deleting connectrons from a genome or adding connectrons to a genome.

An object of this invention is to provide a method for utilizing the genomic simulation by computer of connectron behavior to facilitate the optimization by computer of design decisions related to deleting connectrons from a genome or adding connectrons to a genome.

An object of this invention is to provide a method for modifying the connectron behavior of a genome by deleting and adding connectrons.

An object of the invention is to provide a method for creating new connectrons in a genome by copying one or more double-stranded DNA sequence elements from one place in the genome to another place in the genome or by extracting a double-stranded DNA sequence element from one place in the genome and moving it to another place in the genome or by introducing a new unique pair of double-stranded DNA sequence elements into the genome at specific places.

An object of the invention if to provide a method for creating new connectrons in a genome by copying, extracting or adding unique DNA elements in addition to using either DBPs, PNAs or a linked pair of generalized DNA binding elements to implement the C1 and C2 elements of the new connectron.

### DESCRIPTION OF THE TABLES

Table 1

The 10 possible ways of deleting a connectron

Table 2

The 15 possible synthetic connectrons defined by combinations of native and synthetic elements

Table 3

The 4 possible synthetic connectrons defined by combinations of native and synthetic DNA elements and synthetic DBP elements

Table 4

The 4 possible synthetic connectrons defined by combinations of native and synthetic DNA elements and synthetic PNA elements

Table 5

The 4 possible synthetic connectrons defined by combinations of native and synthetic DNA elements and a pair of linked double-strand DNA binding elements

Table 6

The 2 possible ways of implementing a single synthetic element in a synthetic connectron

Table 7

The 14 possible ways of implementing a pair of synthetic elements in a synthetic connectron

Table 8

The 41 possible ways of implementing three synthetic elements in a synthetic connectron

Table 9

The 25 possible ways of implementing four synthetic elements in a synthetic connectron

### DESCRIPTION OF THE INVENTION

The simulation of the connectron behavior of a genome provides a powerful way of understanding how individual connectrons interact with each other. From the more than 100 genomes whose connectron structure we have determined, it is clear that a given C1-C2 may generate many different connectrons—the one-to-many connectron action mode. Similarly it is clear that a given connectron may be generated by the expression of many different instances of the C1-C2 signal—the many-to-one connectron action mode. The temporal interaction of these one-to-many and many-to-one modes of connectron control of gene expression can best be understood by doing a genomic connectron simulation and looking at the results. This is especially important when determining when and where to delete connectrons from a native genome or add new connectrons to a native genome. Genomic connectron simulation is the subject of a related patent application.

Although there are instances in different genomes of a C1-C2 generating only one connectron, much more frequently a given C1-C2 will generate several connectrons. When only one connectron is generated by a given C1-C2, a decision to delete the connectron or to add another connectron using the same or different C1 or C2 elements will have no side effects as long as the T1 and T2 elements are only used by this connectron. This is the relative infrequent example of one-to-one. Much more frequently, deleting or changing a given C1 or C2 may have multiple consequences and serious behavioral side effects. Modification of genomic connectron control of gene expression will inevitably involve compromises, design decisions, and—worst of all—patch-ups. Consider an example in which a given C1-C2 generates two connectrons. For some reason we desire to delete one of the two connectrons. A patch-up can be achieved by finding some other way to generate the second connectron. This could be achieved by adding one or more synthetic connectron elements to generate the second connectron as a synthetic connectron.

If there are no side effects of a design decision then that is the optimum. If there are side effects, the genomic simulation of connectron behavior makes it possible to evaluate the genomic behavioral consequences of the design and implementation decisions.

The deletion of a connectron as shown in table 1 can occur in 10 different ways ranging from deleting or modifying just one sequence element to deleting or modifying all four sequence elements. Anything that destroys the sequence recognition of the T1 by the C1 or the T2 by the C2 will have the effect of deleting the connectron.

Any or all of the four elements (C1, C2, T1 and T2) of a native connectron can be replaced with synthetic elements to form a synthetic connectron. Table 2 gives the 15 possible types of synthetic connectrons. A designed connectron can be introduced into a genome to either (1) control the expression of a set of genes already controlled by other native connectrons, or (2) control the expression of a newly selected set of genes. Using DNA sequences, a designed connectron be made from any of the 15 combinations of native and synthetic elements in Table 2. If the C1 and C2 elements of a connectron are generated by a zinc-finger DBP then the 4 combinations of synthetic and designed connectrons in table 3 are possible. If the C1 and C2 elements of a connectron are generated by a PNA then the 4 combinations of synthetic and designed connectrons in table 4 are possible. If the C1 and C2 elements of a connectron are generated by a linked pair of DNA binding elements then the 4 combinations of synthetic and designed connectrons in table 5 are possible. The DNA binding elements G1 and G2 can be proteins or any other molecular material that recognizes the DNA sequences of the T1 and T2 elements.

The implementation of the synthetic elements of a designed connectron can be achieved in several different ways. In table 2 from one to four of the sequence elements of a connectron can be substituted by a synthetic sequence element. There are three different ways of creating a synthetic sequence element. An existing sequence element somewhere in the genome can be copied into a new location. An existing sequence element can be extracted from one location in the genome and placed in another position in the genome. A new pair of sequence elements not otherwise present in the genome can be created. One instance of this new, unique sequence element must be a C1 or a C2. If the first instance is a C1 then the second instance of this new, unique sequence element must be a T1. Similarly, if the first instance is a C2 then the second instance of this new, unique sequence element must be a T2. Table 6 shows that there are only two ways of generating a synthetic connectron when only one synthetic sequence element is used. Implementing a new, unique sequence element is not possible because these sequence elements must come in pairs. Table 7 shows that there are 14 ways of implementing a pair of synthetic sequence elements to form a synthetic connectron. Table 8 shows that there are 41 ways of implementing three synthetic sequence elements to form a synthetic connectron. Table 9 shows that there are 25 ways of implementing four synthetic sequence elements to form a synthetic connectron.

### EXAMPLES

Tables 1 thru 9 provide a large combinatorial number of ways of deleting connectrons from a genome and of adding connectrons to a genome. We give one example of deleting one connectron from a single genome and five examples of a connectron to four different genomes. It is clear that many other variations would be obvious to someone skilled in the art.

### Deletion of a Transient Connectron from One Chromosome to Itself

Connectron 13 is an example of a transient connectron in the S. cerevisiae genome. As per table 1, this connectron can be deleted by either deleting or modifying any one or all four of the connectron elements. The C1 and the C2 identifiers are 36 while the T1 identifier is 39 and the T2 identifier is 111. When this connectron is deleted, all the properties stated below disappear.

The original connectron 13 is described as

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 13 | 1 | 36 | 36 | 1 | 39 | 111 | transient |

The C1/C2 source of the transient connectron 13 is represented in table 1 as

| Type | Num | Jobno | Chr | Start | Stop | Length | GeneName |
|---|---|---|---|---|---|---|---|
| CPT | 36 | 1 | 1 | 12.572 | 12.788 | .217 | OS−> \|\|\|\|\|\|\|\|\|\|\|\|\|\|\|\|\|\| |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB).

The T1-T2 target of the transient connectron 13 as shown in table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TP | 39 | 1 | 1 | 12.572 | 12.807 | .236 | *−+++++++++++++++++++* |
| TN | 40 | 8 | 1 | 12.908 | 12.964 | .057 | *−++++++++++++++++++++* |
| CNT | 41 | 1 | 1 | 12.908 | 12.964 | .057 | −−> \|\|\|\|\|\|\|\|\|\|\|\|\|\|\|\|\|\|\|\|\| |
| CPT | 42 | 1 | 1 | 12.908 | 12.964 | .057 | −−> \|\|\|\|\|\|\|\|\|\|\|\|\|\|\|\|\|\|\|\|\| |
| TP | 43 | 1 | 1 | 12.922 | 12.964 | .043 | *−+++++++++++++++++++++* |
| CNT | 44 | 1 | 1 | 12.966 | 12.986 | .021 | −−> \|\|\|\|\|\|\|\|\|\|\|\|\|\|\|\|\|\|\|\|\|\| |
| CPT | 45 | 8 | 1 | 12.966 | 12.986 | .021 | −−> \|\|\|\|\|\|\|\|\|\|\|\|\|\|\|\|\|\|\|\|\|\| |
| TP | 46 | 1 | 1 | 12.966 | 12.986 | .021 | *−++++++++++++++++++++++* |
| TN | 47 | 1 | 1 | 13.027 | 13.159 | .133 | *−+++++++++++++++++++++++* |
| CNT | 48 | 1 | 1 | 13.028 | 13.099 | .072 | OS−> \|\|\|\|\|\|\|\|\|\|\|\|\|\|\|\|\|\|\|\|\|\|\|\| |
| TP | 49 | 1 | 1 | 13.028 | 13.099 | .072 | *−++++++++++++++++++++++++* |
| CPT | 50 | 1 | 1 | 13.053 | 13.124 | .072 | OS−> \|\|\|\|\|\|\|\|\|\|\|\|\|\|\|\|\|\|\|\|\|\|\|\|\| |
| TP | 51 | 1 | 1 | 13.102 | 13.159 | .058 | *−+++++++++++++++++++++++++* |
| CNT | 52 | 1 | 1 | 13.105 | 13.246 | .142 | −> \|\|\|\|\|\|\|\|\|\|\|\|\|\|\|\|\|\|\|\|\|\|\|\|\|\| |
| CPT | 53 | 1 | 1 | 13.130 | 13.246 | .117 | −> \|\|\|\|\|\|\|\|\|\|\|\|\|\|\|\|\|\|\|\|\|\|\|\|\|\| |
| TN | 54 | 1 | 1 | 13.176 | 13.194 | .019 | *−++++++++++++++++++++++++++* |
| TN | 55 | 1 | 1 | 13.196 | 13.231 | .036 | *−+++++++++++++++++++++++++++** |
| TP | 56 | 1 | 1 | 13.211 | 13.246 | .036 | *−+++++++++++++++++++++++++++++ |
| CPT | 57 | 1 | 1 | 13.253 | 13.290 | .038 | −−> \|\|\|\|\|\|\|\|\|\|\|\|\|\|\|\|\|\|\|\|\|\|\|\|\|\|\|\|\| |
| TN | 58 | 1 | 1 | 13.253 | 13.290 | .038 | *−+++++++++++++++++++++++++++++ |
| TP | 59 | 1 | 1 | 13.255 | 13.285 | .031 | *−+++++++++++++++++++++++++++++ |
| CNT | 60 | 1 | 1 | 13.255 | 13.290 | .036 | −−> \|\|\|\|\|\|\|\|\|\|\|\|\|\|\|\|\|\|\|\|\|\|\|\|\|\|\|\|\| |
| CPT | 61 | 8 | 1 | 13.295 | 13.315 | .021 | −−> \|\|\|\|\|\|\|\|\|\|\|\|\|\|\|\|\|\|\|\|\|\|\|\|\|\|\|\|\| |
| TN | 62 | 8 | 1 | 13.295 | 13.317 | .023 | *−+++++++++++++++++++++++++++++ |
| CNT | 63 | 8 | 1 | 13.340 | 13.379 | .040 | −−> \|\|\|\|\|\|\|\|\|\|\|\|\|\|\|\|\|\|\|\|\|\|\|\|\|\|\|\|\| |
| CPT | 64 | 8 | 1 | 13.341 | 13.380 | .040 | −−> \|\|\|\|\|\|\|\|\|\|\|\|\|\|\|\|\|\|\|\|\|\|\|\|\|\|\|\|\| |
| GG | 65 | 1 | 1 | 13.364 | 13.744 | .381 | Group0004 \|\|\|\|\|\|\|\|\|\|\|\|\|\|\|\|\|\|\|\|\|\|\|\|\|\|\|\|\| |
| CPT | 66 | 1 | 1 | 13.387 | 13.405 | .019 | −−> \|\|\|\|\|\|\|\|\|\|\|\|\|\|\|\|\|\|\|\|\|\|\|\|\|\|\|\|\| |
| TN | 67 | 8 | 1 | 13.387 | 13.413 | .027 | *−+++++++++++++++++++++++++++++ |
| TP | 68 | 1 | 1 | 13.444 | 13.460 | .017 | *−+++++++++++++++++++++++++++++ |
| CPT | 69 | 8 | 1 | 13.566 | 13.582 | .017 | −−> \|\|\|\|\|\|\|\|\|\|\|\|\|\|\|\|\|\|\|\|\|\|\|\|\|\|\|\|\| |
| TN | 70 | 8 | 1 | 13.574 | 13.591 | .018 | *−+++++++++++++++++++++++++++++ |
| CPT | 71 | 8 | 1 | 13.585 | 13.599 | .016 | −−> \|\|\|\|\|\|\|\|\|\|\|\|\|\|\|\|\|\|\|\|\|\|\|\|\|\|\|\|\| |
| TN | 72 | 1 | 1 | 13.656 | 13.681 | .026 | *−+++++++++++++++++++++++++++++ |
| TP | 73 | 1 | 1 | 13.656 | 13.706 | .051 | *−+++++++++++++++++++++++++++++ |
| TN | 74 | 9 | 1 | 13.704 | 13.726 | .023 | *−+++++++++++++++++++++++++++++ |
| CPT | 75 | 1 | 1 | 13.709 | 13.726 | .018 | −−> \|\|\|\|\|\|\|\|\|\|\|\|\|\|\|\|\|\|\|\|\|\|\|\|\|\|\|\|\| |
| TN | 76 | 1 | 1 | 13.734 | 13.750 | .017 | *−+++++++++++++++++++++++++++++ |
| CPT | 77 | 1 | 1 | 13.734 | 13.782 | .049 | −−> \|\|\|\|\|\|\|\|\|\|\|\|\|\|\|\|\|\|\|\|\|\|\|\|\|\|\|\|\| |
| TP | 78 | 1 | 1 | 13.734 | 13.782 | .049 | *−+++++++++++++++++++++++++++++ |
| TN | 79 | 1 | 1 | 13.768 | 13.782 | .015 | *−+++++++++++++++++++++++++++++ |
| CPT | 80 | 1 | 1 | 13.815 | 13.858 | .044 | −−> \|\|\|\|\|\|\|\|\|\|\|\|\|\|\|\|\|\|\|\|\|\|\|\|\|\|\|\|\| |
| TN | 81 | 1 | 1 | 13.840 | 13.858 | .019 | *−+++++++++++++++++++++++++++++ |
| CPT | 82 | 1 | 1 | 14.422 | 14.460 | .039 | −−> \|\|\|\|\|\|\|\|\|\|\|\|\|\|\|\|\|\|\|\|\|\|\|\|\|\|\|\|\| |
| CPT | 83 | 1 | 1 | 14.941 | 14.962 | .022 | −−> \|\|\|\|\|\|\|\|\|\|\|\|\|\|\|\|\|\|\|\|\|\|\|\|\|\|\|\|\| |
| CPT | 84 | 1 | 1 | 14.966 | 14.993 | .028 | −−> \|\|\|\|\|\|\|\|\|\|\|\|\|\|\|\|\|\|\|\|\|\|\|\|\|\|\|\|\| |
| CPT | 85 | 1 | 1 | 14.997 | 15.018 | .022 | −−> \|\|\|\|\|\|\|\|\|\|\|\|\|\|\|\|\|\|\|\|\|\|\|\|\|\|\|\|\| |
| TP | 86 | 1 | 1 | 15.795 | 15.816 | .022 | *−+++++++++++++++++++++++++++++ |
| TP | 87 | 1 | 1 | 15.996 | 16.020 | .025 | *−++*\|\|\|\|\|\|\|\|\|\|\|\|\|\|\|\|\|\|\|\|\|\|\|\|\|\| |
| TP | 88 | 1 | 1 | 16.874 | 16.898 | .025 | *−++−++++++++++++++++++++++++++ |
| CNT | 89 | 1 | 1 | 20.136 | 20.151 | .016 | −−> \|\| \|\|\|\|\|\|\|\|\|\|\|\|\|\|\|\|\|\|\|\|\|\|\|\|\|\| |
| CNT | 90 | 1 | 1 | 20.157 | 20.176 | .020 | −−> \|\| \|\|\|\|\|\|\|\|\|\|\|\|\|\|\|\|\|\|\|\|\|\|\|\|\|\| |
| GG | 91 | 1 | 1 | 21.526 | 21.852 | .327 | Group0005 \|\| \|\|\|\|\|\|\|\|\|\|\|\|\|\|\|\|\|\|\|\|\|\|\|\|\|\| |
| CPT | 92 | 1 | 1 | 22.257 | 22.304 | .048 | −−> \|\| \|\|\|\|\|\|\|\|\|\|\|\|\|\|\|\|\|\|\|\|\|\|\|\|\|\| |
| CNT | 93 | 1 | 1 | 22.275 | 22.304 | .030 | −−> \|\| \|\|\|\|\|\|\|\|\|\|\|\|\|\|\|\|\|\|\|\|\|\|\|\|\|\| |
| CNT | 94 | 1 | 1 | 22.307 | 22.322 | .016 | −−> \|\| \|\|\|\|\|\|\|\|\|\|\|\|\|\|\|\|\|\|\|\|\|\|\|\|\|\| |
| CPT | 95 | 1 | 1 | 22.307 | 22.322 | .016 | −−> \|\| \|\|\|\|\|\|\|\|\|\|\|\|\|\|\|\|\|\|\|\|\|\|\|\|\|\| |
| CNT | 96 | 1 | 1 | 22.324 | 22.338 | .015 | −−> \|\| \|\|\|\|\|\|\|\|\|\|\|\|\|\|\|\|\|\|\|\|\|\|\|\|\|\| |
| CNT | 97 | 1 | 1 | 22.351 | 22.367 | .017 | −−> \|\| \|\|\|\|\|\|\|\|\|\|\|\|\|\|\|\|\|\|\|\|\|\|\|\|\|\| |
| CPT | 98 | 8 | 1 | 22.351 | 22.367 | .017 | −−> \|\| \|\|\|\|\|\|\|\|\|\|\|\|\|\|\|\|\|\|\|\|\|\|\|\|\|\| |
| CNT | 99 | 1 | 1 | 22.376 | 22.392 | .017 | −−> \|\| \|\|\|\|\|\|\|\|\|\|\|\|\|\|\|\|\|\|\|\|\|\|\|\|\|\| |
| CPT | 100 | 8 | 1 | 22.376 | 22.392 | .017 | −−> \|\| \|\|\|\|\|\|\|\|\|\|\|\|\|\|\|\|\|\|\|\|\|\|\|\|\|\| |
| TN | 101 | 1 | 1 | 23.300 | 23.324 | .025 | *−++*\|\|\|\|\|\|\|\|\|\|\|\|\|\|\|\|\|\|\|\|\|\|\|\|\|\| |
| CPT | 102 | 8 | 1 | 23.683 | 23.716 | .034 | −−> \|\|\|\|\|\|\|\|\|\|\|\|\|\|\|\|\|\|\|\|\|\|\|\|\|\|\|\|\| |
| GG | 103 | 1 | 1 | 24.001 | 27.969 | 3.969 | Group0006 \|\|\|\|\|\|\|\|\|\|\|\|\|\|\|\|\|\|\|\|\|\|\|\|\|\|\|\|\| |
| TN | 104 | 1 | 1 | 24.119 | 24.431 | .313 | *−++++**\|\|\|\|\|\|\|\|\|\|\|\|\|\|\|\|\|\|\|\|\|\|\| |
| CPT | 105 | 1 | 1 | 24.283 | 24.352 | .070 | OS−> \|\|\|\| \|\|\|\|\|\|\|\|\|\|\|\|\|\|\|\|\|\|\|\|\|\|\| |
| TP | 106 | 1 | 1 | 24.287 | 24.431 | .145 | *−++++−−+\|\|\|\|\|\|\|\|\|\|\|\|\|\|\|\|\|\|\|\|\| |
| TN | 107 | 1 | 1 | 24.433 | 24.514 | .082 | *−++++−−*−*\|\|\|\|\|\|\|\|\|\|\|\|\|\|\|\|\|\|\|\| |
| TN | 108 | 1 | 1 | 24.516 | 24.603 | .088 | *−++++−−−−−*\|\|\|\|\|\|\|\|\|\|\|\|\|\|\|\|\|\|\| |
| TN | 109 | 8 | 1 | 24.605 | 24.703 | .099 | *−++++−−−−−−**\|\|\|\|\|\|\|\|\|\|\|\|\|\|\|\|\| |
| TN | 110 | 1 | 1 | 24.743 | 24.854 | .112 | *−++++−−−−−−−−*\|\|\|\|\|\|\|\|\|\|\|\|\|\|\|\| |
| TP | 111 | 1 | 1 | 24.863 | 25.001 | .039 | *−++++−−−−−−−−−+*++*+*\|\|\|\|\|\|\|\|\| |

The “Type” descriptor of the Ti (Id number 39) is “TP” showing that is T1 target is on the positive strand of the double-stranded genomic DNA. Because the T1 and the T2 targets have to be on the same strand, the T2 target (Id number 111) also has the type descriptor “TP”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is changed to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Group0004 (Id number 65) has the type descriptor “GG”. The same is true for Group0006 (Id number 103).

Group0004 through Group0006 are described in table 2 as

| | | Chro- | | | | |
|---|---|---|---|---|---|---|
| | COG_{—} | mo- | | | | |
| Gene Name | Id | some | Direction | Start | Stop | Length |
| YAL064C-A | — | 1 | negative | 13.364 | 13.744 | .381 |
| Group0005 | | | | | | |
| YAL064W | — | 1 | positive | 21.526 | 21.852 | .327 |
| Group0006 | | | | | | |
| FL09 | — | 1 | negative | 24.001 | 27.969 | 3.969 |

All of the data for the transient connectron 13 are pulled together in the following table that is the “terse” description of the connectron.

| Connectron Relationships - | Global_Id | Type |
|---|---|---|
| | 13 | transient |

| | | Chro- | | | | |
|---|---|---|---|---|---|---|
| Control | | mo- | C1/ | | | |
| Sequences - | Direction | some | C2_Id | Start | Stop | Length |
| | positive | 1 | 36 | 12.572 | 12.788 | .217 |

| Trigger Gene - | Name | COG_Id | Start | Stop | Length |
|---|---|---|---|---|---|
| | YAL064W-B | — | 12.047 | 12.427 | .381 |

| | | Chro- | | | | |
|---|---|---|---|---|---|---|
| Target | | mo- | | | | |
| Sequences - | Direction | some | | Start | Stop | Length |
| | | | T1_Id | | | |
| | positive | 1 | 39 | 12.572 | 12.807 | .236 |
| | | | T2_Id | | | |
| | | | 111 | 24.863 | 25.001 | .020 |

Controlled Genes

| Local_Id | Chromosome | Group | Name | COG_Id | Direction | Start | Stop | Length |
|---|---|---|---|---|---|---|---|---|
| 1 | 1 | Group0004 | YAL064C-A | — | positive | 13.364 | 13.744 | .381 |
| 2 | 1 | Group0005 | YAL064W | — | positive | 21.526 | 21.852 | .327 |
| 3 | 1 | Group0006 | FL09 | — | positive | 24.001 | 27.969 | 3.969 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 41 | negative | 12.964 | 12.908 | .057 |
| 2 | 1 | 42 | positive | 12.908 | 12.964 | .057 |
| 3 | 1 | 44 | negative | 12.986 | 12.966 | .021 |
| 4 | 1 | 45 | positive | 12.966 | 12.986 | .021 |
| 5 | 1 | 48 | negative | 13.099 | 13.028 | .072 |
| 6 | 1 | 50 | positive | 13.053 | 13.124 | .072 |
| 7 | 1 | 52 | negative | 13.246 | 13.105 | .142 |
| 8 | 1 | 53 | positive | 13.130 | 13.246 | .117 |
| 9 | 1 | 57 | positive | 13.253 | 13.290 | .038 |
| 10 | 1 | 60 | negative | 13.290 | 13.255 | .036 |
| 11 | 1 | 61 | positive | 13.295 | 13.315 | .021 |
| 12 | 1 | 63 | negative | 13.379 | 13.340 | .040 |
| 13 | 1 | 64 | positive | 13.341 | 13.380 | .040 |
| 14 | 1 | 66 | positive | 13.387 | 13.405 | .019 |
| 15 | 1 | 69 | positive | 13.566 | 13.582 | .017 |
| 16 | 1 | 71 | positive | 13.585 | 13.599 | .016 |
| 17 | 1 | 75 | positive | 13.709 | 13.726 | .018 |
| 18 | 1 | 77 | positive | 13.734 | 13.782 | .049 |
| 19 | 1 | 80 | positive | 13.815 | 13.858 | .044 |
| 20 | 1 | 82 | positive | 14.422 | 14.460 | .039 |
| 21 | 1 | 83 | positive | 14.941 | 14.962 | .022 |
| 22 | 1 | 84 | positive | 14.966 | 14.993 | .028 |
| 23 | 1 | 85 | positive | 14.997 | 15.018 | .022 |
| 24 | 1 | 89 | negative | 20.151 | 20.136 | .016 |
| 25 | 1 | 90 | negative | 20.176 | 20.157 | .020 |
| 26 | 1 | 92 | positive | 22.257 | 22.304 | .048 |
| 27 | 1 | 93 | negative | 22.304 | 22.275 | .030 |
| 28 | 1 | 94 | negative | 22.322 | 22.307 | .016 |
| 29 | 1 | 95 | positive | 22.307 | 22.322 | .016 |
| 30 | 1 | 96 | negative | 22.338 | 22.324 | .015 |
| 31 | 1 | 97 | negative | 22.367 | 22.351 | .017 |
| 32 | 1 | 98 | positive | 22.351 | 22.367 | .017 |
| 33 | 1 | 99 | negative | 22.392 | 22.376 | .017 |
| 34 | 1 | 100 | positive | 22.376 | 22.392 | .017 |
| 35 | 1 | 102 | positive | 23.683 | 23.716 | .034 |
| 36 | 1 | 105 | positive | 24.283 | 24.352 | .070 |

The verbose description of the transient connectron 13 is:

In the Saccharomyces cerevisiae complete genome the transient connectron number 13 is generated by the control sequence (C1/C2) whose identifier number is 36. This control sequence is on the positive strand of the genomic DNA of chromosome 1. The genomic start and stop positions of this control sequence are 12.572 KB and 12.788 KB with a length of 0.217 KB. Expression of the RNA for this connectron is triggered by the promotion of the gene whose name is YAL064W-B. The genomic start and stop positions of this gene are 12.047 KB and 12.427 KB and with a length of 0.381 KB. This connectron causes stabilization of a loop of DNA. The target sequences (T1-T2) are on the positive strand of the genomic DNA on chromosome 1. The identifier number of the initiating target sequence (T1) is 39. The genomic start and stop positions of this initiating target sequence are 12.572 KB and 12.807 KB with a length of 0.236 KB. The identifier number of the terminating target sequence (T2) is 111. The genomic start and stop positions of this terminating target sequence are 24.863 KB and 25.001 KB with a length of 0.020 KB.

This connectron controls the modulation of the expression of the following gene(s): (1) The gene that has the name YAL064C-A. The genomic start and stop positions of this gene are 13.364 KB and 13.744 KB with a length of 0.381 KB. (2) The gene that has the name YAL064W. The genomic start and stop positions of this gene are 21.526 KB and 21.852 KB with a length of 0.327 KB. (3) The gene that has the name FLO9. The genomic start and stop positions of this gene are 24.001 KB and 27.969 KB with a length of 3.969 KB.

This connectron controls the turning off of the expression of the following connectron(s): (1) The connectron that has the identifier 41 is on chromosome 1 in the negative direction. The genomic start and stop positions of this connectron are 12.964 KB and 12.908 KB with a length of 0.057 KB. (2) The connectron that has the identifier 42 is on chromosome 1 in the positive direction. The genomic start and stop positions of this connectron are 12.908 KB and 12.964 KB with a length of 0.057 KB. (3) The connectron that has the identifier 44 is on chromosome 1 in the negative direction. The genomic start and stop positions of this connectron are 12.986 KB and 12.966 KB with a length of 0.021 KB. (4) The connectron that has the identifier 45 is on chromosome 1 in the positive direction. The genomic start and stop positions of this connectron are 12.966 KB and 12.986 KB with a length of 0.021 KB. (5) The connectron that has the identifier 48 is on chromosome 1 in the negative direction. The genomic start and stop positions of this connectron are 13.099 KB and 13.028 KB with a length of 0.072 KB. (6) The connectron that has the identifier 50 is on chromosome 1 in the positive direction. The genomic start and stop positions of this connectron are 13.053 KB and 13.124 KB with a length of 0.072 KB. (7) The connectron that has the identifier 52 is on chromosome 1 in the negative direction. The genomic start and stop positions of this connectron are 13.246 KB and 13.105 KB with a length of 0.142 KB. (8) The connectron that has the identifier 53 is on chromosome 1 in the positive direction. The genomic start and stop positions of this connectron are 13.130 KB and 13.246 KB with a length of 0.117 KB. (9) The connectron that has the identifier 57 is on chromosome 1 in the positive direction. The genomic start and stop positions of this connectron are 13.253 KB and 13.290 KB with a length of 0.038 KB. (10) The connectron that has the identifier 60 is on chromosome 1 in the negative direction. The genomic start and stop positions of this connectron are 13.290 KB and 13.255 KB with a length of 0.036 KB. (11) The connectron that has the identifier 61 is on chromosome 1 in the positive direction. The genomic start and stop positions of this connectron are 13.295 KB and 13.315 KB with a length of 0.021 KB. (12) The connectron that has the identifier 63 is on chromosome 1 in the negative direction. The genomic start and stop positions of this connectron are 13.379 KB and 13.340 KB with a length of 0.040 KB. (13) The connectron that has the identifier 64 is on chromosome 1 in the positive direction. The genomic start and stop positions of this connectron are 13.341 KB and 13.380 KB with a length of 0.040 KB. (14) The connectron that has the identifier 66 is on chromosome 1 in the positive direction. The genomic start and stop positions of this connectron are 13.387 KB and 13.405 KB with a length of 0.019 KB. (15) The connectron that has the identifier 69 is on chromosome 1 in the positive direction. The genomic start and stop positions of this connectron are 13.566 KB and 13.582 KB with a length of 0.017 KB. (16) The connectron that has the identifier 71 is on chromosome 1 in the positive direction. The genomic start and stop positions of this connectron are 13.585 KB and 13.599 KB with a length of 0.016 KB. (17) The connectron that has the identifier 75 is on chromosome 1 in the positive direction. The genomic start and stop positions of this connectron are 13.709 KB and 13.726 KB with a length of 0.018 KB. (18) The connectron that has the identifier 77 is on chromosome 1 in the positive direction. The genomic start and stop positions of this connectron are 13.734 KB and 13.782 KB with a length of 0.049 KB. (19) The connectron that has the identifier 80 is on chromosome 1 in the positive direction. The genomic start and stop positions of this connectron are 13.815 KB and 13.858 KB with a length of 0.044 KB. (20) The connectron that has the identifier 82 is on chromosome 1 in the positive direction. The genomic start and stop positions of this connectron are 14.422 KB and 14.460 KB with a length of 0.039 KB. (21) The connectron that has the identifier 83 is on chromosome 1 in the positive direction. The genomic start and stop positions of this connectron are 14.941 KB and 14.962 KB with a length of 0.022 KB. (22) The connectron that has the identifier 84 is on chromosome 1 in the positive direction. The genomic start and stop positions of this connectron are 14.966 KB and 14.993 KB with a length of 0.028 KB. (23) The connectron that has the identifier 85 is on chromosome 1 in the positive direction. The genomic start and stop positions of this connectron are 14.997 KB and 15.018 KB with a length of 0.022 KB. (24) The connectron that has the identifier 89 is on chromosome 1 in the negative direction. The genomic start and stop positions of this connectron are 20.151 KB and 20.136 KB with a length of 0.016 KB. (25) The connectron that has the identifier 90 is on chromosome 1 in the negative direction. The genomic start and stop positions of this connectron are 20.176 KB and 20.157 KB with a length of 0.020 KB. (26) The connectron that has the identifier 92 is on chromosome 1 in the positive direction. The genomic start and stop positions of this connectron are 22.257 KB and 22.304 KB with a length of 0.048 KB. (27) The connectron that has the identifier 93 is on chromosome 1 in the negative direction. The genomic start and stop positions of this connectron are 22.304 KB and 22.275 KB with a length of 0.030 KB. (28) The connectron that has the identifier 94 is on chromosome 1 in the negative direction. The genomic start and stop positions of this connectron are 22.322 KB and 22.307 KB with a length of 0.016 KB. (29) The connectron that has the identifier 95 is on chromosome 1 in the positive direction. The genomic start and stop positions of this connectron are 22.307 KB and 22.322 KB with a length of 0.016 KB. (30) The connectron that has the identifier 96 is on chromosome 1 in the negative direction. The genomic start and stop positions of this connectron are 22.338 KB and 22.324 KB with a length of 0.015 KB. (31) The connectron that has the identifier 97 is on chromosome 1 in the negative direction. The genomic start and stop positions of this connectron are 22.367 KB and 22.351 KB with a length of 0.017 KB. (32) The connectron that has the identifier 98 is on chromosome 1 in the positive direction. The genomic start and stop positions of this connectron are 22.351 KB and 22.367 KB with a length of 0.017 KB. (33) The connectron that has the identifier 99 is on chromosome 1 in the negative direction. The genomic start and stop positions of this connectron are 22.392 KB and 22.376 KB with a length of 0.017 KB. (34) The connectron that has the identifier 100 is on chromosome 1 in the positive direction. The genomic start and stop positions of this connectron are 22.376 KB and 22.392 KB with a length of 0.017 KB. (35) The connectron that has the identifier 102 is on chromosome 1 in the positive direction. The genomic start and stop positions of this connectron are 23.683 KB and 23.716 KB with a length of 0.034 KB. (36) The connectron that has the identifier 105 is on chromosome 1 in the positive direction. The genomic start and stop positions of this connectron are 24.283 KB and 24.352 KB with a length of 0.070 KB.

### Addition of a Synthetic Connectron of Type 2 from Table 2

A synthetic connectron of type 2 in which the C1 and C2 are synthetic elements, the T1 is a synthetic element and the T2 is a natural element can be introduced into the E. coli genome by modifying the DNA double-strand sequences at 4626.130 kb through 4626.166 kb for a length of 0.036 kb and by modifying the DNA double-strand sequence at 705.150 kb through 705.203 kb for a length of 0.054 kb.

Connectron 62521 is an example of a synthetic transient connectron. It is described as

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 62521 | 1 | 4651a | 4651a | 1 | 811a | 975 | transient |

The C1/C2 source of the transient connectron 62521 is represented in Table 1 as

| Type | Num | Jobno | Chr | Start | Stop | Length | GeneName |
|---|---|---|---|---|---|---|---|
| CPT | 4651a | 1 | 1 | 4626.130 | 4626.166 | .037 | \|\|\| \|\| \| \|\|\|\| \|\| \| \|\| |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB).

The T1-T2 target of the transient connectron 62521 as shown in Table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TN | 811a | 1 | 1 | 705.150 | 705.203 | .054 | *−* |
| TP | 812 | 1 | 1 | 705.122 | 705.141 | .020 | *−+ |
| GG | 813 | 1 | 1 | 705.316 | 706.980 | 1.665 | Group0150 \| |
| TN | 814 | 1 | 1 | 707.013 | 707.028 | .016 | *−+ |
| TP | 815 | 1 | 1 | 707.014 | 707.028 | .015 | *−+ |
| CPT | 816 | 1 | 1 | 707.055 | 707.109 | .055 | −−> \| |
| TN | 817 | 1 | 1 | 707.055 | 707.109 | .055 | *−+ |
| TP | 818 | 1 | 1 | 707.055 | 707.109 | .055 | *−+ |
| CPT | 819 | 1 | 1 | 707.112 | 707.162 | .051 | OS−> \| |
| TN | 820 | 1 | 1 | 707.112 | 707.162 | .051 | *−+ |
| TP | 821 | 1 | 1 | 707.112 | 707.162 | .051 | *−+ |
| GG | 822 | 1 | 1 | 707.557 | 710.688 | 3.131 | Group0151 \| |
| CNT | 823 | 1 | 1 | 710.633 | 710.647 | .015 | OS−> \| |
| TN | 824 | 1 | 1 | 710.633 | 710.647 | .015 | *−+ |
| CNT | 825 | 1 | 1 | 710.652 | 710.666 | .015 | −−> \| |
| GG | 826 | 1 | 1 | 710.828 | 712.755 | 1.927 | Group0152 \| |
| TN | 827 | 1 | 1 | 712.281 | 712.296 | .016 | *−+ |
| GG | 828 | 1 | 1 | 712.781 | 714.421 | 1.641 | Group0153 \| |
| TN | 829 | 1 | 1 | 714.481 | 714.518 | .038 | *−+ |
| CPT | 830 | 1 | 1 | 714.481 | 714.519 | .039 | OS−> \| |
| TP | 831 | 1 | 1 | 714.482 | 714.519 | .038 | *−+ |
| CPT | 832 | 1 | 1 | 714.524 | 714.540 | .017 | −−> \| |
| TN | 833 | 1 | 1 | 714.524 | 714.540 | .017 | *−+ |
| TP | 834 | 1 | 1 | 714.524 | 714.540 | .017 | *−+ |
| CNT | 835 | 1 | 1 | 714.543 | 714.593 | .051 | OS−> \| |
| TN | 836 | 1 | 1 | 714.543 | 714.593 | .051 | *−+ |
| TP | 837 | 1 | 1 | 714.543 | 714.593 | .051 | *−+ |
| CPT | 838 | 1 | 1 | 714.544 | 714.606 | .063 | OS−> \| |
| GG | 839 | 1 | 1 | 714.635 | 715.928 | 1.293 | Group0154 \| |
| GN | 840 | 1 | 1 | 715.170 | 715.532 | .363 | ybfg \| |
| TN | 841 | 1 | 1 | 716.085 | 716.103 | .019 | *−+ |
| TP | 842 | 1 | 1 | 716.085 | 716.103 | .019 | *−+ |
| GG | 843 | 1 | 1 | 716.169 | 724.202 | 8.033 | Group0155 \| |
| CNT | 844 | 1 | 1 | 720.278 | 720.296 | .019 | −−> \| |
| GN | 845 | 1 | 1 | 720.279 | 724.202 | 3.924 | kdpe \| |
| TP | 846 | 1 | 1 | 721.769 | 721.783 | .015 | *−+ |
| TN | 847 | 1 | 1 | 721.812 | 721.827 | .016 | *−+ |
| GG | 848 | 1 | 1 | 724.211 | 727.955 | 3.744 | Group0156 \| |
| TP | 849 | 1 | 1 | 726.665 | 726.679 | .015 | *−+ |
| GG | 850 | 1 | 1 | 728.357 | 733.325 | 4.968 | Group0157 \| |
| GP | 851 | 1 | 1 | 728.806 | 733.325 | 4.520 | rhsc \| |
| CPT | 852 | 1 | 1 | 729.408 | 729.446 | .039 | −−> \| |
| TP | 853 | 1 | 1 | 730.270 | 730.284 | .015 | *−+ |
| CPT | 854 | 1 | 1 | 730.443 | 730.457 | .015 | −−> \| |
| CPT | 855 | 1 | 1 | 730.468 | 730.482 | .015 | −−> \| |
| CPT | 856 | 1 | 1 | 730.484 | 730.507 | .024 | −−> \| |
| CPT | 857 | 1 | 1 | 731.251 | 731.290 | .040 | −−> \| |
| TP | 858 | 1 | 1 | 731.475 | 731.508 | .034 | *−+ |
| TP | 859 | 1 | 1 | 731.512 | 731.562 | .051 | *−+ |
| TP | 860 | 1 | 1 | 731.564 | 731.693 | .130 | *−+ |
| TP | 861 | 1 | 1 | 731.698 | 731.755 | .058 | *−+ |
| TP | 862 | 1 | 1 | 731.757 | 731.819 | .063 | *−+ |
| TP | 863 | 1 | 1 | 731.821 | 731.903 | .083 | *−+ |
| TP | 864 | 1 | 1 | 731.906 | 732.033 | .128 | *−+ |
| CPT | 865 | 1 | 1 | 731.994 | 732.016 | .023 | −−> \| |
| CPT | 866 | 1 | 1 | 732.019 | 732.034 | .016 | −−> \| |
| TP | 867 | 1 | 1 | 732.035 | 732.050 | .016 | *−+ |
| TP | 868 | 1 | 1 | 732.052 | 732.179 | .128 | *−+ |
| CPT | 869 | 1 | 1 | 732.065 | 732.179 | .115 | OS−> \| |
| CPT | 870 | 1 | 1 | 732.198 | 732.212 | .015 | −−> \| |
| TP | 871 | 1 | 1 | 732.198 | 732.212 | .015 | *−\| |
| CPT | 872 | 1 | 1 | 732.214 | 732.233 | .020 | −−> \| |
| TP | 873 | 1 | 1 | 732.214 | 732.233 | .020 | *−+ |
| CPT | 874 | 1 | 1 | 732.235 | 732.283 | .049 | −−> \| |
| TP | 875 | 1 | 1 | 732.235 | 732.283 | .049 | *−+ |
| CPT | 876 | 1 | 1 | 732.306 | 732.326 | .021 | −−> \| |
| TP | 877 | 1 | 1 | 732.306 | 732.326 | .021 | *−+ |
| CPT | 878 | 1 | 1 | 732.328 | 732.452 | .125 | −−> \| |
| TP | 879 | 1 | 1 | 732.328 | 732.482 | .155 | *−+ |
| CPT | 880 | 1 | 1 | 732.454 | 732.482 | .029 | −−> \| |
| CPT | 881 | 1 | 1 | 732.488 | 732.519 | .032 | −−> \| |
| TN | 882 | 1 | 1 | 733.330 | 733.344 | .015 | *−+ |
| TP | 883 | 1 | 1 | 733.330 | 733.344 | .015 | *−+ |
| TP | 884 | 1 | 1 | 733.371 | 733.513 | .143 | *−+ |
| GG | 885 | 1 | 1 | 733.443 | 735.442 | 2.000 | Group0158 \| |
| TP | 886 | 1 | 1 | 733.515 | 733.564 | .050 | *−+ |
| TP | 887 | 1 | 1 | 733.569 | 733.626 | .058 | *−+ |
| TP | 888 | 1 | 1 | 733.628 | 733.645 | .018 | *−+ |
| TP | 889 | 1 | 1 | 733.647 | 733.696 | .050 | *−+ |
| TP | 890 | 1 | 1 | 733.698 | 733.774 | .077 | *−+ |
| TP | 891 | 1 | 1 | 733.776 | 733.904 | .129 | *−+ |
| CPT | 892 | 1 | 1 | 733.865 | 733.887 | .023 | OS−> \| |
| CPT | 893 | 1 | 1 | 733.890 | 733.905 | .016 | −−> \| |
| TP | 894 | 1 | 1 | 733.906 | 734.050 | .145 | *−+ |
| CPT | 895 | 1 | 1 | 733.936 | 734.050 | .115 | −−> \| |
| TP | 896 | 1 | 1 | 734.052 | 734.075 | .024 | *−+ |
| CPT | 897 | 1 | 1 | 734.052 | 734.139 | .088 | −−> \| |
| TP | 898 | 1 | 1 | 734.084 | 734.139 | .056 | *−+ |
| TP | 899 | 1 | 1 | 734.149 | 734.179 | .031 | *−+ |
| CPT | 900 | 1 | 1 | 734.164 | 734.321 | .158 | −−> \| |
| TP | 901 | 1 | 1 | 734.192 | 734.321 | .130 | *−+ |
| TP | 902 | 1 | 1 | 734.323 | 734.378 | .056 | *−+ |
| CPT | 903 | 1 | 1 | 734.323 | 734.381 | .059 | −−> \| |
| CPT | 904 | 1 | 1 | 735.525 | 735.569 | .045 | −−> \| |
| TP | 905 | 1 | 1 | 735.525 | 735.569 | .045 | *−+ |
| CPT | 906 | 1 | 1 | 735.606 | 735.703 | .098 | −−> \| |
| TP | 907 | 1 | 1 | 735.606 | 735.703 | .098 | *−+ |
| TP | 908 | 1 | 1 | 735.707 | 735.721 | .015 | *−+ |
| CPT | 909 | 1 | 1 | 735.707 | 735.728 | .022 | −−> \| |
| TP | 910 | 1 | 1 | 735.749 | 735.803 | .055 | *−+ |
| CPT | 911 | 1 | 1 | 735.807 | 735.964 | .158 | OS−> \| |
| TP | 912 | 1 | 1 | 735.814 | 735.895 | .082 | *−+ |
| TP | 913 | 1 | 1 | 735.905 | 735.934 | .030 | *−+ |
| TP | 914 | 1 | 1 | 735.938 | 736.083 | .146 | *−+ |
| CPT | 915 | 1 | 1 | 735.986 | 736.083 | .098 | OS−> \| |
| TP | 916 | 1 | 1 | 736.152 | 736.173 | .022 | *−+ |
| TP | 917 | 1 | 1 | 736.187 | 736.217 | .031 | *−+ |
| TP | 918 | 1 | 1 | 736.219 | 736.265 | .047 | *−+ |
| TP | 919 | 1 | 1 | 736.288 | 736.334 | .047 | *−+ |
| GG | 920 | 1 | 1 | 736.327 | 737.184 | .858 | Group0159 \| |
| TP | 921 | 1 | 1 | 736.349 | 736.469 | .121 | *−+ |
| TP | 922 | 1 | 1 | 736.516 | 736.628 | .113 | *−+ |
| TP | 923 | 1 | 1 | 736.630 | 736.645 | .016 | *−+ |
| TP | 924 | 1 | 1 | 736.654 | 736.792 | .139 | *−+ |
| TN | 925 | 1 | 1 | 736.654 | 736.809 | .156 | *−+ |
| TN | 926 | 1 | 1 | 736.816 | 736.832 | .017 | *−+ |
| CPT | 927 | 1 | 1 | 737.017 | 737.032 | .016 | −−> \| |
| CPT | 928 | 1 | 1 | 737.035 | 737.057 | .023 | −−> \| |
| TN | 929 | 1 | 1 | 737.168 | 737.188 | .021 | *−++ |
| TP | 930 | 1 | 1 | 737.196 | 737.290 | .095 | *−+ |
| CPT | 931 | 1 | 1 | 737.261 | 737.275 | .015 | −−> \| |
| CPT | 932 | 1 | 1 | 737.278 | 737.292 | .015 | −−> \| |
| TP | 933 | 1 | 1 | 737.292 | 737.393 | .102 | *−+ |
| GG | 934 | 1 | 1 | 737.315 | 738.076 | .762 | Group0160 \| |
| TP | 935 | 1 | 1 | 737.411 | 737.475 | .065 | *−+ |
| TP | 936 | 1 | 1 | 737.479 | 737.557 | .079 | *−+ |
| TP | 937 | 1 | 1 | 737.580 | 737.597 | .018 | *−+ |
| TP | 938 | 1 | 1 | 737.605 | 737.626 | .022 | *−+ |
| TP | 939 | 1 | 1 | 737.633 | 737.648 | .016 | *−+ |
| TP | 940 | 1 | 1 | 737.653 | 737.679 | .027 | *−+ |
| TP | 941 | 1 | 1 | 737.683 | 737.752 | .070 | *−+ |
| TP | 942 | 1 | 1 | 737.798 | 737.822 | .025 | *−+ |
| TP | 943 | 1 | 1 | 737.828 | 737.895 | .068 | *−+ |
| TP | 944 | 1 | 1 | 737.897 | 737.931 | .035 | *−+ |
| CPT | 945 | 1 | 1 | 737.930 | 737.967 | .038 | −−> \| |
| TP | 946 | 1 | 1 | 737.936 | 737.967 | .032 | *−+ |
| TP | 947 | 1 | 1 | 737.970 | 737.988 | .019 | *−+ |
| TN | 948 | 1 | 1 | 737.990 | 738.015 | .026 | *−+ |
| TP | 949 | 1 | 1 | 737.990 | 738.015 | .026 | *−+ |
| TN | 950 | 1 | 1 | 738.019 | 738.051 | .033 | *−+ |
| TP | 951 | 1 | 1 | 738.019 | 738.051 | .033 | *−++ |
| TP | 952 | 1 | 1 | 738.053 | 738.074 | .022 | *−+ |
| TN | 953 | 1 | 1 | 738.058 | 738.074 | .017 | *−+ |
| TN | 954 | 1 | 1 | 738.107 | 738.126 | .020 | *−+ |
| GG | 955 | 1 | 1 | 738.224 | 740.148 | 1.925 | Group0161 \| |
| TN | 956 | 1 | 1 | 740.172 | 740.213 | .042 | *−+ |
| CNT | 957 | 1 | 1 | 740.172 | 740.290 | .119 | OS−> \| |
| CPT | 958 | 1 | 1 | 740.172 | 740.290 | .119 | OS−> \| |
| TP | 959 | 1 | 1 | 740.173 | 740.213 | .041 | *−+ |
| TP | 960 | 1 | 1 | 740.216 | 740.241 | .026 | *−+ |
| TN | 961 | 1 | 1 | 740.216 | 740.288 | .073 | *−+ |
| TP | 962 | 1 | 1 | 740.244 | 740.288 | .045 | *−+ |
| GG | 963 | 1 | 1 | 740.298 | 757.628 | 17.330 | Group0162 \| |
| GP | 964 | 1 | 1 | 742.816 | 745.122 | 2.307 | ybgj \| |
| CPT | 965 | 1 | 1 | 744.643 | 744.657 | .015 | −−> \| |
| CPT | 966 | 1 | 1 | 744.664 | 744.678 | .015 | −−> \| |
| GP | 967 | 1 | 1 | 754.400 | 756.896 | 2.497 | sdhc \| |
| CPT | 968 | 1 | 1 | 755.680 | 755.714 | .035 | −−> \| |
| GP | 969 | 1 | 1 | 756.912 | 757.628 | .717 | sdhb \| |
| TN | 970 | 1 | 1 | 757.626 | 757.712 | .087 | *−+ |
| CPT | 971 | 1 | 1 | 757.628 | 757.712 | .085 | OS−> \| |
| TP | 972 | 1 | 1 | 757.629 | 757.712 | .084 | *−+ |
| GG | 973 | 1 | 1 | 757.687 | 760.730 | 3.044 | Group0163 \| |
| CPT | 974 | 1 | 1 | 757.717 | 757.736 | .020 | −−> \| |
| TN | 975 | 1 | 1 | 757.718 | 757.753 | .036 | *−+ |

The “Type” descriptor of the T1 (Id number 811a) is “TN” showing that is T1 target is on the negative strand of the double-stranded genomic DNA. Because the T1 and the T2 targets have to be on the same strand, the T2 target (Id number 975) also has the type descriptor “TN”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is chanted to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Group0150 through Group0163 are described in Table 2 as

| Gene_Name | COG_Id | | Chromosome | Direction | Start | Stop | Length |
|---|---|---|---|---|---|---|---|
| Group0150 | | | | | | | |
| glns | COG0008 | b | 1 | positive | 705.316 | 706.980 | 1.665 |
| Group0151 | | | | | | | |
| ybfm | — | b | 1 | positive | 707.557 | 708.963 | 1.407 |
| ybfn | — | b | 1 | positive | 709.013 | 709.339 | .327 |
| fur | COG0735 | b | 1 | negative | 709.423 | 709.869 | .447 |
| flda | COG0716 | b | 1 | negative | 710.158 | 710.688 | .531 |
| Group0152 | | | | | | | |
| ybfe | — | b | 1 | negative | 710.828 | 711.190 | .363 |
| ybff | COG0596 | b | 1 | negative | 711.261 | 712.025 | .765 |
| seqa | COG3057 | b | 1 | positive | 712.210 | 712.755 | .546 |
| Group0153 | | | | | | | |
| pgm | COG0033 | b | 1 | positive | 712.781 | 714.421 | 1.641 |
| Group0154 | | | | | | | |
| ybfp | — | b | 1 | positive | 714.635 | 715.129 | .495 |
| ybfg | — | b | 1 | negative | 715.170 | 715.532 | .363 |
| ybfh | — | b | 1 | negative | 715.611 | 715.928 | .318 |
| Group0155 | | | | | | | |
| pote | COG0531 | b | 1 | negative | 716.169 | 719.683 | 3.515 |
| kdpe | COG0745 | b | 1 | negative | 720.279 | 724.202 | 3.924 |
| Group0156 | | | | | | | |
| kdpb | COG2216 | b | 1 | negative | 724.211 | 726.259 | 2.049 |
| kdpa | COG2060 | b | 1 | negative | 726.282 | 727.955 | 1.674 |
| Group0157 | | | | | | | |
| ybfa | — | b | 1 | positive | 728.357 | 728.563 | .207 |
| rhsc | — | b | 1 | positive | 728.806 | 733.325 | 4.520 |
| Group0158 | | | | | | | |
| b0703 | COG3209 | b | 1 | positive | 733.443 | 735.442 | 2.000 |
| Group0159 | | | | | | | |
| ybfl | — | b | 1 | positive | 736.327 | 737.184 | .858 |
| Group0160 | | | | | | | |
| ybfd | — | b | 1 | positive | 737.315 | 738.076 | .762 |
| Group0161 | | | | | | | |
| ybga | COG3272 | b | 1 | positive | 738.224 | 740.148 | 1.925 |
| Group0162 | | | | | | | |
| ybgh | COG3104 | b | 1 | negative | 740.298 | 741.779 | 1.482 |
| ybgi | COG0327 | b | 1 | positive | 742.050 | 742.793 | .744 |
| ybgj | COG2049 | b | 1 | positive | 742.816 | 745.122 | 2.307 |
| nei | COG0266 | b | 1 | positive | 745.158 | 745.949 | .792 |
| abrb | — | b | 1 | negative | 745.946 | 747.037 | 1.092 |
| ybgo | — | b | 1 | negative | 747.144 | 748.930 | 1.787 |
| ybgq | — | b | 1 | negative | 748.945 | 751.401 | 2.457 |
| ybgd | — | b | 1 | negative | 751.452 | 752.018 | .567 |
| glta | COG0372 | b | 1 | negative | 752.408 | 753.691 | 1.284 |
| sdhc | COG2009 | b | 1 | positive | 754.400 | 756.896 | 2.497 |
| sdhb | COG0479 | b | 1 | positive | 756.912 | 757.628 | .717 |
| Group0163 | | | | | | | |
| b0725 | — | b | 1 | positive | 757.687 | 760.730 | 3.044 |

All of the data for the transient connectron 62521 are pulled together in the following table that is the “terse” description of the connectron

| Connectron Relationships - | Global_Id | Type |
|---|---|---|
| | 62521 | transient |

| | | Chro- | | | | |
|---|---|---|---|---|---|---|
| Control | Direc- | mo- | C1/ | | | |
| Sequences - | tion | some | C2_Id | Start | Stop | Length |
| | positive | 1 | 4651a | 4626.130 | 4626.166 | .036 |

| Trigger | | | | | |
|---|---|---|---|---|---|
| Gene - | Name | COG_Id | Start | Stop | Length |
| | sms | | 4623.481 | 4626.116 | 2.636 |

| | | Chro- | | | | |
|---|---|---|---|---|---|---|
| Target | Direc- | mo- | | | | |
| Sequences - | tion | some | | Start | Stop | Length |
| | | | T1_Id | | | |
| | negative | 1 | 811a | 705.150 | 705.203 | .054 |
| | | | T2_Id | | | |
| | | | 975 | 757.753 | 757.718 | .036 |

Controlled Genes

| Local_Id | Chromosome | Group | Name | COG_Id | Direction | Start | Stop | Length |
|---|---|---|---|---|---|---|---|---|
| 1 | 1 | Group0150 | gins | COG0008 | positive | 705.316 | 706.980 | 1.665 |
| 2 | 1 | Group0151 | ybfm | — | positive | 707.557 | 710.688 | 3.131 |
| 3 | 1 | Group0151 | ybfn | — | positive | 709.013 | 709.339 | .327 |
| 4 | 1 | Group0151 | fur | COG0735 | negative | 709.869 | 709.423 | .447 |
| 5 | 1 | Group0151 | flda | COG0716 | negative | 710.688 | 710.158 | .531 |
| 6 | 1 | Group0152 | ybfe | — | positive | 710.828 | 712.755 | 1.927 |
| 7 | 1 | Group0152 | ybff | COG0596 | negative | 712.025 | 711.261 | .765 |
| 8 | 1 | Group0152 | seqa | COG3057 | positive | 712.210 | 712.755 | .546 |
| 9 | 1 | Group0153 | pgm | COG0033 | positive | 712.781 | 714.421 | 1.641 |
| 10 | 1 | Group0154 | ybfp | — | positive | 714.635 | 715.928 | 1.293 |
| 11 | 1 | Group0154 | ybfg | — | negative | 715.532 | 715.170 | .363 |
| 12 | 1 | Group0154 | ybfh | — | negative | 715.928 | 715.611 | .318 |
| 13 | 1 | Group0155 | pote | COG0531 | positive | 716.169 | 724.202 | 8.033 |
| 14 | 1 | Group0155 | kdpe | COG0745 | negative | 724.202 | 720.279 | 3.924 |
| 15 | 1 | Group0156 | kdpb | COG2216 | positive | 724.211 | 727.955 | 3.744 |
| 16 | 1 | Group0156 | kdpa | COG2060 | negative | 727.955 | 726.282 | 1.674 |
| 17 | 1 | Group0157 | ybfa | — | positive | 728.357 | 733.325 | 4.968 |
| 18 | 1 | Group0157 | rhsc | — | positive | 728.806 | 733.325 | 4.520 |
| 19 | 1 | Group0158 | b0703 | COG3209 | positive | 733.443 | 735.442 | 2.000 |
| 20 | 1 | Group0159 | ybfl | — | positive | 736.327 | 737.184 | .858 |
| 21 | 1 | Group0160 | ybfd | — | positive | 737.315 | 738.076 | .762 |
| 22 | 1 | Group0161 | ybga | COG3272 | positive | 738.224 | 740.148 | 1.925 |
| 23 | 1 | Group0162 | ybgh | COG3104 | positive | 740.298 | 757.628 | 17.330 |
| 24 | 1 | Group0162 | ybgi | COG0327 | positive | 742.050 | 742.793 | .744 |
| 25 | 1 | Group0162 | ybgj | COG2049 | positive | 742.816 | 745.122 | 2.307 |
| 26 | 1 | Group0162 | nei | COG0266 | positive | 745.158 | 745.949 | .792 |
| 27 | 1 | Group0162 | abrb | — | negative | 747.037 | 745.946 | 1.092 |
| 28 | 1 | Group0162 | ybgo | — | negative | 748.930 | 747.144 | 1.787 |
| 29 | 1 | Group0162 | ybgq | — | negative | 751.401 | 748.945 | 2.457 |
| 30 | 1 | Group0162 | ybgd | — | negative | 752.018 | 751.452 | .567 |
| 31 | 1 | Group0162 | glta | COG0372 | negative | 753.691 | 752.408 | 1.284 |
| 32 | 1 | Group0162 | sdhc | COG2009 | positive | 754.400 | 756.896 | 2.497 |
| 33 | 1 | Group0162 | sdhb | COG0479 | positive | 756.912 | 757.628 | .717 |
| 34 | 1 | Group0163 | b0725 | — | positive | 757.687 | 760.730 | 3.044 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 816 | positive | 707.055 | 707.109 | .055 |
| 2 | 1 | 819 | positive | 707.112 | 707.162 | .051 |
| 3 | 1 | 823 | negative | 710.647 | 710.633 | .015 |
| 4 | 1 | 825 | negative | 710.666 | 710.652 | .015 |
| 5 | 1 | 830 | positive | 714.481 | 714.519 | .039 |
| 6 | 1 | 832 | positive | 714.524 | 714.540 | .017 |
| 7 | 1 | 835 | negative | 714.593 | 714.543 | .051 |
| 8 | 1 | 838 | positive | 714.544 | 714.606 | .063 |
| 9 | 1 | 844 | negative | 720.296 | 720.278 | .019 |
| 10 | 1 | 852 | positive | 729.408 | 729.446 | .039 |
| 11 | 1 | 854 | positive | 730.443 | 730.457 | .015 |
| 12 | 1 | 855 | positive | 730.468 | 730.482 | .015 |
| 13 | 1 | 856 | positive | 730.484 | 730.507 | .024 |
| 14 | 1 | 857 | positive | 731.251 | 731.290 | .040 |
| 15 | 1 | 865 | positive | 731.994 | 732.016 | .023 |
| 16 | 1 | 866 | positive | 732.019 | 732.034 | .016 |
| 17 | 1 | 869 | positive | 732.065 | 732.179 | .115 |
| 18 | 1 | 870 | positive | 732.198 | 732.212 | .015 |
| 19 | 1 | 872 | positive | 732.214 | 732.233 | .020 |
| 20 | 1 | 874 | positive | 732.235 | 732.283 | .049 |
| 21 | 1 | 876 | positive | 732.306 | 732.326 | .021 |
| 22 | 1 | 878 | positive | 732.328 | 732.452 | .125 |
| 23 | 1 | 880 | positive | 732.454 | 732.482 | .029 |
| 24 | 1 | 881 | positive | 732.488 | 732.519 | .032 |
| 25 | 1 | 892 | positive | 733.865 | 733.887 | .023 |
| 26 | 1 | 893 | positive | 733.890 | 733.905 | .016 |
| 27 | 1 | 895 | positive | 733.936 | 734.050 | .115 |
| 28 | 1 | 897 | positive | 734.052 | 734.139 | .088 |
| 29 | 1 | 900 | positive | 734.164 | 734.321 | .158 |
| 30 | 1 | 903 | positive | 734.323 | 734.381 | .059 |
| 31 | 1 | 904 | positive | 735.525 | 735.569 | .045 |
| 32 | 1 | 906 | positive | 735.606 | 735.703 | .098 |
| 33 | 1 | 909 | positive | 735.707 | 735.728 | .022 |
| 34 | 1 | 911 | positive | 735.807 | 735.964 | .158 |
| 35 | 1 | 915 | positive | 735.986 | 736.083 | .098 |
| 36 | 1 | 927 | positive | 737.017 | 737.032 | .016 |
| 37 | 1 | 928 | positive | 737.035 | 737.057 | .023 |
| 38 | 1 | 931 | positive | 737.261 | 737.275 | .015 |
| 39 | 1 | 932 | positive | 737.278 | 737.292 | .015 |
| 40 | 1 | 945 | positive | 737.930 | 737.967 | .038 |
| 41 | 1 | 957 | negative | 740.290 | 740.172 | .119 |
| 42 | 1 | 958 | positive | 740.172 | 740.290 | .119 |
| 43 | 1 | 965 | positive | 744.643 | 744.657 | .015 |
| 44 | 1 | 966 | positive | 744.664 | 744.678 | .015 |
| 45 | 1 | 968 | positive | 755.680 | 755.714 | .035 |
| 46 | 1 | 971 | positive | 757.628 | 757.712 | .085 |
| 47 | 1 | 974 | positive | 757.717 | 757.736 | .020 |

The verbose description of the synthetic connectron 62521 is:

In the Escherichia coli K-12 MG1655 complete genome the transient connectron number 62521 is generated by the control sequence (C1/C2) whose identifier number is 4651a. This control sequence is on the positive strand of the genomic DNA of chromosome 1. The genomic start and stop positions of this control sequence are 4626.130 KB and 4626.166 KB with a length of 0.036 KB. Expression of the RNA for this connectron is triggered by the promotion of the gene whose name is sms. The genomic start and stop positions of this gene are 4623.481 KB and 4626.116 KB and with a length of 2.636 KB. This connectron causes stabilization of a loop of DNA. The target sequences (T1-T2) are on the negative strand of the genomic DNA on chromosome 1. The identifier number of the initiating target sequence (T1) is 811a. The genomic start and stop positions of this initiating target sequence are 705.150 KB and 705.203 KB with a length of 0.054 KB. The identifier number of the terminating target sequence (T2) is 975. The genomic start and stop positions of this terminating target sequence are 757.753 KB and 757.718 KB with a length of 0.036 KB.

This connectron controls the modulation of the expression of the following gene(s): (1) The gene that has the name glns and with a COG identifier of COG0008. The genomic start and stop positions of this gene are 705.316 KB and 706.980 KB with a length of 1.665 KB. (2) The gene that has the name ybfm. The genomic start and stop positions of this gene are 707.557 KB and 710.688 KB with a length of 3.131 KB. (3) The gene that has the name ybfn. The genomic start and stop positions of this gene are 709.013 KB and 709.339 KB with a length of 0.327 KB. (4) The gene that has the name fur and with a COG identifier of COG0735. The genomic start and stop positions of this gene are 709.869 KB and 709.432 KB with a length of 0.447 KB. (5) The gene that has the name flda and with a COG identifier of COG0716. The genomic start and stop positions of this gene are 710.688 KB and 710.158 KB with a length of 0.531 KB. (6) The gene that has the name ybfe. The genomic start and stop positions of this gene are 710.828 KB and 712.755 KB with a length of 1.927 KB. (7) The gene that has the name ybff and with a COG identifier of COG0596. The genomic start and stop positions of this gene are 712.025 KB and 711.261 KB with a length of 0.765 KB. (8) The gene that has the name seqa and with a COG identifier of COG3057. The genomic start and stop positions of this gene are 712.210 KB and 712.755 KB with a length of 0.546 KB. (9) The gene that has the name pgm and with a COG identifier of COG0033. The genomic start and stop positions of this gene are 712.781 KB and 714.421 KB with a length of 1.641 KB. (10) The gene that has the name ybfp. The genomic start and stop positions of this gene are 714.635 KB and 715.928 KB with a length of 1.293 KB. (11) The gene that has the name ybfg. The genomic start and stop positions of this gene are 715.532 KB and 715.170 KB with a length of 0.363 KB. (12) The gene that has the name ybfh. The genomic start and stop positions of this gene are 715.928 KB and 715.611 KB with a length of 0.318 KB. (13) The gene that has the name pote and with a COG identifier of COG0531. The genomic start and stop positions of this gene are 716.169 KB and 724.202 KB with a length of 8.033 KB. (14) The gene that has the name kdpe and with a COG identifier of COG0745. The genomic start and stop positions of this gene are 724.202 KB and 720.279 KB with a length of 3.924 KB. (15) The gene that has the name kdpb and with a COG identifier of COG2216. The genomic start and stop positions of this gene are 724.211 KB and 727.955 KB with a length of 3.744 KG. (16) The gene that has the name kdpa and with a COG identifier of COG2060. The genomic start and stop positions of this gene are 727.955 KB and 726.282 KB with a length of 1.674 KB. (17) The gene that has the name ybfa. The genomic start and stop positions of this gene are 728.357 KB and 733.325 KB with a length of 4.968 KB. (18) The gene that has the name rhsc. The genomic start and stop positions of this gene are 728.806 KOB and 733.325 KB with a length of 4.520 KB. (19) The gene that has the name b0703 and with a COG identifier of COG3209. The genomic start and stop positions of this gene are 733.443 KB and 735.442 KB with a length of 2.000 KB. (20) The gene that has the name ybfl. The genomic start and stop positions of this gene are 736.327 KB and 737.184 KB with a length of 0.858 KB. (21) The gene that has the name ybfd. The genomic start and stop positions of this gene are 737.315 KB and 738.076 KB with a length of 0.762 KB. (22) The gene that has the name ybga and with a COG identifier of COG3272. The genomic start and stop positions of this gene are 738.224 KB and 740.148 KB with a length of 1.925 KB. (23) The gene that has the name ybgh and with a COG identifier of COG3104. The genomic start and stop positions of this gene are 740.298 KB and 757.628 KB with a length of 17.330 KB. (24) The gene that has the name ybgi and with a COG identifier of COG0327. The genomic start and stop positions of this gene are 742.050 KB and 742.793 KB with a length of 0.744 KB. (25) The gene that has the name ybgj and with a COG identifier of COG2049. The genomic start and stop positions of this gene are 742.816 KB and 745.122 KB with a length of 2.307 KB. (26) The gene that has the name nei and with a COG identifier of COG0266. The genomic start and stop positions of this gene are 745.158 KB and 745.949 KB with a length of 0.792 KB. (27) The gene that has the name abrb. The genomic start and stop positions of this gene are 747.037 KB and 745.946 KB with a length of 1.092 KB. (28) The gene that has the name ybgo. The genomic start and stop positions of this gene are 748.930 KB and 747.144 KB with a length of 1.787 KB. (29) The gene that has the name ybgq. The genomic start and stop positions of this gene are 751.410 KB and 748.945 KB with a length of 2.457 KB. (30) The gene that has the name ybgd. The genomic start and stop positions of this gene are 752.018 KB and 751.452 KB with a length of 0.567 KB. (31) The gene that has the name glta and with a COG identifier of COG0372. The genomic start and stop positions of this gene are 753.691 KB and 752.408 KB with a length of 1.284 KB. (32) The gene that has the name sdhc and with a COG identifier of COG2009. The genomic start and stop positions of this gene are 754.400 KB and 756.896 KB with a length of 2.497 KB. (33) The gene that has the name sdhb and with a COG identifier of COG0479. The genomic start and stop positions of this gene are 756.912 KB and 757.628 KB with a length of 0.717 KB. (34) The gene that has the name b0725. The genomic start and stop positions of this gene are 757.687 KB and 760.730 KB with a length of 3.044 KB.

This connection controls the turning off of the expression of the following connectron(s): (1) The connectron that has the identifier 816 is on chromosome 1 in the positive direction. The genomic start and stop positions of this connectron are 707.055 KB and 707.109 KB with a length of 0.055 KB. (2) The connectron that has the identifier 819 is on chromosome 1 in the positive direction. The genomic start and stop positions of this connectron are 707.112 KB and 707.162 KB with a length of 0.051 KB. (3) The connectron that has the identifier 823 is on chromosome 1 in the negative direction. The genomic start and stop positions of this connectron are 710.647 KB and 710.633 KB with a length of 0.015 KB. (4) The connectron that has the identifier 825 is on chromosome 1 in the negative direction. The genomic start and stop positions of this connectron are 710.666 KB and 710.652 KB with a length of 0.015 KB. (5) The connectron that has the identifier 830 is on chromosome 1 in the positive direction. The genomic start and stop positions of this connectron are 714.481 KB and 714.519 KB with a length of 0.039 KB. (6) The connectron that has the identifier 832 is on chromosome 1 in the positive direction. The genomic start and stop positions of this connectron are 714.524 KB and 714.540 KB with a length of 0.017 KB. (7) The connectron that has the identifier 835 is on chromosome 1 in the negative direction. The genomic start and stop positions of this connectron are 714.593 KB and 714.543 KB with a length of 0.051 KB. (8) The connectron that has the identifier 838 is on chromosome 1 in the positive direction. The genomic start and stop positions of this connectron are 714.544 KB and 714.606 KB with a length of 0.063 KB. (9) The connectron that has the identifier 844 is on chromosome 1 in the negative direction. The genomic start and stop positions of this connectron are 720.296 KB and 720.278 KB with a length of 0.019 KB. (10) The connectron that has the identifier 852 is on chromosome 1 in the positive direction. The genomic start and stop positions of this connectron are 729.408 KB and 729.446 KB with a length of 0.039 KB. (11) The connectron that has the identifier 854 is on chromosome 1 in the positive direction. The genomic start and stop positions of this connectron are 730.443 KB and 730.457 KB with a length of 0.015 KB. (12) The connectron that has the identifier 855 is on chromosome 1 in the positive direction. The genomic start and stop positions of this connectron are 730.468 KB and 730.482 KB with a length of 0.015 KB. (13) The connectron that has the identifier 856 is on chromosome 1 in the positive direction. The genomic start and stop positions of this connectron are 730.484 KB and 730.507 KB with a length of 0.024 KB. (14) The connectron that has the identifier 857 is on chromosome 1 in the positive direction. The genomic start and stop positions of this connectron are 731.251 KB and 731.290 KB with a length of 0.040 KB. (15) The connectron that has the identifier 865 is on chromosome 1 in the positive direction. The genomic start and stop positions of this connectron are 731.994 KB and 732.016 KB with a length of 0.023 KB. The connectron that has the identifier 866 is on chromosome 1 in the positive direction. The genomic start and stop positions of this connectron are 732.019 KB and 732.034 KB with a length of 0.016 KB. (17) The connectron that has the identifier 869 is on chromosome 1 in the positive direction. The genomic start and stop positions of this connectron are 732.065 KB and 732.179 KB with a length of 0.115 KB. (18) The connectron that has the identifier 870 is on chromosome 1 in the positive direction. The genomic start and stop positions of this connectron are 732.198 KB and 732.212 KB with a length of 0.015 KB. (19) The connectron that has the identifier 872 is on chromosome 1 in the positive direction. The genomic start and stop positions of this connectron are 732.214 KB and 732.233 KB with a length of 0.020 KB. (20) The connectron that has the identifier 874 is on chromosome 1 in the positive direction. The genomic start and stop positions of this connectron are 732.235 KB and 732.283 KB with a length of 0.049 KB. (21) The connectron that has the identifier 876 is on chromosome 1 in the positive direction. The genomic start and stop positions of this connectron are 732.306 KB and 732.326 KB with a length of 0.021 KB. (22) The connectron that has the identifier 878 is on chromosome 1 in the positive direction. The genomic start and stop positions of this connectron are 732.328 KB and 732.452 KB with a length of 0.125 KB. (23) The connectron that has the identifier 880 is on chromosome 1 in the positive direction. The genomic start and stop positions of this connectron are 732.454 KB and 732.482 KB with a length of 0.029 KB. (24) The connectron that has the identifier 881 is on chromosome 1 in the positive direction. The genomic start and stop positions of this connectron are 732.488 KB and 732.519 KB with a length of 0.032 KB. (25) The connectron that has the identifier 892 is on chromosome 1 in the positive direction. The genomic start and stop positions of this connectron are 733.865 KB and 733.887 KB with a length of 0.023 KB. (26) The connectron that has the identifier 893 is on chromosome 1 in the positive direction. The genomic start and stop positions of this connectron are 733.890 KB and 733.905 KB with a length of 0.016 KB. (27) The connectron that has the identifier 895 is on chromosome 1 in the positive direction. The genomic start and stop positions of this connectron are 733.936 KB and 734.050 KB with a length of 0.115 KB. (28) The connectron that has the identifier 897 is on chromosome 1 in the positive direction. The genomic start and stop positions of this connectron are 734.052 KB and 734.139 KB with a length of 0.088 KB. (29) The connectron that has the identifier 900 is on chromosome 1 in the positive direction. The genomic start and stop positions of this connectron are 734.164 KB and 734.321 KB with a length of 0.158 KB. (30) The connectron that has the identifier 903 is on chromosome 1 in the positive direction. The genomic start and stop positions of this connectron are 734.323 KB and 734.381 KB with a length of 0.059 KB. (31) The connectron that has the identifier 904 is on chromosome 1 in the positive direction. The genomic start and stop positions of this connectron are 735.525 KB and 735.569 KB with a length of 0.045 KB. (32) The connectron that has the identifier 906 is on chromosome 1 in the positive direction. The genomic start and stop positions of this connectron are 735.606 KB and 735.703 KB with a length of 0.098 KB. (33) The connectron that has the identifier 909 is on chromosome 1 in the positive direction. The genomic start and stop positions of this connectron are 735.707 KB and 735,728 KB with a length of 0.022 KB. (34) The connectron that has the identifier 911 is on chromosome 1 in the positive direction. The genomic start and stop positions of this connectron are 735.807 KB and 735.964 KB with a length of 0.158 KB. (35) The connectron that has the identifier 915 is on chromosome 1 in the positive direction. The genomic start and stop positions of this connectron are 735.986 KB and 736.083 KB with a length of 0.098 KB. (36) The connectron that has the identifier 927 is on chromosome 1 in the positive direction. The genomic start and stop positions of this connectron are 737.017 KB and 737.032 KB with a length of 0.016 KB. (37) The connectron that has the identifier 928 is on chromosome 1 in the positive direction. The genomic start and stop positions of this connectron are 737.035 KB and 737.057 KB with a length of 0.023 KB. (38) The connectron that has the identifier 931 is on chromosome 1 in the positive direction. The genomic start and stop positions of this connectron are 737.261 KB and 737.275 KB with a length of 0.015 KB. (39) The connectron that has the identifier 932 is on chromosome 1 in the positive direction. The genomic start and stop positions of this connectron are 737.278 KB and 737.292 KB with a length of 0.015 KB. (40) The connectron that has the identifier 945 is on chromosome 1 in the positive direction. The genomic start and stop positions of this connectron are 737.930 KB and 737.967 KB with a length of 0.038 KB. (41) The connectron that has the identifier 957 is on chromosome 1 in the negative direction. The genomic start and stop positions of this connectron are 740.290 KB and 740.172 KB with a length of 0.119 KB. (42) The connectron that has the identifier 958 is on chromosome 1 in the positive direction. The genomic start and stop positions of this connectron are 740.172 KB and 740.290 KB with a length of 0.119 KB. (43) The connectron that has the identifier 965 is on chromosome 1 in the positive direction. The genomic start and stop positions of this connectron are 744.643 KB and 744.657 KB with a length of 0.015 KB. (44) The connectron that has the identifier 966 is on chromosome 1 in the positive direction. The genomic start and stop positions of this connectron are 744.664 KB and 744.678 KB with a length of 0.015 KB. (45) The connectron that has the identifier 968 is on chromosome 1 in the positive direction. The genomic start and stop positions of this connectron are 755.680 KB and 755.714 KB with a length of 0.035 KB. (46) The connectron that has the identifier 971 is on chromosome 1 in the positive direction. The genomic start and stop positions of this connectron are 757.628 KB and 757.712 KB with a length of 0.085 KB. (47) The connectron that has the identifier 974 is on chromosome 1 in the positive direction. The genomic start and stop positions of this connectron are 757.717 KB and 757.736 KB with a length of 0.020 KB.

### Addition of a Synthetic Connectron of Type 4 from Table 2

A synthetic connectron of type 4 in which the C1 and C2 are synthetic elements and the T1 and T2 are natural elements can be introduced into the E. coli genome by modifying the DNA double-strand sequences at 4626.130 kb through 4626.166 kb for a length of 0.036 kb.

Connectron 62520 is an example of a synthetic transient connectron. It is described as

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 62520 | 1 | 4651a | 4651a | 1 | 809 | 975 | transient |

The C1/C2 source of the transient connectron 62520 is represented in Table 1 as

| Type | Num | Jobno | Chr | Start | Stop | Length | GeneName |
|---|---|---|---|---|---|---|---|
| CPT | 4651a | 1 | 1 | 4626.130 | 4626.166 | .036 | \|\|\| \|\| \| \|\|\|\| \|\| \| \|\| |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB).

The T1-T2 target of the transient connectron 62520 as shown in Table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TN | 809 | 1 | 1 | 698.713 | 698.766 | .054 | *−+++*+++**++*+++++−+++++++++++ |
| GG | 810 | 1 | 1 | 698.797 | 705.113 | 6.316 | Group0149 \|\|\|\|\|\|\|\|\|\|\| \|\|\|\|\| \|\|\|\|\|\|\|\|\|\|\| |
| TN | 811 | 1 | 1 | 705.121 | 705.141 | .021 | *−++*++++++++**++++*+++++*+++++ |
| TP | 812 | 1 | 1 | 705.122 | 705.141 | .020 | *−++−+++++++++−++++++++++−+++++ |
| GG | 813 | 1 | 1 | 705.316 | 706.980 | 1.665 | Group0150 \|\| \|\|\|\|\|\|\|\|\| \|\|\|\|\|\|\|\|\|\| \|\|\|\|\| |
| TN | 814 | 1 | 1 | 707.013 | 707.028 | .016 | *−++*+++++++++*++++++++++*++++* |
| TP | 815 | 1 | 1 | 707.014 | 707.028 | .015 | *−++++++++++++++++++++++++++++− |
| CPT | 816 | 1 | 1 | 707.055 | 707.109 | .055 | −−> \|\|\|\|\|\|\|\|\|\|\|\|\|\|\|\|\|\|\|\|\|\|\|\|\|\|\|\| |
| TN | 817 | 1 | 1 | 707.055 | 707.109 | .055 | *−+++++++++*+*++++++++*+*+++++* |
| TP | 818 | 1 | 1 | 707.055 | 707.109 | .055 | *−+++++++++−+−++++++++−+−++++++ |
| CPT | 819 | 1 | 1 | 707.112 | 707.162 | .051 | OS−> \|\|\|\|\|\|\|\|\| \| \|\|\|\|\|\|\|\| \| \|\|\|\|\|\| |
| TN | 820 | 1 | 1 | 707.112 | 707.162 | .051 | *−++++*++++*+*+++++*+**+*+*+*++ |
| TP | 821 | 1 | 1 | 707.112 | 707.162 | .051 | *−++++−++++++++++++−+−++++−+−++ |
| GG | 822 | 1 | 1 | 707.557 | 710.688 | 3.131 | Group0151 \|\|\|\| \|\|\|\|\|\|\|\|\|\|\|\| \| \|\|\|\| \| \|\| |
| CNT | 823 | 1 | 1 | 710.633 | 710.647 | .015 | OS−> \|\|\|\| \|\|\|\|\|\|\|\|\|\|\|\| \| \|\|\|\| \| \|\| |
| TN | 824 | 1 | 1 | 710.633 | 710.647 | .015 | *−++++−*\|\|\|\|\|\|\|\|\|\|\| \| \|\|\|\| \| \|\| |
| CNT | 825 | 1 | 1 | 710.652 | 710.666 | .015 | −−> \|\|\|\| \|\|\|\|\|\|\|\|\|\|\| \| \|\|\|\| \| \|\| |
| GG | 826 | 1 | 1 | 710.828 | 712.755 | 1.927 | Group0152 \|\|\|\| \|\|\|\|\|\|\|\|\|\|\| \| \|\|\|\| \| \|\| |
| TN | 827 | 1 | 1 | 712.281 | 712.296 | .016 | *−++++−−+++++++++++−+−++++−+−++ |
| GG | 828 | 1 | 1 | 712.781 | 714.421 | 1.641 | Group0153 \|\|\|\| \|\|\|\|\|\|\|\|\|\|\| \| \|\|\|\| \| \|\| |
| TN | 829 | 1 | 1 | 714.481 | 714.518 | .038 | *−++++**+++*+++++++*+*++++*+*** |
| CPT | 830 | 1 | 1 | 714.481 | 714.519 | .039 | OS−> \|\|\|\|\|\|\|\|\| \|\|\|\|\|\|\|\|\|\|\|\|\|\|\|\|\| |
| TP | 831 | 1 | 1 | 714.482 | 714.519 | .038 | *−+++++++++−+++++++++++++++++−− |
| CPT | 832 | 1 | 1 | 714.524 | 714.540 | .017 | −−> \|\|\|\|\|\|\|\|\| \|\|\|\|\|\|\|\|\|\|\|\|\|\|\|\|\| |
| TN | 833 | 1 | 1 | 714.524 | 714.540 | .017 | *−+++++++++*+*+++++++++++++++** |
| TP | 834 | 1 | 1 | 714.524 | 714.540 | .017 | *−+++++++++++−+++++++++++++++++ |
| CNT | 835 | 1 | 1 | 714.543 | 714.593 | .051 | OS−> \|\|\|\|\|\|\|\|\|\|\| \|\|\|\|\|\|\|\|\|\|\|\|\|\|\|\|\| |
| TN | 836 | 1 | 1 | 714.543 | 714.593 | .051 | *−++*+++++++**++++++++*++++++++ |
| TP | 837 | 1 | 1 | 714.543 | 714.593 | .051 | *−++−+++++++−+++++++++−++++++++ |
| CPT | 838 | 1 | 1 | 714.544 | 714.606 | .063 | OS−> \|\| \|\|\|\|\|\|\| \|\|\|\|\|\|\|\|\| \|\|\|\|\|\|\|\| |
| GG | 839 | 1 | 1 | 714.635 | 715.928 | 1.293 | Group0154 \|\| \|\|\|\|\|\|\| \|\|\|\|\|\|\|\|\| \|\|\|\|\|\|\|\| |
| GN | 840 | 1 | 1 | 715.170 | 715.532 | .363 | ybfg \|\| \|\|\|\|\|\|\| \|\|\|\|\|\|\|\|\| \|\|\|\|\|\|\|\| |
| TN | 841 | 1 | 1 | 716.085 | 716.103 | .019 | *−++*+++++++*\|\|\|\|\|\|\|\|\| \|\|\|\|\|\|\|\| |
| TP | 842 | 1 | 1 | 716.085 | 716.103 | .019 | *−++++++++++++++++++++*++++++++ |
| GG | 843 | 1 | 1 | 716.169 | 724.202 | 8.033 | Group0155 \|\|\|\|\|\|\|\|\|\|\|\|\|\|\|\|\|\|\|\|\|\|\|\|\|\|\|\|\| |
| CNT | 844 | 1 | 1 | 720.278 | 720.296 | .019 | −−> \|\|\|\|\|\|\|\|\|\|\|\|\|\|\|\|\|\|\|\|\|\|\|\|\|\|\|\|\| |
| GN | 845 | 1 | 1 | 720.279 | 724.202 | 3.924 | kdpe \|\|\|\|\|\|\|\|\|\|\|\|\|\|\|\|\|\|\|\|\|\|\|\|\|\|\|\|\| |
| TP | 846 | 1 | 1 | 721.769 | 721.783 | .015 | *−+++++++++++++++++++++++++++++ |
| TN | 847 | 1 | 1 | 721.812 | 721.827 | .016 | *−+++++++++++++++++++++++++++++ |
| GG | 848 | 1 | 1 | 724.211 | 727.955 | 3.744 | Group0156 \|\|\|\|\|\|\|\|\|\|\|\|\|\|\|\|\|\|\|\|\|\|\|\|\|\|\|\|\| |
| TP | 849 | 1 | 1 | 726.665 | 726.679 | .015 | *−+++++++++++++++++++++++++++++ |
| GG | 850 | 1 | 1 | 728.357 | 733.325 | 4.968 | Group0157 \|\|\|\|\|\|\|\|\|\|\|\|\|\|\|\|\|\|\|\|\|\|\|\|\|\|\|\|\| |
| GP | 851 | 1 | 1 | 728.806 | 733.325 | 4.520 | rhsc \|\|\|\|\|\|\|\|\|\|\|\|\|\|\|\|\|\|\|\|\|\|\|\|\|\|\|\|\| |
| CPT | 852 | 1 | 1 | 729.408 | 729.446 | .039 | −−> \|\|\|\|\|\|\|\|\|\|\|\|\|\|\|\|\|\|\|\|\|\|\|\|\|\|\|\|\| |
| TP | 853 | 1 | 1 | 730.270 | 730.284 | .015 | *−+++++++++++++++++++++++++++++ |
| CPT | 854 | 1 | 1 | 730.443 | 730.457 | .015 | −−> \|\|\|\|\|\|\|\|\|\|\|\|\|\|\|\|\|\|\|\|\|\|\|\|\|\|\|\|\| |
| CPT | 855 | 1 | 1 | 730.468 | 730.482 | .015 | −−> \|\|\|\|\|\|\|\|\|\|\|\|\|\|\|\|\|\|\|\|\|\|\|\|\|\|\|\|\| |
| CPT | 856 | 1 | 1 | 730.484 | 730.507 | .024 | −−> \|\|\|\|\|\|\|\|\|\|\|\|\|\|\|\|\|\|\|\|\|\|\|\|\|\|\|\|\| |
| CPT | 857 | 1 | 1 | 731.251 | 731.290 | .040 | −−> \|\|\|\|\|\|\|\|\|\|\|\|\|\|\|\|\|\|\|\|\|\|\|\|\|\|\|\|\| |
| TP | 858 | 1 | 1 | 731.475 | 731.508 | .034 | *−+++++++++++++++++++++++++++++ |
| TP | 859 | 1 | 1 | 731.512 | 731.562 | .051 | *−+++++++++++++++++++++++++++++ |
| TP | 860 | 1 | 1 | 731.564 | 731.693 | .130 | *−+++++++++++++++++++++++++++++ |
| TP | 861 | 1 | 1 | 731.698 | 731.755 | .058 | *−+++++++++++++++++++++++++++++ |
| TP | 862 | 1 | 1 | 731.757 | 731.819 | .063 | *−+++++++++++++++++++++++++++++ |
| TP | 863 | 1 | 1 | 731.821 | 731.903 | .083 | *−+++++++++++++++++++++++++++++ |
| TP | 864 | 1 | 1 | 731.906 | 732.033 | .128 | *−+++++++++++++++++++++++++++++ |
| CPT | 865 | 1 | 1 | 731.994 | 732.016 | .023 | −−> \|\|\|\|\|\|\|\|\|\|\|\|\|\|\|\|\|\|\|\|\|\|\|\|\|\|\|\|\| |
| CPT | 866 | 1 | 1 | 732.019 | 732.034 | .016 | −−> \|\|\|\|\|\|\|\|\|\|\|\|\|\|\|\|\|\|\|\|\|\|\|\|\|\|\|\|\| |
| TP | 867 | 1 | 1 | 732.035 | 732.050 | .016 | *−+++++++++++++++++++++++++++++ |
| TP | 868 | 1 | 1 | 732.052 | 732.179 | .128 | *−+++++++++++++++++++++++++++++ |
| CPT | 869 | 1 | 1 | 732.065 | 732.179 | .115 | OS−> \|\|\|\|\|\|\|\|\|\|\|\|\|\|\|\|\|\|\|\|\|\|\|\|\|\|\|\| |
| CPT | 870 | 1 | 1 | 732.198 | 732.212 | .015 | −−> \|\|\|\|\|\|\|\|\|\|\|\|\|\|\|\|\|\|\|\|\|\|\|\|\|\|\|\| |
| TP | 871 | 1 | 1 | 732.198 | 732.212 | .015 | *−*\|\|\|\|\|\|\|\|\|\|\|\|\|\|\|\|\|\|\|\|\|\|\|\|\|\|\|\| |
| CPT | 872 | 1 | 1 | 732.214 | 732.233 | .020 | −−> \|\|\|\|\|\|\|\|\|\|\|\|\|\|\|\|\|\|\|\|\|\|\|\|\|\|\|\|\| |
| TP | 873 | 1 | 1 | 732.214 | 732.233 | .020 | *−+++++++++++++++++++++++++++++ |
| CPT | 874 | 1 | 1 | 732.235 | 732.283 | .049 | −−> \|\|\|\|\|\|\|\|\|\|\|\|\|\|\|\|\|\|\|\|\|\|\|\|\|\|\|\|\| |
| TP | 875 | 1 | 1 | 732.235 | 732.283 | .049 | *−+++++++++++++++++++++++++++++ |
| CPT | 876 | 1 | 1 | 732.306 | 732.326 | .021 | −−> \|\|\|\|\|\|\|\|\|\|\|\|\|\|\|\|\|\|\|\|\|\|\|\|\|\|\|\|\| |
| TP | 877 | 1 | 1 | 732.306 | 732.326 | .021 | *−+++++++++++++++++++++++++++++ |
| CPT | 878 | 1 | 1 | 732.328 | 732.452 | .125 | −−> \|\|\|\|\|\|\|\|\|\|\|\|\|\|\|\|\|\|\|\|\|\|\|\|\|\|\|\|\| |
| TP | 879 | 1 | 1 | 732.328 | 732.482 | .155 | *−+++++++++++++++++++++++++++++ |
| CPT | 880 | 1 | 1 | 732.454 | 732.482 | .029 | −−> \|\|\|\|\|\|\|\|\|\|\|\|\|\|\|\|\|\|\|\|\|\|\|\|\|\|\|\|\| |
| CPT | 881 | 1 | 1 | 732.488 | 732.519 | .032 | −−> \|\|\|\|\|\|\|\|\|\|\|\|\|\|\|\|\|\|\|\|\|\|\|\|\|\|\|\|\| |
| TN | 882 | 1 | 1 | 733.330 | 733.344 | .015 | *−+++++++++++++++++++++++++++++ |
| TP | 883 | 1 | 1 | 733.330 | 733.344 | .015 | *−+++++++++++++++++++++++++++++ |
| TP | 884 | 1 | 1 | 733.371 | 733.513 | .143 | *−+++++++++++++++++++++++++++++ |
| GG | 885 | 1 | 1 | 733.443 | 735.442 | 2.000 | Group0158 \|\|\|\|\|\|\|\|\|\|\|\|\|\|\|\|\|\|\|\|\|\|\|\|\|\|\|\|\| |
| TP | 886 | 1 | 1 | 733.515 | 733.564 | .050 | *−+++++++++++++++++++++++++++++ |
| TP | 887 | 1 | 1 | 733.569 | 733.626 | .058 | *−+++++++++++++++++++++++++++++ |
| TP | 888 | 1 | 1 | 733.628 | 733.645 | .018 | *−+++++++++++++++++++++++++++++ |
| TP | 889 | 1 | 1 | 733.647 | 733.696 | .050 | *−+++++++++++++++++++++++++++++ |
| TP | 890 | 1 | 1 | 733.698 | 733.774 | .077 | *−+++++++++++++++++++++++++++++ |
| TP | 891 | 1 | 1 | 733.776 | 733.904 | .129 | *−+++++++++++++++++++++++++++++ |
| CPT | 892 | 1 | 1 | 733.865 | 733.887 | .023 | OS−> \|\|\|\|\|\|\|\|\|\|\|\|\|\|\|\|\|\|\|\|\|\|\|\|\|\|\|\| |
| CPT | 893 | 1 | 1 | 733.890 | 733.905 | .016 | −−> \|\|\|\|\|\|\|\|\|\|\|\|\|\|\|\|\|\|\|\|\|\|\|\|\|\|\|\|\| |
| TP | 894 | 1 | 1 | 733.906 | 734.050 | .145 | *−+*+++++++++++++++++++++++++++ |
| CPT | 895 | 1 | 1 | 733.936 | 734.050 | .115 | −−> \| \|\|\|\|\|\|\|\|\|\|\|\|\|\|\|\|\|\|\|\|\|\|\|\|\|\|\| |
| TP | 896 | 1 | 1 | 734.052 | 734.075 | .024 | *−+−+++++++++++++++++++++++++++ |
| CPT | 897 | 1 | 1 | 734.052 | 734.139 | .088 | −−> \| \|\|\|\|\|\|\|\|\|\|\|\|\|\|\|\|\|\|\|\|\|\|\|\|\|\|\| |
| TP | 898 | 1 | 1 | 734.084 | 734.139 | .056 | *−*−+++++++++++++++++++++++++++ |
| TP | 899 | 1 | 1 | 734.149 | 734.179 | .031 | *−−−+++++++++++++++++++++++++++ |
| CPT | 900 | 1 | 1 | 734.164 | 734.321 | .158 | −−> \|\|\|\|\|\|\|\|\|\|\|\|\|\|\|\|\|\|\|\|\|\|\|\|\|\|\| |
| TP | 901 | 1 | 1 | 734.192 | 734.321 | .130 | *−−−+++++++++++++++++++++++++++ |
| TP | 902 | 1 | 1 | 734.323 | 734.378 | .056 | *−−−+++++++++++++++++++++++++++ |
| CPT | 903 | 1 | 1 | 734.323 | 734.381 | .059 | −−> \|\|\|\|\|\|\|\|\|\|\|\|\|\|\|\|\|\|\|\|\|\|\|\|\|\|\| |
| CPT | 904 | 1 | 1 | 735.525 | 735.569 | .045 | −−> \|\|\|\|\|\|\|\|\|\|\|\|\|\|\|\|\|\|\|\|\|\|\|\|\|\|\| |
| TP | 905 | 1 | 1 | 735.525 | 735.569 | .045 | *−* \|\|\|\|\|\|\|\|\|\|\|\|\|\|\|\|\|\|\|\|\|\|\|\|\|\|\| |
| CPT | 906 | 1 | 1 | 735.606 | 735.703 | .098 | −−> \| \|\|\|\|\|\|\|\|\|\|\|\|\|\|\|\|\|\|\|\|\|\|\|\|\|\|\| |
| TP | 907 | 1 | 1 | 735.606 | 735.703 | .098 | *−+*+++++++++++++++++++++++++++ |
| TP | 908 | 1 | 1 | 735.707 | 735.721 | .015 | *−+++++++++++++++++++++++++++++ |
| CPT | 909 | 1 | 1 | 735.707 | 735.728 | .022 | −−> \|\|\|\|\|\|\|\|\|\|\|\|\|\|\|\|\|\|\|\|\|\|\|\|\|\|\|\|\| |
| TP | 910 | 1 | 1 | 735.749 | 735.803 | .055 | *−+++++++++++++++++++++++++++++ |
| CPT | 911 | 1 | 1 | 735.807 | 735.964 | .158 | OS−> \|\|\|\|\|\|\|\|\|\|\|\|\|\|\|\|\|\|\|\|\|\|\|\|\|\|\|\|\| |
| TP | 912 | 1 | 1 | 735.814 | 735.895 | .082 | *−+++++++++++++++++++++++++++++ |
| TP | 913 | 1 | 1 | 735.905 | 735.934 | .030 | *−+++++++++++++++++++++++++++++ |
| TP | 914 | 1 | 1 | 735.938 | 736.083 | .146 | *−+++++++++++++++++++++++++++++ |
| CPT | 915 | 1 | 1 | 735.986 | 736.083 | .098 | OS−> \|\|\|\|\|\|\|\|\|\|\|\|\|\|\|\|\|\|\|\|\|\|\|\|\|\|\|\|\| |
| TP | 916 | 1 | 1 | 736.152 | 736.173 | .022 | *−+++++++++++++++++++++++++++++ |
| TP | 917 | 1 | 1 | 736.187 | 736.217 | .031 | *−+++++++++++++++++++++++++++++ |
| TP | 918 | 1 | 1 | 736.219 | 736.265 | .047 | *−+++++++++++++++++++++++++++++ |
| TP | 919 | 1 | 1 | 736.288 | 736.334 | .047 | *−+++++++++++++++++++++++++++++ |
| GG | 920 | 1 | 1 | 736.327 | 737.184 | .858 | Group0159 \|\|\|\|\|\|\|\|\|\|\|\|\|\|\|\|\|\|\|\|\|\|\|\|\|\|\|\|\| |
| TP | 921 | 1 | 1 | 736.349 | 736.469 | .221 | *−+++++++++++++++++++++++++++++ |
| TP | 922 | 1 | 1 | 736.516 | 736.628 | .113 | *−+++++++++++++++++++++++++++++ |
| TP | 923 | 1 | 1 | 736.630 | 736.645 | .016 | *−+++++++++++++++++++++++++++++ |
| TP | 924 | 1 | 1 | 736.654 | 736.792 | .139 | *−+++++++++++++++++++++++++++++ |
| TN | 925 | 1 | 1 | 736.654 | 736.809 | .156 | *−+++++++++++++++++++++++++++++ |
| TN | 926 | 1 | 1 | 736.816 | 736.832 | .017 | *−+++++++++++++++++++++++++++++ |
| CPT | 927 | 1 | 1 | 737.017 | 737.032 | .016 | −−> \|\|\|\|\|\|\|\|\|\|\|\|\|\|\|\|\|\|\|\|\|\|\|\|\|\|\|\|\| |
| CPT | 928 | 1 | 1 | 737.035 | 737.057 | .023 | −−> \|\|\|\|\|\|\|\|\|\|\|\|\|\|\|\|\|\|\|\|\|\|\|\|\|\|\|\|\| |
| TN | 929 | 1 | 1 | 737.168 | 737.188 | .021 | *−+++++++++++++++++++++++++++++ |
| TP | 930 | 1 | 1 | 737.196 | 737.290 | .095 | *−**+++++++++++++++++++++++++++ |
| CPT | 931 | 1 | 1 | 737.261 | 737.275 | .015 | −−> \|\|\|\|\|\|\|\|\|\|\|\|\|\|\|\|\|\|\|\|\|\|\|\|\|\|\| |
| CPT | 932 | 1 | 1 | 737.278 | 737.292 | .015 | −−> \|\|\|\|\|\|\|\|\|\|\|\|\|\|\|\|\|\|\|\|\|\|\|\|\|\|\| |
| TP | 933 | 1 | 1 | 737.292 | 737.393 | .102 | *−−−+++++++++++++++++++++++++++ |
| GG | 934 | 1 | 1 | 737.315 | 738.076 | .762 | Group0160 \|\|\|\|\|\|\|\|\|\|\|\|\|\|\|\|\|\|\|\|\|\|\|\|\|\|\| |
| TP | 935 | 1 | 1 | 737.411 | 737.475 | .065 | *−−−+++++++++++++++++++++++++++ |
| TP | 936 | 1 | 1 | 737.479 | 737.557 | .079 | *−−−+++++++++++++++++++++++++++ |
| TP | 937 | 1 | 1 | 737.580 | 737.597 | .018 | *−−−+++++++++++++++++++++++++++ |
| TP | 938 | 1 | 1 | 737.605 | 737.626 | .022 | *−−−+++++++++++++++++++++++++++ |
| TP | 939 | 1 | 1 | 737.633 | 737.648 | .016 | *−−−+++++++++++++++++++++++++++ |
| TP | 940 | 1 | 1 | 737.653 | 737.679 | .027 | *−−−+++++++++++++++++++++++++++ |
| TP | 941 | 1 | 1 | 737.683 | 737.752 | .070 | *−−−+++++++++++++++++++++++++++ |
| TP | 942 | 1 | 1 | 737.798 | 737.822 | .025 | *−−−+++++++++++++++++++++++++++ |
| TP | 943 | 1 | 1 | 737.828 | 737.895 | .068 | *−−−+++++++++++++++++++++++++++ |
| TP | 944 | 1 | 1 | 737.897 | 737.931 | .035 | *−−−+++++++++++++++++++++++++++ |
| CPT | 945 | 1 | 1 | 737.930 | 737.967 | .038 | −−> \|\|\|\|\|\|\|\|\|\|\|\|\|\|\|\|\|\|\|\|\|\|\|\|\|\|\| |
| TP | 946 | 1 | 1 | 737.936 | 737.967 | .032 | *−−−+++++++++++++++++++++++++++ |
| TP | 947 | 1 | 1 | 737.970 | 737.988 | .019 | *−−−+++++++++++++++++++++++++++ |
| TN | 948 | 1 | 1 | 737.990 | 738.015 | .026 | *−−−+++++++++++++++++++++++++++ |
| TP | 949 | 1 | 1 | 737.990 | 738.015 | .026 | *−−−+++++++++++++++++++++++++++ |
| TN | 950 | 1 | 1 | 738.019 | 738.051 | .033 | *−−−+++++++++++++++++++++++++++ |
| TP | 951 | 1 | 1 | 738.019 | 738.051 | .033 | *−−−+++++++++++++++++++++++++++ |
| TP | 952 | 1 | 1 | 738.053 | 738.074 | .022 | *−−−+++++++++++++++++++++++++++ |
| TN | 953 | 1 | 1 | 738.058 | 738.074 | .017 | *−−−+++++++++++++++++++++++++++ |
| TN | 954 | 1 | 1 | 738.107 | 738.126 | .020 | *−−−+++++++++++++++++++++++++++ |
| GG | 955 | 1 | 1 | 738.224 | 740.148 | 1.925 | Group0161 \|\|\|\|\|\|\|\|\|\|\|\|\|\|\|\|\|\|\|\|\|\|\|\|\|\|\| |
| TN | 956 | 1 | 1 | 740.172 | 740.213 | .042 | *−**++++*++++*+++*++++++*++++++ |
| CNT | 957 | 1 | 1 | 740.172 | 740.290 | .119 | OS−> \|\|\|\|\|\| \|\|\|\| \|\|\| \|\|\|\|\|\| \|\|\|\|\|\| |
| CPT | 958 | 1 | 1 | 740.172 | 740.290 | .119 | OS−> \|\|\|\|\|\| \|\|\|\| \|\|\| \|\|\|\|\|\| \|\|\|\|\|\| |
| TP | 959 | 1 | 1 | 740.173 | 740.213 | .041 | *−++++++*++++*+++**+++++*++++++ |
| TP | 960 | 1 | 1 | 740.216 | 740.241 | .026 | *−++++++++++++++++−++++++++++++ |
| TN | 961 | 1 | 1 | 740.216 | 740.288 | .073 | *−+++*++++++++**++−++++++++++++ |
| TP | 962 | 1 | 1 | 740.244 | 740.288 | .045 | *−+++−++++++++−−++*+++**+++++++ |
| GG | 963 | 1 | 1 | 740.298 | 757.628 | 17.330 | Group0162 \|\|\| \|\|\|\|\|\|\|\| \|\|\|\|\|\| \|\|\|\|\|\|\| |
| GP | 964 | 1 | 1 | 742.816 | 745.122 | 2.307 | ybgj \|\|\| \|\|\|\|\|\|\|\| \|\|\|\|\|\| \|\|\|\|\|\|\| |
| CPT | 965 | 1 | 1 | 744.643 | 744.657 | .015 | −−> \|\|\| \|\|\|\|\|\|\|\| \|\|\|\|\|\| \|\|\|\|\|\|\| |
| CPT | 966 | 1 | 1 | 744.664 | 744.678 | .015 | −−> \|\|\| \|\|\|\|\|\|\|\| \|\|\|\|\|\| \|\|\|\|\|\|\| |
| GP | 967 | 1 | 1 | 754.400 | 756.896 | 2.497 | sdhc \|\|\| \|\|\|\|\|\|\|\| \|\|\|\|\|\| \|\|\|\|\|\|\| |
| CPT | 968 | 1 | 1 | 755.680 | 755.714 | .035 | −−> \|\|\| \|\|\|\|\|\|\|\| \|\|\|\|\|\| \|\|\|\|\|\|\| |
| GP | 969 | 1 | 1 | 756.912 | 757.628 | .717 | sdhb \|\|\| \|\|\|\|\|\|\|\| \|\|\|\|\|\| \|\|\|\|\|\|\| |
| TN | 970 | 1 | 1 | 757.626 | 757.712 | .087 | *−*+**+++*++++***+++++**+++*+++ |
| CPT | 971 | 1 | 1 | 757.628 | 757.712 | .085 | OS−> \| \|\|\|\| \|\|\|\|\|\| \|\|\|\|\|\|\|\|\|\| \|\|\| |
| TP | 972 | 1 | 1 | 757.629 | 757.712 | .084 | *−−+−+++*−++++++−+*++++++++−+++ |
| GG | 973 | 1 | 1 | 757.687 | 760.730 | 3.044 | Group0163 \| \|\|\| \|\|\|\|\|\| \| \|\|\|\|\|\|\|\| \|\|\| |
| CPT | 974 | 1 | 1 | 757.717 | 757.736 | .020 | −−> \| \|\|\| \|\|\|\|\|\| \| \|\|\|\|\|\|\|\| \|\|\| |
| TN | 975 | 1 | 1 | 757.718 | 757.753 | .036 | *−*+*+++***+++++*+−+*++++++−+++ |

The “Type” descriptor of the T1 (Id number 809) is “TN” showing that is T1 target is on the negative strand of the double-stranded genomic DNA. Because the T1 and the T2 targets have to be on the same strand, the T2 target (Id number 975) also has the type descriptor “TN”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is changed to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Group0149 through Group0163 are described in Table 2 as

| Gene_Name | COG_Id | | Chromosome | Direction | Start | Stop | Length |
|---|---|---|---|---|---|---|---|
| Group0149 | | | | | | | |
| nagd | COG0647 | b | 1 | negative | 698.797 | 699.549 | .753 |
| nagc | COG1940 | b | 1 | negative | 699.597 | 700.817 | 1.221 |
| naga | COG1820 | b | 1 | negative | 700.826 | 701.974 | 1.149 |
| nagb | COG0363 | b | 1 | negative | 702.034 | 702.834 | .801 |
| nage | COG1264 | b | 1 | positive | 703.167 | 705.113 | 1.947 |
| Group0150 | | | | | | | |
| gins | COG0008 | b | 1 | positive | 705.316 | 706.980 | 1.665 |
| Group0151 | | | | | | | |
| ybfm | — | b | 1 | positive | 707.557 | 708.963 | 1.407 |
| ybfn | — | b | 1 | positive | 709.013 | 709.339 | .327 |
| fur | COG0735 | b | 1 | negative | 709.423 | 709.869 | .447 |
| flda | COG0716 | b | 1 | negative | 710.158 | 710.688 | .531 |
| Group0152 | | | | | | | |
| ybfe | — | b | 1 | negative | 710.828 | 711.190 | .363 |
| ybff | COG0596 | b | 1 | negative | 711.261 | 712.025 | .765 |
| seqa | COG3057 | b | 1 | positive | 712.210 | 712.755 | .546 |
| Group0153 | | | | | | | |
| pgm | COG0033 | b | 1 | positive | 712.781 | 714.421 | 1.641 |
| Group0154 | | | | | | | |
| ybfp | — | b | 1 | positive | 714.635 | 715.129 | .495 |
| ybfg | — | b | 1 | negative | 715.170 | 715.532 | .363 |
| ybfh | — | b | 1 | negative | 715.611 | 715.928 | .318 |
| Group0155 | | | | | | | |
| pote | COG0531 | b | 1 | negative | 716.169 | 719.683 | 3.515 |
| kdpe | COG0745 | b | 1 | negative | 720.279 | 724.202 | 3.924 |
| Group0156 | | | | | | | |
| kdpb | COG2216 | b | 1 | negative | 724.211 | 726.259 | 2.049 |
| kdpa | COG2060 | b | 1 | negative | 726.282 | 727.955 | 1.674 |
| Group0157 | | | | | | | |
| ybfa | — | b | 1 | positive | 728.357 | 728.563 | .207 |
| rhsc | — | b | 1 | positive | 728.806 | 733.325 | 4.520 |
| Group0158 | | | | | | | |
| b0703 | COG3209 | b | 1 | positive | 733.443 | 735.442 | 2.000 |
| Group0159 | | | | | | | |
| ybfl | — | b | 1 | positive | 736.327 | 737.184 | .858 |
| Group0160 | | | | | | | |
| ybfd | — | b | 1 | positive | 737.315 | 738.076 | .762 |
| Group0161 | | | | | | | |
| ybga | COG3272 | b | 1 | positive | 738.224 | 740.148 | 1.925 |
| Group0162 | | | | | | | |
| ybgh | COG3104 | b | 1 | negative | 740.298 | 741.779 | 1.482 |
| ybgi | COG0327 | b | 1 | positive | 742.050 | 742.793 | .744 |
| ybgj | COG2049 | b | 1 | positive | 742.816 | 745.122 | 2.307 |
| nei | COG0266 | b | 1 | positive | 745.158 | 745.949 | .792 |
| abrb | — | b | 1 | negative | 745.946 | 747.037 | 1.092 |
| ybgo | — | b | 1 | negative | 747.144 | 748.930 | 1.787 |
| ybgq | — | b | 1 | negative | 748.945 | 751.401 | 2.457 |
| ybgd | — | b | 1 | negative | 751.452 | 752.018 | .567 |
| glta | COG0372 | b | 1 | negative | 752.408 | 753.691 | 1.284 |
| sdhc | COG2009 | b | 1 | positive | 754.400 | 756.896 | 2.497 |
| sdhb | COG0479 | b | 1 | positive | 756.912 | 757.628 | .717 |
| Group0163 | | | | | | | |
| b0725 | — | b | 1 | positive | 757.687 | 760.730 | 3.044 |

All of the data for the transient connectron 62520 are pulled together in the following table that is the “terse” description of the connectron.

| Connectron Relationships - | Global_Id | Type |
|---|---|---|
| | 62520 | transient |

| | | Chro- | | | | |
|---|---|---|---|---|---|---|
| Control | | mo- | C1/ | | | |
| Sequences - | Direction | some | C2_Id | Start | Stop | Length |
| | positive | 1 | 4651a | 4626.130 | 4626.166 | .036 |

| Trigger Gene - | Name | COG_Id | Start | Stop | Length |
|---|---|---|---|---|---|
| | sms | | 4623.481 | 4626.116 | 2.636 |

| | | Chro- | | | | |
|---|---|---|---|---|---|---|
| Target | | mo- | | | | |
| Sequences | Direction | some | | Start | Stop | Length |
| | | | T1_Id | | | |
| | negative | 1 | 809 | 698.766 | 698.713 | .054 |
| | | | T2_Id | | | |
| | | | 975 | 757.753 | 757.718 | .036 |

Controlled Genes

| Local_Id | Chromosome | Group | Name | COG_Id | Direction | Start | Stop | Length |
|---|---|---|---|---|---|---|---|---|
| 1 | 1 | Group0149 | nagd | COG0647 | positive | 698.797 | 705.113 | 6.316 |
| 2 | 1 | Group0149 | nagc | COG1940 | negative | 700.817 | 699.597 | 1.221 |
| 3 | 1 | Group0149 | naga | COG1820 | negative | 701.974 | 700.826 | 1.149 |
| 4 | 1 | Group0149 | nagb | COG0363 | negative | 702.834 | 702.034 | .801 |
| 5 | 1 | Group0149 | nage | COG1264 | positive | 703.167 | 705.113 | 1.947 |
| 6 | 1 | Group0150 | gins | COG0008 | positive | 705.316 | 706.980 | 1.665 |
| 7 | 1 | Group0151 | ybfm | — | positive | 707.557 | 710.688 | 3.131 |
| 8 | 1 | Group0151 | ybfn | — | positive | 709.013 | 709.339 | .327 |
| 9 | 1 | Group0151 | fur | COG0735 | negative | 709.869 | 709.423 | .447 |
| 10 | 1 | Group0151 | flda | COG0716 | negative | 710.688 | 710.158 | .531 |
| 11 | 1 | Group0152 | ybfe | — | positive | 710.828 | 712.755 | 1.927 |
| 12 | 1 | Group0152 | ybff | COG0596 | negative | 712.025 | 711.261 | .765 |
| 13 | 1 | Group0152 | seqa | COG3057 | positive | 712.210 | 712.755 | .546 |
| 14 | 1 | Group0153 | pgm | COG0033 | positive | 712.781 | 714.421 | 1.641 |
| 15 | 1 | Group0154 | ybfp | — | positive | 714.635 | 715.928 | 1.293 |
| 16 | 1 | Group0154 | ybfg | — | negative | 715.532 | 715.170 | .363 |
| 17 | 1 | Group0154 | ybfh | — | negative | 715.928 | 715.611 | .318 |
| 18 | 1 | Group0155 | pote | COG0531 | positive | 716.169 | 724.202 | 8.033 |
| 19 | 1 | Group0155 | kdpe | COG0745 | negative | 724.202 | 720.279 | 3.924 |
| 20 | 1 | Group0156 | kdpb | COG2216 | positive | 724.211 | 727.955 | 3.744 |
| 21 | 1 | Group0156 | kdpa | COG2060 | negative | 727.955 | 726.282 | 1.674 |
| 22 | 1 | Group0157 | ybfa | — | positive | 728.357 | 733.325 | 4.968 |
| 23 | 1 | Group0157 | rhsc | — | positive | 728.806 | 733.325 | 4.520 |
| 24 | 1 | Group0158 | b0703 | COG3209 | positive | 733.443 | 735.442 | 2.000 |
| 25 | 1 | Group0159 | ybfl | — | positive | 736.327 | 737.184 | .858 |
| 26 | 1 | Group0160 | ybfd | — | positive | 737.315 | 738.076 | .762 |
| 27 | 1 | Group0161 | ybga | COG3272 | positive | 738.224 | 740.148 | 1.925 |
| 28 | 1 | Group0162 | ybgh | COG3104 | positive | 740.298 | 757.628 | 17.330 |
| 29 | 1 | Group0162 | ybgi | COG0327 | positive | 742.050 | 742.793 | .744 |
| 30 | 1 | Group0162 | ybgj | COG2049 | positive | 742.816 | 745.122 | 2.307 |
| 31 | 1 | Group0162 | nei | COG0266 | positive | 745.158 | 745.949 | .792 |
| 32 | 1 | Group0162 | abrb | — | negative | 747.037 | 745.946 | 1.092 |
| 33 | 1 | Group0162 | ybgo | — | negative | 748.930 | 747.144 | 1.787 |
| 34 | 1 | Group0162 | ybgq | — | negative | 751.401 | 748.945 | 2.457 |
| 35 | 1 | Group0162 | ybgd | — | negative | 752.018 | 751.452 | .567 |
| 36 | 1 | Group0162 | glta | COG0372 | negative | 753.691 | 752.408 | 1.284 |
| 37 | 1 | Group0162 | sdhc | COG2009 | positive | 754.400 | 756.896 | 2.497 |
| 38 | 1 | Group0162 | sdhb | COG0479 | positive | 756.912 | 757.628 | .717 |
| 39 | 1 | Group0163 | b0725 | — | positive | 757.687 | 760.730 | 3.044 |

Controlled Connectrons

| | Chro- | | | | | |
|---|---|---|---|---|---|---|
| Local_{—} | mo- | C1/ | Direc- | | | |
| Id | some | C2_Id | tion | Start | Stop | Length |
| 1 | 1 | 816 | positive | 707.055 | 707.109 | .055 |
| 2 | 1 | 819 | positive | 707.112 | 707.162 | .051 |
| 3 | 1 | 823 | negative | 710.647 | 710.633 | .015 |
| 4 | 1 | 825 | negative | 710.666 | 710.652 | .015 |
| 5 | 1 | 830 | positive | 714.481 | 714.519 | .039 |
| 6 | 1 | 832 | positive | 714.524 | 714.540 | .017 |
| 7 | 1 | 835 | negative | 714.593 | 714.543 | .051 |
| 8 | 1 | 838 | positive | 714.544 | 714.606 | .063 |
| 9 | 1 | 844 | negative | 720.296 | 720.278 | .019 |
| 10 | 1 | 852 | positive | 729.408 | 729.446 | .039 |
| 11 | 1 | 854 | positive | 730.443 | 730.457 | .015 |
| 12 | 1 | 855 | positive | 730.468 | 730.482 | .015 |
| 13 | 1 | 856 | positive | 730.484 | 730.507 | .024 |
| 14 | 1 | 857 | positive | 731.251 | 731.290 | .040 |
| 15 | 1 | 865 | positive | 731.994 | 732.016 | .023 |
| 16 | 1 | 866 | positive | 732.019 | 732.034 | .016 |
| 17 | 1 | 869 | positive | 732.065 | 732.179 | .115 |
| 18 | 1 | 870 | positive | 732.198 | 732.212 | .015 |
| 19 | 1 | 872 | positive | 732.214 | 732.233 | .020 |
| 20 | 1 | 874 | positive | 732.235 | 732.283 | .049 |
| 21 | 1 | 876 | positive | 732.306 | 732.326 | .021 |
| 22 | 1 | 878 | positive | 732.328 | 732.452 | .125 |
| 23 | 1 | 880 | positive | 732.454 | 732.482 | .029 |
| 24 | 1 | 881 | positive | 732.488 | 732.519 | .032 |
| 25 | 1 | 892 | positive | 733.865 | 733.887 | .023 |
| 26 | 1 | 893 | positive | 733.890 | 733.905 | .016 |
| 27 | 1 | 895 | positive | 733.936 | 734.050 | .115 |
| 28 | 1 | 897 | positive | 734.052 | 734.139 | .088 |
| 29 | 1 | 900 | positive | 734.164 | 734.321 | .158 |
| 30 | 1 | 903 | positive | 734.323 | 734.381 | .059 |
| 31 | 1 | 904 | positive | 735.525 | 735.569 | .045 |
| 32 | 1 | 906 | positive | 735.606 | 735.703 | .098 |
| 33 | 1 | 909 | positive | 735.707 | 735.728 | .022 |
| 34 | 1 | 911 | positive | 735.807 | 735.964 | .158 |
| 35 | 1 | 915 | positive | 735.986 | 736.083 | .098 |
| 36 | 1 | 927 | positive | 737.017 | 737.032 | .016 |
| 37 | 1 | 928 | positive | 737.035 | 737.057 | .023 |
| 38 | 1 | 931 | positive | 737.261 | 737.275 | .015 |
| 39 | 1 | 932 | positive | 737.278 | 737.292 | .015 |
| 40 | 1 | 945 | positive | 737.930 | 737.967 | .038 |
| 41 | 1 | 957 | negative | 740.290 | 740.172 | .119 |
| 42 | 1 | 958 | positive | 740.172 | 740.290 | .119 |
| 43 | 1 | 965 | positive | 744.643 | 744.657 | .015 |
| 44 | 1 | 966 | positive | 744.664 | 744.678 | .015 |
| 45 | 1 | 968 | positive | 755.680 | 755.714 | .035 |
| 46 | 1 | 971 | positive | 757.628 | 757.712 | .085 |
| 47 | 1 | 974 | positive | 757.717 | 757.736 | .020 |

The verbose description of the synthetic connectron 62520 is:

In the Escherichia coli K-12 MG1655 complete genome the transient connectron number 62520 is generated by the control sequence (C1/C2) whose identifier number is 4651a. This control sequence is on the positive strand of the genomic DNA of chromosome 1. The genomic start and stop positions of this control sequence are 4626.130 KB and 4626.166 KB with a length of 0.036 KB. Expression of the RNA for this connectron is triggered by the promotion of the gene whose name is sms. The genomic start and stop positions of this gene are 4623.481 KB and 4626.116 KB and with a length of 2.636 KB. This connectron causes stabilization of a loop of DNA. The target sequences (T1-T2) are on the negative strand of the genomic DNA on chromosome 1. The identifier number of the initiating target sequence (T1) is 809. The genomic start and stop positions of this initiating target sequence are 698.766 KB and 698.713 KB with a length of 0.054 KB. The identifier number of the terminating target sequence (T2) is 975. The genomic start and stop positions of this terminating target sequence are 757.753 KB and 757.718 KB with a length of 0.036 KB.

This connectron controls the modulation of the expression of the following gene(s): (1) The gene that has the name nagd and with a COG identifier of COG0647. The genomic start and stop positions of this gene are 698.797 KB and 705.113 KB with a length of 6.316 KB. (2) The gene that has the name nagc and with a COG identifier of COG1940. The genomic start and stop positions of this gene are 700.817 KB and 699.597 KB with a length of 1.221 KB. (3) The gene that has the name naga and with a COG identifier of COG1820. The genomic start and stop positions of this gene are 701.974 KB and 700.826 KB with a length of 1.149 KB. (4) The gene that has the name nagb and with a COG identifier of COG0363. The genomic start and stop positions of this gene are 702.834 KB and 702.034 KB with a length of 0.801 KB. (5) The gene that has the name nage and with a COG identifier of COG1264. The genomic start and stop positions of this gene are 703.167 KB and 705.113 KB with a length of 1.947 KB. (6) The gene that has the name gins and with a COG identifier of COG0008. The genomic start and stop positions of this gene are 705.316 KB and 706.980 KB with a length of 1.665 KB. (7) The gene that has the name ybfm. The genomic start and stop positions of this gene are 707.557 KB and 710.688 KB with a length of 3.131 KB. (8) The gene that has the name ybfn. The genomic start and stop positions of this gene are 709.013 KB and 709.339 KB with a length of 0.327 KB. (9) The gene that has the name fur and with a COG identifier of COG0735. The genomic start and stop positions of this gene are 709.869 KB and 709.423 KB with a length of 0.447 KB. (10) The gene that has the name flda and with a COG identifier of COG0716. The genomic start and stop positions of this gene are 710.688 KB and 710.158 KB with a length of 0.531 KB. (11) The gene that has the name ybfe. The genomic start and stop positions of this gene are 710.828 KB and 712.755 KB with a length of 1.927 KB. (12) The gene that has the name ybff and with a COG identifier of COG0596. The genomic start and stop positions of this gene are 712.025 KB and 711.261 KB with a length of 0.765 KB. (13) The gene that has the name seqa and with a COG identifier of COG3057. The genomic start and stop positions of this gene are 712.210 KB and 712.755 KB with a length of 0.546 KB. (14) The gene that has the name pgm and with a COG identifier of COG0033. The genomic start and stop positions of this gene are 712.781 KB and 714.421 KB with a length of 1.641 KB. (15) The gene that has the name ybfp. The genomic start and stop positions of this gene are 714.635 KB and 715.928 KB with a length of 1.293 KB. (16) The gene that has the name ybfg. The genomic start and stop positions of this gene are 715.532 KB and 715.170 KB with a length of 0.363 KB. (17) The gene that has the name ybfh. The genomic start and stop positions of this gene are 715.928 KB and 715.611 KB with a length of 0.318 KB. (18) The gene that has the name pote and with a COG identifier of COG0531. The genomic start and stop positions of this gene are 716.169 KB and 724.202 KB with a length of 8.033 KB. (19) The gene that has the name kdpe and with a COG identifier of COG0745. The genomic start and stop positions of this gene are 724.202 KB and 720.279 KB with a length of 3.924 KB. (20) The gene that has the name kdpb and with a COG identifier of COG2216. The genomic start and stop positions of this gene are 724.211 KB and 727.955 KB with a length of 3.744 KB. (21) The gene that has the name kdpa and with a COG identifier of COG2060. The genomic start and stop positions of this gene are 727.955 KB and 726.282 KB with a length of 1.674 KB. (22) The gene that has the name ybfa. The genomic start and stop positions of this gene are 728.357 KB and 733.325 KB with a length of 4.968 KB. (23) The gene that has the name rhsc. The genomic start and stop positions of this gene are 728.806 KB and 733.325 KB with a length of 4.520 KB. (24) The gene that has the name b0703 and with a COG identifier of COG3209. The genomic start and stop positions of this gene are 733.443 KB and 735.442 KB with a length of 2.000 KB. (25) The gene that has the name ybfl. The genomic start and stop positions of this gene are 736.327 KB and 737.184 KB with a length of 0.858 KB. (26) The gene that has the name ybfd. The genomic start and stop positions of this gene are 737.315 KB and 738.076 KB with a length of 0.762 KB. (27) The gene that has the name ybga and with a COG identifier of COG3272. The genomic start and stop positions of this gene are 738.224 KB and 740.148 KB with a length of 1.925 KB. (28) The gene that has the name ybgh and with a COG identifier of COG3104. The genomic start and stop positions of this gene are 740.298 KB and 757.628 KB with a length of 17.330 KB. (29) The gene that has the name ybgi and with a COG identifier of COG0327. The genomic start and stop positions of this gene are 742.050 KB and 742.793 KB with a length of 0.744 KB. (30) The gene that has the name ybgj and with a COG identifier of COG2049. The genomic start and stop positions of this gene are 742.816 KB and 745.122 KB with a length of 2.307 KB. (31) The gene that has the name nei and with a COG identifier of COG0266. The genomic start and stop positions of this gene are 745.158 KB and 745.949 KB with a length of 0.792 KB. (32) The gene that has the name abrb. The genomic start and stop positions of this gene are 747.037 KB and 745.946 KB with a length of 1.092 KB. (33) The gene that has the name ybgo. The genomic start and stop positions of this gene are 748.930 KB and 747.144 KB with a length of 1.787 KB. (34) The gene that has the name ybgq. The genomic start and stop positions of this gene are 751.401 KB and 748.945 KB with a length of 2.457 KB. (35) The gene that has the name ybgd. The genomic start and stop positions of this gene are 752.018 KB and 751.452 KB with a length of 0.567 KB. (36) The gene that has the name glta and with a COG identifier of COG0372. The genomic start and stop positions of this gene are 753.691 KB and 752.408 KB with a length of 1.284 KB. (37) The gene that has the name sdhc and with a COG identifier of COG2009. The genomic start and stop positions of this gene are 754.400 KB and 756.896 KB with a length of 2.497 KB. (38) The gene that has the name sdhb and with a COG identifier of COG0479. The genomic start and stop positions of this gene are 756.912 KB and 757.628 KB with a length of 0.717 KB. (39) The gene that has the name b0725. The genomic start and stop positions of this gene are 757.687 KB and 760.730 KB with a length of 3.044 KB.

This connectron controls the turning off of the expression of the following connectron(s): (1) The connectron that has the identifier 816 is on chromosome 1 in the positive direction. The genomic start and stop positions of this connectron are 707.055 KB and 707.109 KB with a length of 0.055 KB. (2) The connectron that has the identifier 819 is on chromosome 1 in the positive direction. The genomic start and stop positions of this connectron are 707.112 KB and 707.162 KB with a length of 0.051 KB. (3) The connectron that has the identifier 823 is on chromosome 1 in the negative direction. The genomic start and stop positions of this connectron are 710.647 KB and 710.633 KB with a length of 0.015 KB. (4) The connectron that has the identifier 825 is on chromosome 1 in the negative direction. The genomic start and stop positions of this connectron are 710.666 KB and 710.652 KB with a length of 0.015 KB. (5) The connectron that has the identifier 830 is on chromosome 1 in the positive direction. The genomic start and stop positions of this connectron are 714.481 KB and 714.519 KB with a length of 0.039 KB. (6) The connectron that has the identifier 832 is on chromosome 1 in the positive direction. The genomic start and stop positions of this connectron are 714.524 KB and 714.540 KB with a length of 0.017 KB. (7) The connectron that has the identifier 835 is on chromosome 1 in the negative direction. The genomic start and stop positions of this connectron are 714.593 KB and 714.543 KB with a length of 0.051 KB. (8) The connectron that has the identifier 838 is on chromosome 1 in the positive direction. The genomic start and stop positions of this connectron are 714.544 KB and 714.606 KB with a length of 0.063 KB. (9) The connectron that has the identifier 844 is on chromosome 1 in the negative direction. The genomic start and stop positions of this connectron are 720.296 KB and 720.278 KB with a length of 0.019 KB. (10) The connectron that has the identifier 852 is on chromosome 1 in the positive direction. The genomic start and stop positions of this connectron are 729.408 KB and 729.446 KB with a length of 0.039 KB. (11) The connectron that has the identifier 854 is on chromosome 1 in the positive direction. The genomic start and stop positions of this connectron are 730.443 KB and 730.457 KB with a length of 0.015 KB. (12) The connectron that has the identifier 855 is on chromosome 1 in the positive direction. The genomic start and stop positions of this connectron are 730.468 KB and 730.482 KB with a length of 0.015 KB. (13) The connectron that has the identifier 856 is on chromosome 1 in the positive direction. The genomic start and stop positions of this connectron are 730.484 KB and 730.507 KB with a length of 0.024 KB. (14) The connectron that has the identifier 857 is on chromosome 1 in the positive direction. The genomic start and stop positions of this connectron are 731.251 KB and 731.290 KB with a length of 0.040 KB. (15) The connectron that has the identifier 865 is on chromosome 1 in the positive direction. The genomic start and stop positions of this connectron are 731.994 KB and 732.016 KB with a length of 0.023 KB. (16) The connectron that has the identifier 866 is on chromosome 1 in the positive direction. The genomic start and stop positions of this connectron are 732.019 KB and 732.034 KB with a length of 0.016 KB. (17) The connectron that has the identifier 869 is on chromosome 1 in the positive direction. The genomic start and stop positions of this connectron are 732.065 KB and 732.179 KB with a length of 0.115 KB. (18) The connectron that has the identifier 870 is on chromosome 1 in the positive direction. The genomic start and stop positions of this connectron are 732.198 KB and 732.212 KB with a length of 0.015 KB. (19) The connectron that has the identifier 872 is on chromosome 1 in the positive direction. The genomic start and stop positions of this connectron are 732.214 KB and 732.233 KB with a length of 0.020 KB. (20) The connectron that has the identifier 874 is on chromosome 1 in the positive direction. The genomic start and stop positions of this connectron are 732.235 KB and 732.283 KB with a length of 0.049 KB. (21) The connectron that has the identifier 876 is on chromosome 1 in the positive direction. The genomic start and stop positions of this connectron are 732.306 KB and 732.326 KB with a length of 0.021 KB. (22) The connectron that has the identifier 878 is on chromosome 1 in the positive direction. The genomic start and stop positions of this connectron are 732.328 KB and 732.452 KB with a length of 0.125 KB. (23) The connectron that has the identifier 880 is on chromosome 1 in the positive direction. The genomic start and stop positions of this connectron are 732.454 KB and 732.482 KB with a length of 0.029 KB. (24) The connectron that has the identifier 881 is on chromosome 1 in the positive direction. The genomic start and stop positions of this connectron are 732.488 KB and 732.519 KB with a length of 0.032 KB. (25) The connectron that has the identifier 892 is on chromosome 1 in the positive direction. The genomic start and stop positions of this connectron are 733.865 KB and 733.887 KB with a length of 0.023 KB. (26) The connectron that has the identifier 893 is on chromosome 1 in the positive direction. The genomic start and stop positions of this connectron are 733.890 KB and 733.905 KB with a length of 0.016 KB. (27) The connectron that has the identifier 895 is on chromosome 1 in the positive direction. The genomic start and stop positions of this connectron are 733.936 KB and 734.050 KB with a length of 0.115 KB. (28) The connectron that has the identifier 897 is on chromosome 1 in the positive direction. The genomic start and stop positions of this connectron are 734.052 KB and 734.139 KB with a length of 0.088 KB. (29) The connectron that has the identifier 900 is on chromosome 1 in the positive direction. The genomic start and stop positions of this connectron are 734.164 KB and 734.321 KB with a length of 0.158 KB. (30) The connectron that has the identifier 903 is on chromosome 1 in the positive direction. The genomic start and stop positions of this connectron are 734.323 KB and 734.381 KB with a length of 0.059 KB. (31) The connectron that has the identifier 904 is on chromosome 1 in the positive direction. The genomic start and stop positions of this connectron are 735.525 KB and 735.569 KB with a length of 0.045 KB. (32) The connectron that has the identifier 906 is on chromosome 1 in the positive direction. The genomic start and stop positions of this connectron are 735.606 KB and 735.703 KB with a length of 0.098 KB. (33) The connectron that has the identifier 909 is on chromosome 1 in the positive direction. The genomic start and stop positions of this connectron are 735.707 KB and 735.728 KB with a length of 0.022 KB. (34) The connectron that has the identifier 911 is on chromosome 1 in the positive direction. The genomic start and stop positions of this connectron are 735.807 KB and 735.964 KB with a length of 0.158 KB. (35) The connectron that has the identifier 915 is on chromosome 1 in the positive direction. The genomic start and stop positions of this connectron are 735.986 KB and 736.083 KB with a length of 0.098 KB. (36) The connectron that has the identifier 927 is on chromosome 1 in the positive direction. The genomic start and stop positions of this connectron are 737.017 KB and 737.032 KB with a length of 0.016 KB. (37) The connectron that has the identifier 928 is on chromosome 1 in the positive direction. The genomic start and stop positions of this connectron are 737.035 KB and 737.057 KB with a length of 0.023 KB. (38) The connectron that has the identifier 931 is on chromosome 1 in the positive direction. The genomic start and stop positions of this connectron are 737.261 KB and 737.275 KB with a length of 0.015 KB. (39) The connectron that has the identifier 932 is on chromosome 1 in the positive direction. The genomic start and stop positions of this connectron are 737.278 KB and 737.292 KB with a length of 0.015 KB. (40) The connectron that has the identifier 945 is on chromosome 1 in the positive direction. The genomic start and stop positions of this connectron are 737.930 KB and 737.967 KB with a length of 0.038 KB. (41) The connectron that has the identifier 957 is on chromosome 1 in the negative direction. The genomic start and stop positions of this connectron are 740.290 KB and 740.172 KB with a length of 0.119 KB. (42) The connectron that has the identifier 958 is on chromosome 1 in the positive direction. The genomic start and stop positions of this connectron are 740.172 KB and 740.290 KB with a length of 0.119 KB. (43) The connectron that has the identifier 965 is on chromosome 1 in the positive direction. The genomic start and stop positions of this connectron are 744.643 KB and 744.657 KB with a length of 0.015 KB. (44) The connectron that has the identifier 966 is on chromosome 1 in the positive direction. The genomic start and stop positions of this connectron are 744.664 KB and 744.678 KB with a length of 0.015 KB. (45) The connectron that has the identifier 968 is on chromosome 1 in the positive direction. The genomic start and stop positions of this connectron are 755.680 KB and 755.714 KB with a length of 0.035 KB. (46) The connectron that has the identifier 971 is on chromosome 1 in the positive direction. The genomic start and stop positions of this connectron are 757.628 KB and 757.712 KB with a length of 0.085 KB. (47) The connectron that has the identifier 974 is on chromosome 1 in the positive direction. The genomic start and stop positions of this connectron are 757.717 KB and 757.736 KB with a length of 0.020 KB.

### Addition of a Synthetic Transient Connectron Between Chromosomes—Type 7 from Table 2

A synthetic virtual connectron of type 7 in which the C1 is a synthetic element and C2 is a natural element, the T1 is a natural element and the T2 is a synthetic element can be introduced into the S. cerevisiae genome by modifying the DNA double-strand sequence at 221.330 kb through 221.345 kb on chromosome 2 for a length of 0.016 kb and by modifying the DNA double-strand sequence at 488.140 kb through 488.297 on chromosome 5 for a length of 0.158 kb.

To create this example of a synthetic connectron of type 7 the T2 sequence for natural connectron 3558 (old position 497.581 kb through 498.091 kb) was copied into the new position stated above.

Connectron 75273 is an example of a transient connectron. It is described as

| | | C1/C2 | | | T1-T2 | | |
|---|---|---|---|---|---|---|---|
| Global_Id | Chromosome | C1_Id | C2_Id | Chromosome | T1_ld | T2_Id | Connectron_Type |
| 75273 | 2 | 792a | 793 | 5 | 4749 | 4824a | transient |

The C1/C2 source of the transient connectron 75273 is represented in table 1 as

| Type | Num | Jobno | Chr | Start | Stop | Length | GeneName |
|---|---|---|---|---|---|---|---|
| CPT | 792a | 22 | 2 | 221.330 | 221.345 | .016 | --> |
| CPT | 793 | 22 | 2 | 221.346 | 221.361 | .016 | --> |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB). The symbol OS-> occurs because there is another connectron formed by this C1/C2 that happens to be a one-shot connectron.

The T1-T2 target of the transient connectron 75273 as shown in table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TN | 4749 | 59 | 5 | 448.454 | 448.992 | .539 | *−* |
| TP | 4750 | 68 | 5 | 448.454 | 448.992 | .539 | *−+ |
| CNT | 4751 | 64 | 5 | 448.454 | 448.992 | .539 | −−> \| |
| TP | 4752 | 63 | 5 | 449.003 | 449.315 | .313 | *−+ |
| TN | 4753 | 65 | 5 | 449.003 | 449.315 | .313 | *−+ |
| CNT | 4754 | 60 | 5 | 449.003 | 449.315 | .313 | OS−> \| |
| CNT | 4755 | 5 | 5 | 449.317 | 449.482 | .166 | OS−> \| |
| TN | 4756 | 5 | 5 | 449.317 | 449.482 | .166 | *−+ |
| TP | 4757 | 64 | 5 | 449.317 | 449.482 | .148 | *−+ |
| GG | 4758 | 59 | 5 | 449.470 | 449.574 | .105 | Group0615 \| |
| CPT | 4759 | 70 | 5 | 449.490 | 449.563 | .074 | −−> \| |
| TN | 4760 | 70 | 5 | 449.490 | 449.563 | .074 | *−+ |
| TP | 4761 | 67 | 5 | 449.490 | 449.558 | .069 | *−+ |
| CNT | 4762 | 70 | 5 | 449.490 | 449.563 | .074 | −−> \| |
| TN | 4763 | 59 | 5 | 449.575 | 449.603 | .029 | *−+ |
| TP | 4764 | 59 | 5 | 449.575 | 449.603 | .029 | *−+ |
| GG | 4765 | 59 | 5 | 450.558 | 453.230 | 2.672 | Group0616 \| |
| TP | 4766 | 59 | 5 | 452.176 | 452.190 | .015 | *−+ |
| GG | 4767 | 59 | 5 | 453.454 | 454.914 | 1.461 | Group0617 \| |
| CNT | 4768 | 20 | 5 | 453.909 | 453.923 | .015 | −−> \| |
| CNT | 4769 | 20 | 5 | 453.928 | 453.942 | .015 | −−> \| |
| TP | 4770 | 59 | 5 | 454.644 | 454.658 | .015 | *−+ |
| GG | 4771 | 59 | 5 | 455.141 | 457.600 | 2.460 | Group0618 \| |
| TN | 4772 | 66 | 5 | 456.316 | 456.331 | .016 | *−+ |
| GG | 4773 | 59 | 5 | 457.801 | 460.218 | 2.418 | Group0619 \| |
| TN | 4774 | 66 | 5 | 460.340 | 460.355 | .016 | *−+ |
| GG | 4775 | 59 | 5 | 460.521 | 466.020 | 5.500 | Group0620 \| |
| GP | 4776 | 59 | 5 | 462.580 | 462.861 | .282 | LSM5 \| |
| TN | 4777 | 59 | 5 | 463.743 | 463.758 | .016 | *−+ |
| TP | 4778 | 59 | 5 | 463.743 | 463.758 | .016 | *−+ |
| CPT | 4779 | 61 | 5 | 463.990 | 464.004 | .015 | −−> \| |
| CPT | 4780 | 61 | 5 | 464.010 | 464.024 | .015 | −−> \| |
| TP | 4781 | 59 | 5 | 465.739 | 465.754 | .016 | *−\| |
| GG | 4782 | 59 | 5 | 466.203 | 468.811 | 2.609 | Group0621 \| |
| GP | 4783 | 59 | 5 | 468.365 | 468.811 | .447 | SPI1 \| |
| TP | 4784 | 69 | 5 | 469.451 | 469.518 | .059 | *−+ |
| CNT | 4785 | 67 | 5 | 469.451 | 469.530 | .080 | OS−> \| |
| GG | 4786 | 59 | 5 | 469.452 | 469.525 | .074 | Group0622 \| |
| TN | 4787 | 59 | 5 | 469.452 | 469.532 | .081 | *−+ |
| CPT | 4788 | 67 | 5 | 469.452 | 469.531 | .080 | −−> \| |
| GG | 4789 | 59 | 5 | 469.681 | 472.419 | 2.739 | Group0623 \| |
| TN | 4790 | 20 | 5 | 471.772 | 471.786 | .015 | *−+ |
| TP | 4791 | 20 | 5 | 471.772 | 471.786 | .015 | *−+ |
| GG | 4792 | 59 | 5 | 472.652 | 482.843 | 10.192 | Group0624 \| |
| GP | 4793 | 59 | 5 | 475.015 | 476.223 | 1.209 | OXA1 \| |
| TP | 4794 | 63 | 5 | 476.803 | 476.817 | .015 | *−+ |
| CPT | 4795 | 60 | 5 | 478.412 | 478.426 | .015 | −−> \| |
| CPT | 4796 | 60 | 5 | 478.436 | 478.450 | .015 | −−> \| |
| CNT | 4797 | 47 | 5 | 481.782 | 481.812 | .031 | −−> \| |
| TN | 4798 | 20 | 5 | 482.192 | 482.206 | .015 | *−+ |
| CNT | 4799 | 67 | 5 | 482.676 | 482.690 | .015 | −−> \| |
| CNT | 4800 | 67 | 5 | 482.701 | 482.715 | .015 | −−> \| |
| GG | 4801 | 59 | 5 | 483.320 | 487.188 | 3.869 | Group0625 \| |
| GP | 4802 | 59 | 5 | 484.783 | 487.188 | 2.406 | SEC34 \| |
| CNT | 4803 | 61 | 5 | 485.099 | 485.113 | .015 | −−> \| |
| CNT | 4804 | 61 | 5 | 485.119 | 485.134 | .016 | −−> \| |
| TN | 4805 | 64 | 5 | 487.041 | 487.056 | .016 | *−+ |
| GG | 4806 | 59 | 5 | 487.326 | 487.397 | .072 | Group0626 \| |
| CPT | 4807 | 61 | 5 | 487.375 | 487.397 | .023 | −−> \| |
| CPT | 4808 | 61 | 5 | 487.400 | 487.415 | .016 | −−> \| |
| CNT | 4809 | 34 | 5 | 487.830 | 487.848 | .019 | OS−> \| |
| CPT | 4810 | 62 | 5 | 487.830 | 487.848 | .019 | OS−> \| |
| TP | 4811 | 60 | 5 | 487.830 | 487.848 | .019 | *−+ |
| TN | 4812 | 59 | 5 | 487.830 | 487.848 | .019 | *−+ |
| CNT | 4813 | 34 | 5 | 487.850 | 488.016 | .167 | OS−> \| |
| CPT | 4814 | 5 | 5 | 487.850 | 488.016 | .167 | OS−> \| |
| TP | 4815 | 5 | 5 | 487.850 | 488.016 | .167 | *−+ |
| TN | 4816 | 64 | 5 | 487.850 | 488.016 | .167 | *−+ |
| CNT | 4817 | 47 | 5 | 488.024 | 488.137 | .114 | OS−> \| |
| CPT | 4818 | 47 | 5 | 488.024 | 488.068 | .045 | −−> \| |
| TN | 4819 | 34 | 5 | 488.024 | 488.067 | .044 | *−+ |
| TP | 4820 | 34 | 5 | 488.024 | 488.067 | .044 | *−+ |
| CPT | 4821 | 69 | 5 | 488.069 | 488.137 | .069 | OS−> \| |
| TN | 4822 | 67 | 5 | 488.069 | 488.122 | .054 | *−+ |
| TP | 4823 | 59 | 5 | 488.069 | 488.085 | .017 | *−+ |
| TP | 4824 | 5 | 5 | 488.094 | 488.137 | .044 | *−+ |
| TN | 4824a | 5 | 5 | 488.140 | 488.297 | .158 | *−* |

The “Type” descriptor of the Ti (Id number 4749) is “TN” showing that is T1 target is on the negative strand of the double-stranded genomic DNA. Because the T1 and the T2 targets have to be on the same strand, the T2 target (Id number 4824a) also has the type descriptor “TN”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is changed to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Group0615 (Id number 4758) has the type descriptor “GG”. The same is true for Group0626 (Id number 4806).

Group0615 through Group0626 are described in table 2 as

| Gene_Name | COG_Id | | Chromosome | Direction | Start | Stop | Length |
|---|---|---|---|---|---|---|---|
| Group0615 | | | | | | | |
| YER138W-A | — | | 5 | positive | 449.470 | 449.574 | .105 |
| Group0616 | | | | | | | |
| YER139C | — | | 5 | negative | 450.558 | 451.238 | .681 |
| YER140W | — | | 5 | positive | 451.560 | 453.230 | 1.671 |
| Group0617 | | | | | | | |
| COX15 | COG1612 | O | 5 | positive | 453.454 | 454.914 | 1.461 |
| Group0618 | | | | | | | |
| MAG1 | COG0122 | L | 5 | negative | 455.141 | 456.031 | .891 |
| DDI1 | — | | 5 | positive | 456.314 | 457.600 | 1.287 |
| Group0619 | | | | | | | |
| UBP5 | — | | 5 | negative | 457.801 | 460.218 | 2.418 |
| Group0620 | | | | | | | |
| FTR1 | COG0672 | P | 5 | negative | 460.521 | 461.735 | 1.215 |
| LSM5 | COG1958 | K | 5 | positive | 462.580 | 462.861 | .282 |
| YER147C | — | | 5 | negative | 462.963 | 464.837 | 1.875 |
| SPT15 | COG2101 | K | 5 | positive | 465.298 | 466.020 | .723 |
| Group0621 | | | | | | | |
| PEA2 | — | | 5 | negative | 466.203 | 467.465 | 1.263 |
| SPI1 | — | | 5 | positive | 468.365 | 468.811 | .447 |
| Group0622 | | | | | | | |
| | | | 5 | positive | 469.452 | 469.525 | .074 |
| Group0623 | | | | | | | |
| UBP3 | — | | 5 | negative | 469.681 | 472.419 | 2.739 |
| Group0624 | | | | | | | |
| YER152C | COG1167 | K | 5 | negative | 472.652 | 473.983 | 1.332 |
| PET122 | — | | 5 | negative | 474.036 | 474.800 | .765 |
| OXA1 | COG0706 | N | 5 | positive | 475.015 | 476.223 | 1.209 |
| BEM2 | — | | 5 | negative | 476.340 | 482.843 | 6.504 |
| Group0625 | | | | | | | |
| YER156C | — | | 5 | negative | 483.320 | 484.336 | 1.017 |
| SEC34 | — | | 5 | positive | 484.783 | 487.188 | 2.406 |
| Group0626 | | | | | | | |
| | | | 5 | positive | 487.326 | 487.397 | .072 |

All of the data for the transient connectron 75273 are pulled together in the following table that is the “terse” description of the connectron.

| Connectron Relationships - | Global_Id | Type |
|---|---|---|
| | 75273 | transient |

| | | Chro- | | | | |
|---|---|---|---|---|---|---|
| Control | | mo- | C1/ | | | |
| Sequences - | Direction | some | C2_Id | Start | Stop | Length |
| | positive | 2 | 792a | 221.330 | 221.345 | .016 |
| | positive | 2 | 793 | 221.346 | 221.361 | .016 |

| Trigger Gene - | Name | COG_Id | Start | Stop | Length |
|---|---|---|---|---|---|
| | YBL005W-B | — | 221.293 | 226.561 | 5.269 |

| | | Chro- | | | | |
|---|---|---|---|---|---|---|
| Target | | mo- | | | | |
| Sequences - | Direction | some | | Start | Stop | Length |
| | | | T1_Id | | | |
| | negative | 5 | 4749 | 448.992 | 448.454 | .539 |
| | | | T2_Id | | | |
| | | | 4824a | 488.140 | 488.297 | .158 |

Controlled Genes

| Local_Id | Chromosome | Group | Name | COG_Id | Direction | Start | Stop | Length |
|---|---|---|---|---|---|---|---|---|
| 1 | 5 | Group0615 | YER138W-A | — | positive | 449.470 | 449.574 | .105 |
| 2 | 5 | Group0616 | YER139C | — | positive | 450.558 | 453.230 | 2.673 |
| 3 | 5 | Group0616 | YER140W | — | positive | 451.560 | 453.230 | 1.671 |
| 4 | 5 | Group0617 | COX15 | COG1612 | positive | 453.454 | 454.914 | 1.461 |
| 5 | 5 | Group0618 | MAG1 | COG0122 | positive | 455.141 | 457.600 | 2.459 |
| 6 | 5 | Group0618 | DDI1 | — | positive | 456.314 | 457.600 | 1.287 |
| 7 | 5 | Group0619 | UBP5 | — | positive | 457.801 | 460.218 | 2.418 |
| 8 | 5 | Group0620 | FTR1 | COG0672 | positive | 460.521 | 466.020 | 5.499 |
| 9 | 5 | Group0620 | LSM5 | COG1958 | positive | 462.580 | 462.861 | .282 |
| 10 | 5 | Group0620 | YER147C | — | negative | 464.837 | 462.963 | 1.875 |
| 11 | 5 | Group0620 | SPT15 | COG2101 | positive | 465.298 | 466.020 | .723 |
| 12 | 5 | Group0621 | PEA2 | — | positive | 466.203 | 468.811 | 2.608 |
| 13 | 5 | Group0621 | SPI1 | — | positive | 468.365 | 468.811 | .447 |
| 14 | 5 | Group0622 | | | positive | 469.452 | 469.525 | .074 |
| 15 | 5 | Group0623 | UBP3 | — | positive | 469.681 | 472.419 | 2.739 |
| 16 | 5 | Group0624 | YER152C | COG1167 | positive | 472.652 | 482.843 | 10.191 |
| 17 | 5 | Group0624 | PET122 | — | negative | 474.800 | 474.036 | .765 |
| 18 | 5 | Group0624 | OXA1 | COG0706 | positive | 475.015 | 476.223 | 1.209 |
| 19 | 5 | Group0624 | BEM2 | — | negative | 482.843 | 476.340 | 6.504 |
| 20 | 5 | Group0625 | YER156C | — | positive | 483.320 | 487.188 | 3.868 |
| 21 | 5 | Group0625 | SEC34 | — | positive | 484.783 | 487.188 | 2.406 |
| 22 | 5 | Group0626 | | | positive | 487.326 | 487.397 | .072 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 5 | 4751 | negative | 448.992 | 448.454 | .539 |
| 2 | 5 | 4754 | negative | 449.315 | 449.003 | .313 |
| 3 | 5 | 4755 | negative | 449.482 | 449.317 | .166 |
| 4 | 5 | 4759 | positive | 449.490 | 449.563 | .074 |
| 5 | 5 | 4762 | negative | 449.563 | 449.490 | .074 |
| 6 | 5 | 4768 | negative | 453.923 | 453.909 | .015 |
| 7 | 5 | 4769 | negative | 453.942 | 453.928 | .015 |
| 8 | 5 | 4779 | positive | 463.990 | 464.004 | .015 |
| 9 | 5 | 4780 | positive | 464.010 | 464.024 | .015 |
| 10 | 5 | 4785 | negative | 469.530 | 469.451 | .080 |
| 11 | 5 | 4788 | positive | 469.452 | 469.531 | .080 |
| 12 | 5 | 4795 | positive | 478.412 | 478.426 | .015 |
| 13 | 5 | 4796 | positive | 478.436 | 478.450 | .015 |
| 14 | 5 | 4797 | negative | 481.812 | 481.782 | .031 |
| 15 | 5 | 4799 | negative | 482.690 | 482.676 | .015 |
| 16 | 5 | 4800 | negative | 482.715 | 482.701 | .015 |
| 17 | 5 | 4803 | negative | 485.113 | 485.099 | .015 |
| 18 | 5 | 4804 | negative | 485.134 | 485.119 | .016 |
| 19 | 5 | 4807 | positive | 487.375 | 487.397 | .023 |
| 20 | 5 | 4808 | positive | 487.400 | 487.415 | .016 |
| 21 | 5 | 4809 | negative | 487.848 | 487.830 | .019 |
| 22 | 5 | 4810 | positive | 487.830 | 487.848 | .019 |
| 23 | 5 | 4813 | negative | 488.016 | 487.850 | .167 |
| 24 | 5 | 4814 | positive | 487.850 | 488.016 | .167 |
| 25 | 5 | 4817 | negative | 488.137 | 488.024 | .114 |
| 26 | 5 | 4818 | positive | 488.024 | 488.068 | .045 |
| 27 | 5 | 4821 | positive | 488.069 | 488.137 | .069 |

The verbose description of the transient connectron 75273 is:

In the Saccharomyces cerevisiae complete genome the transient connectron number 75273 is generated by the control sequence C1 whose identifier number is 792a. This control sequence is on the positive strand of the genomic DNA of chromosome 2. The genomic start and stop positions of this control sequence are 221.330 KB and 221.345 KB with a length of 0.016 KB and by the control sequence C2 whose identifier number is 793. This control sequence is on the positive strand of the genomic DNA of chromosome 2. The genomic start and stop positions of this control sequence are 221.346 KB and 221.361 KB with a length of 0.016 KB. Expression of the RNA for this connectron is triggered by the promotion of the gene whose name is YBL005W-B. The genomic start and stop positions of this gene are 221.293 KB and 226.561 KB and with a length of 5.269 KB. This connectron causes stabilization of a loop of DNA. The target sequences (T1-T2) are on the negative strand of the genomic DNA on chromosome 5. The identifier number of the initiating target sequence (T1) is 4749. The genomic start and stop positions of this initiating target sequence are 448.992 KB and 448.454 KB with a length of 0.022 KB. The identifier number of the terminating target sequence (T2) is 4824a. The genomic start and stop positions of this terminating target sequence are 488.140 KB and 488.297 KB with a length of 0.158 KB.

This connectron controls the modulation of the expression of the following gene(s): (1) The gene that has the name YER138W-A. The genomic start and stop positions of this gene are 449.470 KB and 449.574 KB with a length of 0.105 KB. (2) The gene that has the name YER139C. The genomic start and stop positions of this gene are 450.558 KB and 453.230 KB with a length of 2.672 KB. (3) The gene that has the name YER140W. The genomic start and stop positions of this gene are 451.560 KB and 453.230 KB with a length of 1.671 KB. (4) The gene that has the name COX15 and with a COG identifier of COG1612. The genomic start and stop positions of this gene are 453.454 KB and 454.914 KB with a length of 1.461 KB. (5) The gene that has the name MAGi and with a COG identifier of COG0122. The genomic start and stop positions of this gene are 455.141 KB and 457.600 KB with a length of 2.459 KB. (6) The gene that has the name DDI1. The genomic start and stop positions of this gene are 456.314 KB and 457.600 KB with a length of 1.287 KB. (7) The gene that has the name UBP5. The genomic start and stop positions of this gene are 457.801 KB and 460.218 KB with a length of 2.418 KB. (8) The gene that has the name FTR1 and with a COG identifier of COG0672. The genomic start and stop positions of this gene are 460.521 KB and 466.020 KB with a length of 5.499 KB. (9) The gene that has the name LSM5 and with a COG identifier of COG1958. The genomic start and stop positions of this gene are 462.580 KB and 462.861 KB with a length of 0.282 KB. (10) The gene that has the name YER147C. The genomic start and stop positions of this gene are 464.837 KB and 462.963 KB with a length of 1.875 KB. (11) The gene that has the name SPT15 and with a COG identifier of COG2101. The genomic start and stop positions of this gene are 465.298 KB and 466.020 KB with a length of 0.723 KB. (12) The gene that has the name PEA2. The genomic start and stop positions of this gene are 466.203 KB and 468.811 KB with a length of 2.608 KB. (13) The gene that has the name SPI1. The genomic start and stop positions of this gene are 468.365 KB and 468.811 KB with a length of 0.447 KB. (14) The gene that has the name No_Name. The genomic start and stop positions of this gene are 469.452 KB and 469.525 KB with a length of 0.074 KB. (15) The gene that has the name UBP3. The genomic start and stop positions of this gene are 469.681 KB and 472.419 KB with a length of 2.739 KB. (16) The gene that has the name YER152C and with a COG identifier of COG1167. The genomic start and stop positions of this gene are 472.652 KB and 482.843 KB with a length of 10.191 KB. (17) The gene that has the name PET122. The genomic start and stop positions of this gene are 474.800 KB and 474.036 KB with a length of 0.765 KB. (18) The gene that has the name OXA1 and with a COG identifier of COG0706. The genomic start and stop positions of this gene are 475.015 KB and 476.223 KB with a length of 1.209 KB. (19) The gene that has the name BEM2. The genomic start and stop positions of this gene are 482.843 KB and 476.340 KB with a length of 6.504 KB. (20) The gene that has the name YER156C. The genomic start and stop positions of this gene are 483.320 KB and 487.188 KB with a length of 3.868 KB. (21) The gene that has the name SEC34. The genomic start and stop positions of this gene are 484.783 KB and 487.188 KB with a length of 2.406 KB. (22) The gene that has the name No_Name. The genomic start and stop positions of this gene are 487.326 KB and 487.397 KB with a length of 0.072 KB.

This connectron controls the turning off of the expression of the following connectron(s): (1) The connectron that has the identifier 4751 is on chromosome 5 in the negative direction. The genomic start and stop positions of this connectron are 448.992 KB and 448.454 KB with a length of 0.539 KB. (2) The connectron that has the identifier 4754 is on chromosome 5 in the negative direction. The genomic start and stop positions of this connectron are 449.315 KB and 449.003 KB with a length of 0.313 KB. (3) The connectron that has the identifier 4755 is on chromosome 5 in the negative direction. The genomic start and stop positions of this connectron are 449.482 KB and 449.317 KB with a length of 0.166 KB. (4) The connectron that has the identifier 4759 is on chromosome 5 in the positive direction. The genomic start and stop positions of this connectron are 449.490 KB and 449.563 KB with a length of 0.074 KB. (5) The connectron that has the identifier 4762 is on chromosome 5 in the negative direction. The genomic start and stop positions of this connectron are 449.563 KB and 449.490 KB with a length of 0.074 KB. (6) The connectron that has the identifier 4768 is on chromosome 5 in the negative direction. The genomic start and stop positions of this connectron are 453.923 KB and 453.909 KB with a length of 0.015 KB. (7) The connectron that has the identifier 4769 is on chromosome 5 in the negative direction. The genomic start and stop positions of this connectron are 453.942 KB and 453.928 KB with a length of 0.015 KB. (8) The connectron that has the identifier 4779 is on chromosome 5 in the positive direction. The genomic start and stop positions of this connectron are 463.990 KB and 464.004 KB with a length of 0.015 KB. (9) The connectron that has the identifier 4780 is on chromosome 5 in the positive direction. The genomic start and stop positions of this connectron are 464.010 KB and 464.024 KB with a length of 0.015 KB. (10) The connectron that has the identifier 4785 is on chromosome 5 in the negative direction. The genomic start and stop positions of this connectron are 469.530 KB and 469.451 KB with a length of 0.080 KB. (11) The connectron that has the identifier 4788 is on chromosome 5 in the positive direction. The genomic start and stop positions of this connectron are 469.452 KB and 469.531 KB with a length of 0.080 KB. (12) The connectron that has the identifier 4795 is on chromosome 5 in the positive direction. The genomic start and stop positions of this connectron are 478.412 KB and 478.426 KB with a length of 0.015 KB. (13) The connectron that has the identifier 4796 is on chromosome 5 in the positive direction. The genomic start and stop positions of this connectron are 478.436 KB and 478.450 KB with a length of 0.015 KB. (14) The connectron that has the identifier 4797 is on chromosome 5 in the negative direction. The genomic start and stop positions of this connectron are 481.812 KB and 481.782 KB with a length of 0.031 KB. (15) The connectron that has the identifier 4799 is on chromosome 5 in the negative direction. The genomic start and stop positions of this connectron are 482.690 KB and 482.676 KB with a length of 0.015 KB. (16) The connectron that has the identifier 4800 is on chromosome 5 in the negative direction. The genomic start and stop positions of this connectron are 482.715 KB and 482.701 KB with a length of 0.015 KB. (17) The connectron that has the identifier 4803 is on chromosome 5 in the negative direction. The genomic start and stop positions of this connectron are 485.113 KB and 485.099 KB with a length of 0.015 KB. (18) The connectron that has the identifier 4804 is on chromosome 5 in the negative direction. The genomic start and stop positions of this connectron are 485.134 KB and 485.119 KB with a length of 0.016 KB. (19) The connectron that has the identifier 4807 is on chromosome 5 in the positive direction. The genomic start and stop positions of this connectron are 487.375 KB and 487.397 KB with a length of 0.023 KB. (20) The connectron that has the identifier 4808 is on chromosome 5 in the positive direction. The genomic start and stop positions of this connectron are 487.400 KB and 487.415 KB with a length of 0.016 KB. (21) The connectron that has the identifier 4809 is on chromosome 5 in the negative direction. The genomic start and stop positions of this connectron are 487.848 KB and 487.830 KB with a length of 0.019 KB. (22) The connectron that has the identifier 4810 is on chromosome 5 in the positive direction. The genomic start and stop positions of this connectron are 487.830 KB and 487.848 KB with a length of 0.019 KB. (23) The connectron that has the identifier 4813 is on chromosome 5 in the negative direction. The genomic start and stop positions of this connectron are 488.016 KB and 487.850 KB with a length of 0.167 KB. (24) The connectron that has the identifier 4814 is on chromosome 5 in the positive direction. The genomic start and stop positions of this connectron are 487.850 KB and 488.016 KB with a length of 0.022 KB. (25) The connectron that has the identifier 4817 is on chromosome 5 in the negative direction. The genomic start and stop positions of this connectron are 488.137 KB and 488.024 KB with a length of 0.114 KB. (26) The connectron that has the identifier 4818 is on chromosome 5 in the positive direction. The genomic start and stop positions of this connectron are 488.024 KB and 488.068 KB with a length of 0.045 KB. (27) The connectron that has the identifier 4821 is on chromosome 5 in the positive direction. The genomic start and stop positions of this connectron are 488.069 KB and 488.137 KB with a length of 0.069 KB.

### Addition of a Synthetic Connectron of Type 14 from Table 2

A synthetic virtual connectron of type 14 in which the C1 and C2 are natural elements, the T1 is a synthetic element and the T2 is a natual element can be introduced into the Halobacterium sp. genome by modifying the DNA double-strand sequences at 734.750 kb through 734.942 kb for a length of 0.193 kb.

To create this example of a synthetic connectron of type 15 the T1 sequence for natural connectron 6627 (old position 733.018 kb through 733.210 kb) was copied into the new position stated above.

Connectron 7341 is an example of a transient connectron. It is described as

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 2587 | 1 | 6612 | 6612 | 1 | 6627 | 6852 | transient |

The C1/C2 source of the transient connectron 7341 is represented in Table 1 as

| Type | Num | Jobno | Chr | Start | Stop | Length | GeneName |
|---|---|---|---|---|---|---|---|
| CPT | 6612 | 1 | 1 | 732.401 | 732.534 | .134 | OS−> \|\|\|\|\|\|\|\|\|\|\|\|\|\|\|\|\|\|\|\|\|\|\|\|\|\|\|\|\| |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB). The symbol OS-> occurs because there is another connectron formed by this C1/C2 that happens to be a one-shot connectron.

The T1-T2 target of the transient connectron 7341 as shown in Table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TP | 6644a | 1 | 1 | 734.750 | 734.942 | .193 | *−* |
| GG | 6646 | 1 | 1 | 735.009 | 735.881 | .873 | Group0493 \| |
| CNT | 6647 | 1 | 1 | 735.364 | 735.378 | .015 | OS−> \| |
| TN | 6648 | 1 | 1 | 735.364 | 735.378 | .015 | *−+ |
| CNT | 6649 | 1 | 1 | 735.389 | 735.403 | .015 | −−> \| |
| CNT | 6650 | 1 | 1 | 735.753 | 735.767 | .015 | −−> \| |
| CNT | 6651 | 1 | 1 | 735.775 | 735.789 | .015 | −−> \| |
| GG | 6652 | 1 | 1 | 735.910 | 737.544 | 1.634 | Group0494 \| |
| TN | 6653 | 1 | 1 | 735.969 | 735.984 | .016 | *−+ |
| TN | 6654 | 1 | 1 | 736.126 | 736.140 | .015 | *−+ |
| CPT | 6655 | 1 | 1 | 736.325 | 736.339 | .015 | −−> \| |
| TN | 6656 | 1 | 1 | 736.327 | 736.342 | .016 | *−+ |
| CPT | 6657 | 1 | 1 | 736.345 | 736.359 | .015 | −−> \| |
| TP | 6658 | 1 | 1 | 736.903 | 736.917 | .015 | *−+ |
| TN | 6659 | 1 | 1 | 737.233 | 737.247 | .015 | *−+ |
| TP | 6660 | 1 | 1 | 737.271 | 737.285 | .015 | *−+ |
| TN | 6661 | 1 | 1 | 737.331 | 737.346 | .016 | *−+ |
| GG | 6662 | 1 | 1 | 737.643 | 737.972 | .330 | Group0495 \| |
| TN | 6663 | 1 | 1 | 737.752 | 737.766 | .015 | *−+ |
| CNT | 6664 | 1 | 1 | 737.752 | 737.782 | .031 | −−> \| |
| TP | 6665 | 1 | 1 | 737.761 | 737.775 | .015 | *−+ |
| TN | 6666 | 1 | 1 | 738.049 | 738.063 | .015 | *−+ |
| GG | 6667 | 1 | 1 | 738.107 | 739.835 | 1.728 | Group0496 \| |
| TN | 6668 | 1 | 1 | 738.122 | 738.136 | .015 | *−+ |
| TP | 6669 | 1 | 1 | 738.122 | 738.136 | .015 | *−+ |
| TP | 6670 | 1 | 1 | 738.425 | 738.439 | .015 | *−+ |
| TP | 6671 | 1 | 1 | 738.503 | 738.517 | .015 | *−+ |
| CNT | 6672 | 1 | 1 | 738.517 | 738.562 | .046 | −−> \| |
| TN | 6673 | 1 | 1 | 738.544 | 738.559 | .016 | *−+ |
| TN | 6674 | 1 | 1 | 738.862 | 738.878 | .017 | *−+ |
| TP | 6675 | 1 | 1 | 738.862 | 738.878 | .017 | *−+ |
| CNT | 6676 | 1 | 1 | 738.975 | 738.989 | .015 | −−> \| |
| CNT | 6677 | 1 | 1 | 738.992 | 739.007 | .016 | −−> \| |
| TN | 6678 | 1 | 1 | 739.107 | 739.122 | .016 | *−+ |
| GN | 6679 | 1 | 1 | 739.248 | 739.835 | .588 | cheC1 \| |
| TP | 6680 | 1 | 1 | 739.435 | 739.449 | .015 | *−+ |
| TN | 6681 | 1 | 1 | 739.685 | 739.707 | .023 | *−+ |
| TN | 6682 | 1 | 1 | 739.761 | 739.775 | .015 | *−+ |
| GG | 6683 | 1 | 1 | 739.838 | 741.844 | 2.007 | Group0497 \| |
| TN | 6684 | 1 | 1 | 739.849 | 739.863 | .015 | *−+ |
| TP | 6685 | 1 | 1 | 740.345 | 740.360 | .016 | *−+ |
| TN | 6686 | 1 | 1 | 740.362 | 740.376 | .015 | *−+ |
| TP | 6687 | 1 | 1 | 740.611 | 740.625 | .015 | *−+ |
| TN | 6688 | 1 | 1 | 741.100 | 741.116 | .017 | *−+ |
| TP | 6689 | 1 | 1 | 741.101 | 741.132 | .032 | *−+ |
| TN | 6690 | 1 | 1 | 741.218 | 741.232 | .015 | *−+ |
| CNT | 6691 | 1 | 1 | 741.352 | 741.368 | .017 | −−> \| |
| TP | 6692 | 1 | 1 | 741.354 | 741.376 | .023 | *−+ |
| TN | 6693 | 1 | 1 | 741.354 | 741.380 | .027 | *−+ |
| CNT | 6694 | 1 | 1 | 741.374 | 741.388 | .015 | −−> \| |
| TN | 6695 | 1 | 1 | 741.401 | 741.417 | .017 | *−+ |
| CNT | 6696 | 1 | 1 | 741.519 | 741.533 | .015 | −−> \| |
| CNT | 6697 | 1 | 1 | 741.538 | 741.552 | .015 | −−> \| |
| TN | 6698 | 1 | 1 | 741.538 | 741.552 | .015 | *−+ |
| TN | 6699 | 1 | 1 | 741.561 | 741.575 | .015 | *−+ |
| GG | 6700 | 1 | 1 | 741.846 | 743.248 | 1.403 | Group0498 \| |
| TN | 6701 | 1 | 1 | 741.924 | 741.942 | .019 | *−+ |
| TN | 6702 | 1 | 1 | 742.194 | 742.208 | .015 | *−+ |
| TP | 6703 | 1 | 1 | 742.618 | 742.632 | .015 | *−+ |
| TN | 6704 | 1 | 1 | 742.943 | 742.957 | .015 | *−+ |
| GG | 6705 | 1 | 1 | 743.295 | 743.831 | .537 | Group0499 \| |
| TP | 6706 | 1 | 1 | 743.730 | 743.748 | .019 | *−+ |
| GG | 6707 | 1 | 1 | 743.896 | 744.489 | .594 | Group0500 \| |
| TN | 6708 | 1 | 1 | 743.916 | 743.930 | .015 | *−+ |
| GG | 6709 | 1 | 1 | 744.606 | 745.511 | .906 | Group0501 \| |
| TN | 6710 | 1 | 1 | 744.950 | 744.964 | .015 | *−+ |
| TN | 6711 | 1 | 1 | 745.047 | 745.061 | .015 | *−+ |
| GG | 6712 | 1 | 1 | 745.648 | 746.763 | 1.116 | Group0502 \| |
| CPT | 6713 | 1 | 1 | 745.865 | 745.879 | .015 | −−> \| |
| TN | 6714 | 1 | 1 | 745.865 | 745.879 | .015 | *−+ |
| CPT | 6715 | 1 | 1 | 745.888 | 745.902 | .015 | −−> \| |
| CPT | 6716 | 1 | 1 | 746.140 | 746.155 | .016 | −−> \| |
| TN | 6717 | 1 | 1 | 746.153 | 746.175 | .023 | *−+ |
| TP | 6718 | 1 | 1 | 746.157 | 746.175 | .019 | *−+ |
| CPT | 6719 | 1 | 1 | 746.162 | 746.176 | .015 | −−> \| |
| TN | 6720 | 1 | 1 | 746.229 | 746.243 | .015 | *−+ |
| CNT | 6721 | 1 | 1 | 746.229 | 746.259 | .031 | −−> \| |
| TN | 6722 | 1 | 1 | 746.271 | 746.285 | .015 | *−+ |
| TP | 6723 | 1 | 1 | 746.356 | 746.380 | .025 | *−+ |
| TN | 6724 | 1 | 1 | 746.361 | 746.380 | .020 | *−+ |
| TN | 6725 | 1 | 1 | 746.386 | 746.400 | .015 | *−+ |
| CPT | 6726 | 1 | 1 | 746.453 | 746.467 | .015 | −−> \| |
| TN | 6727 | 1 | 1 | 746.453 | 746.472 | .020 | *−+ |
| TP | 6728 | 1 | 1 | 746.457 | 746.475 | .019 | *−+ |
| CPT | 6729 | 1 | 1 | 746.469 | 746.483 | .015 | −−> \| |
| TP | 6730 | 1 | 1 | 746.528 | 746.545 | .018 | *−+ |
| TN | 6731 | 1 | 1 | 746.532 | 746.556 | .025 | *−+ |
| GG | 6732 | 1 | 1 | 746.843 | 748.851 | 2.008 | Group0503 \| |
| CNT | 6733 | 1 | 1 | 746.905 | 746.919 | .015 | −−> \| |
| CNT | 6734 | 1 | 1 | 746.927 | 746.941 | .015 | −−> \| |
| GN | 6735 | 1 | 1 | 747.151 | 748.605 | 1.455 | VNG0983C \| |
| TN | 6736 | 1 | 1 | 747.260 | 747.274 | .015 | *−+ |
| TP | 6737 | 1 | 1 | 747.475 | 747.489 | .015 | *−+ |
| TN | 6738 | 1 | 1 | 747.547 | 747.571 | .025 | *−+ |
| TN | 6739 | 1 | 1 | 747.934 | 747.948 | .015 | *−+ |
| TP | 6740 | 1 | 1 | 747.936 | 747.950 | .015 | *−+ |
| TN | 6741 | 1 | 1 | 748.130 | 748.144 | .015 | *−+ |
| TP | 6742 | 1 | 1 | 749.157 | 749.172 | .016 | *−+ |
| TN | 6743 | 1 | 1 | 749.375 | 749.389 | .015 | *−+ |
| GG | 6744 | 1 | 1 | 749.392 | 759.277 | 9.885 | Group0504 \| |
| CNT | 6745 | 1 | 1 | 759.509 | 759.552 | .044 | −−> \| |
| TP | 6746 | 1 | 1 | 759.509 | 760.014 | .506 | *−+ |
| TN | 6747 | 1 | 1 | 759.528 | 760.031 | .504 | *−+ |
| GG | 6748 | 1 | 1 | 759.667 | 762.487 | 2.820 | Group0505 \| |
| GP | 6749 | 1 | 1 | 760.682 | 762.487 | 1.806 | boa4 \| |
| CPT | 6750 | 1 | 1 | 760.774 | 760.789 | .016 | −−> \| |
| CPT | 6751 | 1 | 1 | 760.798 | 760.814 | .017 | −−> \| |
| TN | 6752 | 1 | 1 | 760.875 | 760.889 | .015 | *−+ |
| CPT | 6753 | 1 | 1 | 760.878 | 760.892 | .015 | −−> \| |
| CPT | 6754 | 1 | 1 | 760.897 | 760.912 | .016 | −−> \| |
| CPT | 6755 | 1 | 1 | 761.496 | 761.510 | .015 | −−> \| |
| CPT | 6756 | 1 | 1 | 761.516 | 761.533 | .018 | −−> \| |
| CPT | 6757 | 1 | 1 | 761.542 | 761.557 | .016 | −−> \| |
| TN | 6758 | 1 | 1 | 761.542 | 761.557 | .016 | *−+ |
| CPT | 6759 | 1 | 1 | 761.576 | 761.597 | .022 | −−> \| |
| CPT | 6760 | 1 | 1 | 761.601 | 761.617 | .017 | −−> \| |
| TN | 6761 | 1 | 1 | 761.754 | 761.768 | .015 | *−+ |
| CPT | 6762 | 1 | 1 | 761.933 | 761.950 | .018 | −−> \| |
| TP | 6763 | 1 | 1 | 761.933 | 761.950 | .018 | *−+ |
| CPT | 6764 | 1 | 1 | 761.953 | 761.968 | .016 | −−> \| |
| TN | 6765 | 1 | 1 | 762.423 | 762.437 | .015 | *−+ |
| GG | 6766 | 1 | 1 | 762.609 | 764.612 | 2.004 | Group0506 \| |
| TN | 6767 | 1 | 1 | 762.874 | 762.900 | .027 | *−+ |
| TN | 6768 | 1 | 1 | 762.946 | 762.960 | .015 | *−+ |
| TN | 6769 | 1 | 1 | 763.108 | 763.122 | .015 | *−+ |
| TN | 6770 | 1 | 1 | 763.273 | 763.304 | .032 | *−+ |
| CPT | 6771 | 1 | 1 | 763.283 | 763.304 | .022 | −−> \| |
| TN | 6772 | 1 | 1 | 763.306 | 763.320 | .015 | *−+ |
| CPT | 6773 | 1 | 1 | 763.306 | 763.321 | .016 | −−> \| |
| CPT | 6774 | 1 | 1 | 763.346 | 763.360 | .015 | −−> \| |
| TN | 6775 | 1 | 1 | 763.351 | 763.365 | .015 | *−+ |
| TP | 6776 | 1 | 1 | 763.351 | 763.365 | .015 | *−+ |
| CPT | 6777 | 1 | 1 | 763.362 | 763.376 | .015 | −−> \| |
| TP | 6778 | 1 | 1 | 763.391 | 763.407 | .017 | *−+ |
| TN | 6779 | 1 | 1 | 763.843 | 763.857 | .015 | *−+ |
| TP | 6780 | 1 | 1 | 763.843 | 763.857 | .015 | *−+ |
| CNT | 6781 | 1 | 1 | 763.965 | 763.986 | .022 | −−> \| |
| CPT | 6782 | 1 | 1 | 763.965 | 763.995 | .031 | −−> \| |
| CNT | 6783 | 1 | 1 | 763.990 | 764.011 | .022 | −−> \| |
| CPT | 6784 | 1 | 1 | 764.000 | 764.015 | .016 | −−> \| |
| CNT | 6785 | 1 | 1 | 764.075 | 764.089 | .015 | −−> \| |
| CNT | 6786 | 1 | 1 | 764.093 | 764.108 | .016 | −−> \| |
| CPT | 6787 | 1 | 1 | 764.156 | 764.171 | .016 | −−> \| |
| CNT | 6788 | 1 | 1 | 764.156 | 764.177 | .022 | OS−> \| |
| TN | 6789 | 1 | 1 | 764.156 | 764.177 | .022 | *−+ |
| CNT | 6790 | 1 | 1 | 764.181 | 764.195 | .015 | −−> \| |
| TP | 6791 | 1 | 1 | 764.181 | 764.201 | .021 | *−+ |
| CPT | 6792 | 1 | 1 | 764.181 | 764.204 | .024 | −−> \| |
| CPT | 6793 | 1 | 1 | 764.213 | 764.227 | .013 | −−> \| |
| TP | 6794 | 1 | 1 | 764.309 | 764.328 | .020 | *−+ |
| TN | 6795 | 1 | 1 | 764.312 | 764.326 | .015 | *−+ |
| TN | 6796 | 1 | 1 | 764.383 | 764.397 | .015 | *−+ |
| TP | 6797 | 1 | 1 | 764.530 | 764.545 | .016 | *−+ |
| TN | 6798 | 1 | 1 | 764.530 | 764.548 | .019 | *−+ |
| GG | 6799 | 1 | 1 | 764.619 | 765.629 | 1.011 | Group0507 \| |
| CPT | 6800 | 1 | 1 | 764.791 | 764.808 | .018 | −−> \| |
| CNT | 6801 | 1 | 1 | 764.794 | 764.813 | .020 | OS−> \| |
| TN | 6802 | 1 | 1 | 764.794 | 764.832 | .039 | *−+ |
| CPT | 6803 | 1 | 1 | 764.810 | 764.826 | .017 | −−> \| |
| CNT | 6804 | 1 | 1 | 764.818 | 764.832 | .015 | −−> \| |
| CPT | 6805 | 1 | 1 | 764.831 | 764.853 | .023 | −−> \| |
| CPT | 6806 | 1 | 1 | 764.856 | 764.870 | .015 | −−> \| |
| CNT | 6807 | 1 | 1 | 764.984 | 764.998 | .015 | OS−> \| |
| TN | 6808 | 1 | 1 | 764.984 | 765.014 | .031 | *−+ |
| CNT | 6809 | 1 | 1 | 765.000 | 765.014 | .015 | −−> \| |
| TN | 6810 | 1 | 1 | 765.075 | 765.089 | .015 | *−+ |
| CNT | 6811 | 1 | 1 | 765.431 | 765.446 | .016 | −−> \| |
| CNT | 6812 | 1 | 1 | 765.456 | 765.473 | .018 | −−> \| |
| CNT | 6813 | 1 | 1 | 765.479 | 765.494 | .016 | −−> \| |
| TN | 6814 | 1 | 1 | 765.508 | 765.523 | .016 | *−+ |
| TP | 6815 | 1 | 1 | 765.573 | 765.587 | .015 | *−+ |
| GG | 6816 | 1 | 1 | 765.775 | 766.044 | .270 | Group0508 \| |
| TN | 6817 | 1 | 1 | 765.822 | 765.837 | .016 | *−+ |
| TP | 6818 | 1 | 1 | 766.162 | 766.176 | .015 | *−+ |
| TN | 6819 | 1 | 1 | 766.432 | 766.447 | .016 | *−+ |
| GG | 6820 | 1 | 1 | 766.603 | 766.812 | .210 | Group0509 \| |
| CNT | 6821 | 1 | 1 | 766.635 | 766.653 | .019 | OS−> \| |
| TN | 6822 | 1 | 1 | 766.635 | 766.653 | .019 | *−+ |
| CNT | 6823 | 1 | 1 | 766.655 | 766.675 | .021 | −−> \| |
| TN | 6824 | 1 | 1 | 766.863 | 766.878 | .016 | *−+ |
| GG | 6825 | 1 | 1 | 766.955 | 768.538 | 1.584 | Group0510 \| |
| TN | 6826 | 1 | 1 | 767.682 | 767.700 | .019 | *−+ |
| TP | 6827 | 1 | 1 | 767.831 | 767.845 | .015 | *−+ |
| TN | 6828 | 1 | 1 | 768.028 | 768.048 | .021 | *−+ |
| TP | 6829 | 1 | 1 | 768.033 | 768.049 | .017 | *−+ |
| TN | 6830 | 1 | 1 | 768.377 | 768.391 | .015 | *−+ |
| GG | 6831 | 1 | 1 | 768.965 | 769.882 | .918 | Group0511 \| |
| TN | 6832 | 1 | 1 | 769.502 | 769.516 | .015 | *−+ |
| TN | 6833 | 1 | 1 | 769.541 | 769.555 | .015 | *−+ |
| TP | 6834 | 1 | 1 | 769.559 | 769.573 | .015 | *−+ |
| GG | 6833 | 1 | 1 | 769.965 | 770.189 | .225 | Group0512 \| |
| TN | 6836 | 1 | 1 | 770.120 | 770.134 | .015 | *−+ |
| GG | 6837 | 1 | 1 | 770.194 | 770.406 | .213 | Group0513 \| |
| CPT | 6838 | 1 | 1 | 770.226 | 770.240 | .015 | −−> \| |
| CPT | 6839 | 1 | 1 | 770.251 | 770.265 | .015 | −−> \| |
| TP | 6840 | 1 | 1 | 770.251 | 770.268 | .018 | *−+ |
| CPT | 6841 | 1 | 1 | 770.347 | 770.363 | .017 | −−> \| |
| CPT | 6842 | 1 | 1 | 770.365 | 770.379 | .015 | −−> \| |
| GG | 6843 | 1 | 1 | 770.516 | 771.546 | 1.030 | Group0514 \| |
| TN | 6844 | 1 | 1 | 771.564 | 772.071 | .508 | *−+ |
| CNT | 6845 | 1 | 1 | 771.564 | 772.086 | .523 | −−> \| |
| TP | 6846 | 1 | 1 | 771.583 | 772.084 | .502 | *−+ |
| GG | 6847 | 1 | 1 | 771.722 | 771.925 | .204 | Group0515 \| |
| TN | 6848 | 1 | 1 | 772.963 | 772.977 | .015 | *−+ |
| TP | 6849 | 1 | 1 | 773.358 | 773.387 | .030 | *−+ |
| GG | 6850 | 1 | 1 | 773.360 | 773.950 | .591 | Group0516 \| |
| CPT | 6851 | 1 | 1 | 773.399 | 773.483 | .085 | OS−> \| |
| TP | 6852 | 1 | 1 | 773.399 | 773.483 | .085 | *−* |

The “Type” descriptor of the T1 (Id number 6644a) is “TP” showing that is T1 target is on the negative strand of the double-stranded genomic DNA. Because the T1 and the T2 targets have to be on the same strand, the T2 target (Id number 6852) also has the type descriptor “TP”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is changed to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Group0493 (Id number 6646) has the type descriptor “GG”. The same is true for the group headers through to Group0516 (Id number 6847).

| Gene_Name | COG_Id | | Chromosome | Direction | Start | Stop | Length |
|---|---|---|---|---|---|---|---|
| Group0493 | | | | | | | |
| VNG0964C | COG2469 | S | 1 | positive | 735.009 | 735.881 | .873 |
| Group0494 | | | | | | | |
| VNG0965C | COG2469 | S | 1 | negative | 735.910 | 736.773 | .864 |
| cheR | COG1352 | N | 1 | negative | 736.840 | 737.544 | .705 |
| Group0495 | | | | | | | |
| cheD | COG1871 | N | 1 | negative | 737.643 | 737.972 | .330 |
| Group0496 | | | | | | | |
| VNG0969H | COG1776 | N | 1 | negative | 738.107 | 739.246 | 1.140 |
| cheC1 | COG1776 | N | 1 | negative | 739.248 | 739.835 | .588 |
| Group0497 | | | | | | | |
| cheA | COG0643 | N | 1 | negative | 739.838 | 741.844 | 2.007 |
| Group0498 | | | | | | | |
| cheB | COG2201 | N | 1 | negative | 741.846 | 743.248 | 1.403 |
| Group0499 | | | | | | | |
| cheW1 | COG0835 | N | 1 | negative | 743.295 | 743.831 | .537 |
| Group0500 | | | | | | | |
| VNG0978H | | | 1 | negative | 743.896 | 744.489 | .594 |
| Group0501 | | | | | | | |
| VNG0979H | | | 1 | positive | 744.606 | 745.511 | .906 |
| Group0502 | | | | | | | |
| VNG0981C | COG0436 | E | 1 | positive | 745.648 | 746.763 | 1.116 |
| Group0503 | | | | | | | |
| VNG0982C | COG1873 | S | 1 | positive | 746.843 | 747.133 | .291 |
| VNG0983C | COG0618 | R | 1 | negative | 747.151 | 748.605 | 1.455 |
| tRNA-Lys | | | 1 | positive | 748.778 | 748.851 | .074 |
| Group0504 | | | | | | | |
| VNG0985H | | | 1 | positive | 749.392 | 751.398 | 2.007 |
| VNG0986H | | | 1 | positive | 752.276 | 752.803 | .528 |
| VNG0987H | | | 1 | positive | 753.131 | 753.547 | .417 |
| VNG0988H | | | 1 | positive | 754.149 | 754.391 | .243 |
| VNG0989C | COG0582 | L | 1 | negative | 754.459 | 755.481 | 1.023 |
| VNG0990H | | | 1 | positive | 756.010 | 756.183 | .174 |
| VNG0991H | | | 1 | negative | 756.675 | 757.016 | .342 |
| VNG0992H | | | 1 | negative | 757.409 | 758.119 | .711 |
| VNG0993H | | | 1 | positive | 758.465 | 759.277 | .813 |
| Group0505 | | | | | | | |
| VNG0994H | | | 1 | negative | 759.667 | 759.870 | .204 |
| VNG0995H | | | 1 | positive | 760.365 | 760.553 | .189 |
| boa4 | COG3413 | R | 1 | positive | 760.682 | 762.487 | 1.806 |
| Group0506 | | | | | | | |
| acs2 | COG0365 | I | 1 | positive | 762.609 | 764.612 | 2.004 |
| Group0507 | | | | | | | |
| yajO2 | COG0667 | C | 1 | negative | 764.619 | 765.629 | 1.011 |
| Group0508 | | | | | | | |
| VNG0999H | | | 1 | negative | 765.775 | 766.044 | .270 |
| Group0509 | | | | | | | |
| VNG1000H | COG3205 | S | 1 | positive | 766.603 | 766.812 | .210 |
| Group0510 | | | | | | | |
| guaB | COG0516 | F | 1 | negative | 766.955 | 768.538 | 1.584 |
| Group0511 | | | | | | | |
| VNG1002H | | | 1 | positive | 768.965 | 769.882 | .918 |
| Group0512 | | | | | | | |
| VNG1003H | | | 1 | positive | 769.965 | 770.189 | .225 |
| Group0513 | | | | | | | |
| VNG1005H | | | 1 | negative | 770.194 | 770.406 | .213 |
| Group0514 | | | | | | | |
| tRNA-Trp | | | 1 | positive | 770.516 | 770.691 | .176 |
| VNG1006H | | | 1 | positive | 770.827 | 771.546 | .720 |
| Group0515 | | | | | | | |
| VNG1007H | | | 1 | negative | 771.722 | 771.925 | .204 |
| Group0516 | | | | | | | |
| flaAla | COG1681 | N | 1 | positive | 773.360 | 773.950 | .591 |

All of the data for the transient connectron 7341 are pulled together in the following table that is the “terse” description of the connectron.

| Connectron Relationships - | Global_Id | Type |
|---|---|---|
| | 7341 | transient |

| | | Chro- | | | | |
|---|---|---|---|---|---|---|
| Control | | mo- | C1/ | | | |
| Sequences - | Direction | some | C2_Id | Start | Stop | Length |
| | positive | 1 | 6612 | 732.401 | 732.534 | .134 |

| Trigger Gene - | Name | COG_Id | Start | Stop | Length |
|---|---|---|---|---|---|
| | flaBl | COG1681 | 731.799 | 732.380 | .582 |

| | | Chro- | | | | |
|---|---|---|---|---|---|---|
| Target | | mo- | | | | |
| Sequences - | Direction | some | | Start | Stop | Length |
| | | | T1_Id | | | |
| | positive | 1 | 6644a | 734.750 | 734.942 | .193 |
| | | | T2_Id | | | |
| | | | 6852 | 773.399 | 773.483 | .085 |

Controlled Genes

| Local_Id | Chromosome | Group | Name | COG_Id | Direction | Start | Stop | Length |
|---|---|---|---|---|---|---|---|---|
| 1 | 1 | Group0493 | VNG0964C | COG2469 | positive | 735.009 | 735.881 | .873 |
| 2 | 1 | Group0494 | VNG0965C | COG2469 | positive | 735.910 | 737.544 | 1.634 |
| 3 | 1 | Group0494 | cheR | COG1352 | negative | 737.544 | 736.840 | .705 |
| 4 | 1 | Group0495 | cheD | COG1871 | positive | 737.643 | 737.972 | .330 |
| 5 | 1 | Group0496 | VNG0969H | COG1776 | positive | 738.107 | 739.835 | 1.728 |
| 6 | 1 | Group0496 | cheC1 | COG1776 | negative | 739.835 | 739.248 | .588 |
| 7 | 1 | Group0497 | cheA | COG0643 | positive | 739.838 | 741.844 | 2.007 |
| 8 | 1 | Group0498 | cheB | COG2201 | positive | 741.846 | 743.248 | 1.403 |
| 9 | 1 | Group0499 | cheW1 | COG0835 | positive | 743.295 | 743.831 | .537 |
| 10 | 1 | Group0500 | VNG0978H | | positive | 743.896 | 744.489 | .594 |
| 11 | 1 | Group0501 | VNG0979H | | positive | 744.606 | 745.511 | .906 |
| 12 | 1 | Group0502 | VNG0981C | COG0436 | positive | 745.648 | 746.763 | 1.116 |
| 13 | 1 | Group0503 | VNG0982C | COG1873 | positive | 746.843 | 748.851 | 2.008 |
| 14 | 1 | Group0503 | VNG0983C | COG0618 | negative | 748.605 | 747.151 | 1.455 |
| 15 | 1 | Group0504 | VNG0985H | | positive | 749.392 | 759.277 | 9.885 |
| 16 | 1 | Group0504 | VNG0986H | | positive | 752.276 | 752.803 | .528 |
| 17 | 1 | Group0504 | VNG0987H | | positive | 753.131 | 753.547 | .417 |
| 18 | 1 | Group0504 | VNG0988H | | positive | 754.149 | 754.391 | .243 |
| 19 | 1 | Group0504 | VNG0989C | COG0582 | negative | 755.481 | 754.459 | 1.023 |
| 20 | 1 | Group0504 | VNG0990H | | positive | 756.010 | 756.183 | .174 |
| 21 | 1 | Group0504 | VNG0991H | | negative | 757.016 | 756.675 | .342 |
| 22 | 1 | Group0504 | VNG0992H | | negative | 758.119 | 757.409 | .711 |
| 23 | 1 | Group0504 | VNG0993H | | positive | 758.465 | 759.277 | .813 |
| 24 | 1 | Group0505 | VNG0994H | | positive | 759.667 | 762.487 | 2.820 |
| 25 | 1 | Group0505 | VNG0995H | | positive | 760.365 | 760.553 | .189 |
| 26 | 1 | Group0505 | boa4 | COG3413 | positive | 760.682 | 762.487 | 1.806 |
| 27 | 1 | Group0506 | acs2 | COG0365 | positive | 762.609 | 764.612 | 2.004 |
| 28 | 1 | Group0507 | yajO2 | COG0667 | positive | 764.619 | 765.629 | 1.011 |
| 29 | 1 | Group0508 | VNG0999H | | positive | 765.775 | 766.044 | .270 |
| 30 | 1 | Group0509 | VNG1000H | COG3205 | positive | 766.603 | 766.812 | .210 |
| 31 | 1 | Group0510 | guaB | COG0516 | positive | 766.955 | 768.538 | 1.584 |
| 32 | 1 | Group0511 | VNG1002H | | positive | 768.965 | 769.882 | .918 |
| 33 | 1 | Group0512 | VNG1003H | | positive | 769.965 | 770.189 | .225 |
| 34 | 1 | Group0513 | VNG1005H | | positive | 770.194 | 770.406 | .213 |
| 35 | 1 | Group0514 | tRNA-Trp | | positive | 770.516 | 771.546 | 1.030 |
| 36 | 1 | Group0514 | VNG1006H | | positive | 770.827 | 771.546 | .720 |
| 37 | 1 | Group0515 | VNG1007H | | positive | 771.722 | 771.925 | .204 |
| 38 | 1 | Group0516 | flaAla | COG1681 | positive | 773.360 | 773.950 | .591 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 6644 | positive | 734.735 | 734.749 | .015 |
| 2 | 1 | 6647 | negative | 735.378 | 735.364 | .015 |
| 3 | 1 | 6649 | negative | 735.403 | 735.389 | .015 |
| 4 | 1 | 6650 | negative | 735.767 | 735.753 | .015 |
| 5 | 1 | 6651 | negative | 735.789 | 735.775 | .015 |
| 6 | 1 | 6655 | positive | 736.325 | 736.339 | .015 |
| 7 | 1 | 6657 | positive | 736.345 | 736.359 | .015 |
| 8 | 1 | 6664 | negative | 737.782 | 737.752 | .031 |
| 9 | 1 | 6672 | negative | 738.562 | 738.517 | .046 |
| 10 | 1 | 6676 | negative | 738.989 | 738.975 | .015 |
| 11 | 1 | 6677 | negative | 739.007 | 738.992 | .016 |
| 12 | 1 | 6691 | negative | 741.368 | 741.352 | .017 |
| 13 | 1 | 6694 | negative | 741.388 | 741.374 | .015 |
| 14 | 1 | 6696 | negative | 741.533 | 741.519 | .015 |
| 15 | 1 | 6697 | negative | 741.552 | 741.538 | .015 |
| 16 | 1 | 6713 | positive | 745.865 | 745.879 | .015 |
| 17 | 1 | 6715 | positive | 745.888 | 745.902 | .015 |
| 18 | 1 | 6716 | positive | 746.140 | 746.155 | .016 |
| 19 | 1 | 6719 | positive | 746.162 | 746.176 | .015 |
| 20 | 1 | 6721 | negative | 746.259 | 746.229 | .031 |
| 21 | 1 | 6726 | positive | 746.453 | 746.467 | .015 |
| 22 | 1 | 6729 | positive | 746.469 | 746.483 | .015 |
| 23 | 1 | 6733 | negative | 746.919 | 746.905 | .015 |
| 24 | 1 | 6734 | negative | 746.941 | 746.927 | .015 |
| 25 | 1 | 6745 | negative | 759.552 | 759.509 | .044 |
| 26 | 1 | 6750 | positive | 760.774 | 760.789 | .016 |
| 27 | 1 | 6751 | positive | 760.798 | 760.814 | .017 |
| 28 | 1 | 6753 | positive | 760.878 | 760.892 | .015 |
| 29 | 1 | 6754 | positive | 760.897 | 760.912 | .016 |
| 30 | 1 | 6755 | positive | 761.496 | 761.510 | .015 |
| 31 | 1 | 6756 | positive | 761.516 | 761.533 | .018 |
| 32 | 1 | 6757 | positive | 761.542 | 761.557 | .016 |
| 33 | 1 | 6759 | positive | 761.576 | 761.597 | .022 |
| 34 | 1 | 6760 | positive | 761.601 | 761.617 | .017 |
| 35 | 1 | 6762 | positive | 761.933 | 761.950 | .018 |
| 36 | 1 | 6764 | positive | 761.953 | 761.968 | .016 |
| 37 | 1 | 6771 | positive | 763.283 | 763.304 | .022 |
| 38 | 1 | 6773 | positive | 763.306 | 763.321 | .016 |
| 39 | 1 | 6774 | positive | 763.346 | 763.360 | .015 |
| 40 | 1 | 6777 | positive | 763.362 | 763.376 | .015 |
| 41 | 1 | 6781 | negative | 763.986 | 763.965 | .022 |
| 42 | 1 | 6782 | positive | 763.965 | 763.995 | .031 |
| 43 | 1 | 6783 | negative | 764.011 | 763.990 | .022 |
| 44 | 1 | 6784 | positive | 764.000 | 764.015 | .016 |
| 45 | 1 | 6785 | negative | 764.089 | 764.075 | .015 |
| 46 | 1 | 6786 | negative | 764.108 | 764.093 | .016 |
| 47 | 1 | 6787 | positive | 764.156 | 764.171 | .016 |
| 48 | 1 | 6788 | negative | 764.177 | 764.156 | .022 |
| 49 | 1 | 6790 | negative | 764.195 | 764.181 | .015 |
| 50 | 1 | 6792 | positive | 764.181 | 764.204 | .024 |
| 51 | 1 | 6793 | positive | 764.213 | 764.227 | .015 |
| 52 | 1 | 6800 | positive | 764.791 | 764.808 | .018 |
| 53 | 1 | 6801 | negative | 764.813 | 764.794 | .020 |
| 54 | 1 | 6803 | positive | 764.810 | 764.826 | .017 |
| 55 | 1 | 6804 | negative | 764.832 | 764.818 | .015 |
| 56 | 1 | 6805 | positive | 764.831 | 764.853 | .023 |
| 57 | 1 | 6806 | positive | 764.856 | 764.870 | .015 |
| 58 | 1 | 6807 | negative | 764.998 | 764.984 | .015 |
| 59 | 1 | 6809 | negative | 765.014 | 765.000 | .015 |
| 60 | 1 | 6811 | negative | 765.446 | 765.431 | .016 |
| 61 | 1 | 6812 | negative | 765.473 | 765.456 | .018 |
| 62 | 1 | 6813 | negative | 765.494 | 765.479 | .016 |
| 63 | 1 | 6821 | negative | 766.653 | 766.635 | .019 |
| 64 | 1 | 6823 | negative | 766.675 | 766.655 | .021 |
| 65 | 1 | 6838 | positive | 770.226 | 770.240 | .015 |
| 66 | 1 | 6839 | positive | 770.251 | 770.265 | .015 |
| 67 | 1 | 6841 | positive | 770.347 | 770.363 | .017 |
| 68 | 1 | 6842 | positive | 770.365 | 770.379 | .015 |
| 69 | 1 | 6845 | negative | 772.086 | 771.564 | .523 |
| 70 | 1 | 6851 | positive | 773.399 | 773.483 | .085 |

The verbose description of the transient connectron 7341 is:

In the Halobacterium sp. NRC-1 complete genome the transient connectron number 7341 is generated by the control sequence (C1/C2) whose identifier number is 6612. This control sequence is on the positive strand of the genomic DNA of chromosome 1. The genomic start and stop positions of this control sequence are 732.401 KB and 732.534 KB with a length of 0.134 KB. Expression of the RNA for this connectron is triggered by the promotion of the gene whose name is flaB1 and whose COG (Cluster of Orthologous Genes) identifier is COG1681. The genomic start and stop positions of this gene are 731.799 KB and 732.380 KB and with a length of 0.582 KB. This connectron causes stabilization of a loop of DNA. The target sequences (T1-T2) are on the positive strand of the genomic DNA on chromosome 1. The identifier number of the initiating target sequence (T1) is 6644a. The genomic start and stop positions of this initiating target sequence are 734.750 KB and 734.942 KB with a length of 0.193 KB. The identifier number of the terminating target sequence (T2) is 6852. The genomic start and stop positions of this terminating target sequence are 773.399 KB and 773.483 KB with a length of 0.085 KB.

This connectron controls the modulation of the expression of the following gene(s): (1) The gene that has the name VNG0964C and with a COG identifier of COG2469. The genomic start and stop positions of this gene are 735.009 KB and 735.881 KB with a length of 0.873 KB. (2) The gene that has the name VNG0965C and with a COG identifier of COG2469. The genomic start and stop positions of this gene are 735.910 KB and 737.544 KB with a length of 1.634 KB. (3) The gene that has the name cheR and with a COG identifier of COG1352. The genomic start and stop positions of this gene are 737.544 KB and 736.840 KB with a length of 0.705 KB. (4) The gene that has the name cheD and with a COG identifier of COG1871. The genomic start and stop positions of this gene are 737.643 KB and 737.972 KB with a length of 0.330 KB. (5) The gene that has the name VNG0969H and with a COG identifier of COG1776. The genomic start and stop positions of this gene are 738.107 KB and 739.835 KB with a length of 1.728 KB. (6) The gene that has the name cheC1 and with a COG identifier of COG1776. The genomic start and stop positions of this gene are 739.835 KB and 739.248 KB with a length of 0.588 KB. (7) The gene that has the name cheA and with a COG identifier of COG0643. The genomic start and stop positions of this gene are 739.838 KB and 741.844 KB with a length of 2.007 KB. (8) The gene that has the name cheB and with a COG identifier of COG2201. The genomic start and stop positions of this gene are 741.846 KB and 743.248 KB with a length of 1.403 KB. (9) The gene that has the name cheW1 and with a COG identifier of COG0835. The genomic start and stop positions of this gene are 743.295 KB and 743.831 KB with a length of 0.537 KB. (10) The gene that has the name VNG0978H. The genomic start and stop positions of this gene are 743.896 KB and 744.489 KB with a length of 0.594 KB. (11) The gene that has the name VNG0979H. The genomic start and stop positions of this gene are 744.606 KB and 745.511 KB with a length of 0.906 KB. (12) The gene that has the name VNG0981C and with a COG identifier of COG0436. The genomic start and stop positions of this gene are 745.648 KB and 746.763 KB with a length of 1.116 KB. (13) The gene that has the name VNG0982C and with a COG identifier of COG1873. The genomic start and stop positions of this gene are 746.843 KB and 748.851 KB with a length of 2.008 KB. (14) The gene that has the name VNG0983C and with a COG identifier of COG0618. The genomic start and stop positions of this gene are 748.605 KB and 747.151 KB with a length of 1.455 KB. (15) The gene that has the name VNG0985H. The genomic start and stop positions of this gene are 749.392 KB and 759.277 KB with a length of 9.885 KB. (16) The gene that has the name VNG0986H. The genomic start and stop positions of this gene are 752.276 KB and 752.803 KB with a length of 0.528 KB. (17) The gene that has the name VNG0987H. The genomic start and stop positions of this gene are 753.131 KB and 753.547 KB with a length of 0.417 KB. (18) The gene that has the name VNG0988H. The genomic start and stop positions of this gene are 754.149 KB and 754.391 KB with a length of 0.243 KB. (19) The gene that has the name VNG0989C and with a COG identifier of COG0582. The genomic start and stop positions of this gene are 755.481 KB and 754.459 KB with a length of 1.023 KB. (20) The gene that has the name VNG0990H. The genomic start and stop positions of this gene are 756.010 KB and 756.183 KB with a length of 0.174 KB. (21) The gene that has the name VNG0991H. The genomic start and stop positions of this gene are 757.016 KB and 756.675 KB with a length of 0.342 KB. (22) The gene that has the name VNG0992H. The genomic start and stop positions of this gene are 758.119 KB and 757.409 KB with a length of 0.711 KB. (23) The gene that has the name VNG0993H. The genomic start and stop positions of this gene are 758.465 KB and 759.277 KB with a length of 0.813 KB. (24) The gene that has the name VNG0994H. The genomic start and stop positions of this gene are 759.667 KB and 762.487 KB with a length of 2.820 KB. (25) The gene that has the name VNG0995H. The genomic start and stop positions of this gene are 760.365 KB and 760.553 KB with a length of 0.189 KB. (26) The gene that has the name boa4 and with a COG identifier of COG3413. The genomic start and stop positions of this gene are 760.682 KB and 762.487 KB with a length of 1.806 KB. (27) The gene that has the name acs2 and with a COG identifier of COG0365. The genomic start and stop positions of this gene are 762.609 KB and 764.612 KB with a length of 2.004 KB. (28) The gene that has the name yajO2 and with a COG identifier of COG0667. The genomic start and stop positions of this gene are 764.619 KB and 765.629 KB with a length of 1.011 KB. (29) The gene that has the name VNG0999H. The genomic start and stop positions of this gene are 765.775 KB and 766.044 KB with a length of 0.270 KB. (30) The gene that has the name VNG1000H and with a COG identifier of COG3205. The genomic start and stop positions of this gene are 766.603 KB and 766.812 KB with a length of 0.210 KB. (31) The gene that has the name guaB and with a COG identifier of COG0516. The genomic start and stop positions of this gene are 766.955 KB and 768.538 KB with a length of 1.584 KB. (32) The gene that has the name VNG1002H. The genomic start and stop positions of this gene are 768.965 KB and 769.882 KB with a length of 0.918 KB. (33) The gene that has the name VNG1003H. The genomic start and stop positions of this gene are 769.965 KB and 770.189 KB with a length of 0.225 KB. (34) The gene that has the name VNG1005H. The genomic start and stop positions of this gene are 770.194 KB and 770.406 KB with a length of 0.213 KB. (35) The gene that has the name tRNA-Trp. The genomic start and stop positions of this gene are 770.516 KB and 771.546 KB with a length of 1.030 KB. (36) The gene that has the name VNG1006H. The genomic start and stop positions of this gene are 770.827 KB and 771.546 KB with a length of 0.720 KB. (37) The gene that has the name VNG1007H. The genomic start and stop positions of this gene are 771.722 KB and 771.925 KB with a length of 0.204 KB. (38) The gene that has the name flaA1a and with a COG identifier of COG1681. The genomic start and stop positions of this gene are 773.360 KB and 773.950 KB with a length of 0.591 KB.

This connectron controls the turning off of the expression of the following connectron(s): (1) The connectron that has the identifier 6644 is on chromosome 1 in the positive direction. The genomic start and stop positions of this connectron are 734.735 KB and 734.749 KB with a length of 0.015 KB. (2) The connectron that has the identifier 6647 is on chromosome 1 in the negative direction. The genomic start and stop positions of this connectron are 735.378 KB and 735.364 KB with a length of 0.015 KB. (3) The connectron that has the identifier 6649 is on chromosome 1 in the negative direction. The genomic start and stop positions of this connectron are 735.403 KB and 735.389 KB with a length of 0.015 KB. (4) The connectron that has the identifier 6650 is on chromosome 1 in the negative direction. The genomic start and stop positions of this connectron are 735.767 KB and 735.753 KB with a length of 0.015 KB. (5) The connectron that has the identifier 6651 is on chromosome 1 in the negative direction. The genomic start and stop positions of this connectron are 735.789 KB and 735.775 KB with a length of 0.015 KB. (6) The connectron that has the identifier 6655 is on chromosome 1 in the positive direction. The genomic start and stop positions of this connectron are 736.325 KB and 736.339 KB with a length of 0.015 KB. (7) The connectron that has the identifier 6657 is on chromosome 1 in the positive direction. The genomic start and stop positions of this connectron are 736.345 KB and 736.359 KB with a length of 0.015 KB. (8) The connectron that has the identifier 6664 is on chromosome 1 in the negative direction. The genomic start and stop positions of this connectron are 737.782 KB and 737.752 KB with a length of 0.031 KB. (9) The connectron that has the identifier 6672 is on chromosome 1 in the negative direction. The genomic start and stop positions of this connectron are 738.562 KB and 738.517 KB with a length of 0.046 KB. (10) The connectron that has the identifier 6676 is on chromosome 1 in the negative direction. The genomic start and stop positions of this connectron are 738.989 KB and 738.975 KB with a length of 0.015 KB. (11) The connectron that has the identifier 6677 is on chromosome 1 in the negative direction. The genomic start and stop positions of this connectron are 739.007 KB and 738.992 KB with a length of 0.016 KB. (12) The connectron that has the identifier 6691 is on chromosome 1 in the negative direction. The genomic start and stop positions of this connectron are 741.368 KB and 741.352 KB with a length of 0.017 KB. (13) The connectron that has the identifier 6694 is on chromosome 1 in the negative direction. The genomic start and stop positions of this connectron are 741.388 KB and 741.374 KB with a length of 0.015 KB. (14) The connectron that has the identifier 6696 is on chromosome 1 in the negative direction. The genomic start and stop positions of this connectron are 741.533 KB and 741.519 KB with a length of 0.015 KB. (15) The connectron that has the identifier 6697 is on chromosome 1 in the negative direction. The genomic start and stop positions of this connectron are 741.552 KB and 741.538 KB with a length of 0.015 KB. (16) The connectron that has the identifier 6713 is on chromosome 1 in the positive direction. The genomic start and stop positions of this connectron are 745.865 KB and 745.879 KB with a length of 0.015 KB. (17) The connectron that has the identifier 6715 is on chromosome 1 in the positive direction. The genomic start and stop positions of this connectron are 745.888 KB and 745.902 KB with a length of 0.015 KB. (18) The connectron that has the identifier 6716 is on chromosome 1 in the positive direction. The genomic start and stop positions of this connectron are 746.140 KB and 746.155 KB with a length of 0.016 KB. (19) The connectron that has the identifier 6719 is on chromosome 1 in the positive direction. The genomic start and stop positions of this connectron are 746.162 KB and 746.176 KB with a length of 0.015 KB. (20) The connectron that has the identifier 6721 is on chromosome 1 in the negative direction. The genomic start and stop positions of this connectron are 746.259 KB and 746.229 KB with a length of 0.031 KB. (21) The connectron that has the identifier 6726 is on chromosome 1 in the positive direction. The genomic start and stop positions of this connectron are 746.453 KB and 746.467 KB with a length of 0.015 KB. (22) The connectron that has the identifier 6729 is on chromosome 1 in the positive direction. The genomic start and stop positions of this connectron are 746.469 KB and 746.483 KB with a length of 0.015 KB. (23) The connectron that has the identifier 6733 is on chromosome 1 in the negative direction. The genomic start and stop positions of this connectron are 746.919 KB and 746.905 KB with a length of 0.015 KB. (24) The connectron that has the identifier 6734 is on chromosome 1 in the negative direction. The genomic start and stop positions of this connectron are 746.941 KB and 746.927 KB with a length of 0.015 KB. (25) The connectron that has the identifier 6745 is on chromosome 1 in the negative direction. The genomic start and stop positions of this connectron are 759.552 KB and 759.509 KB with a length of 0.044 KB. (26) The connectron that has the identifier 6750 is on chromosome 1 in the positive direction. The genomic start and stop positions of this connectron are 760.774 KB and 760.789 KB with a length of 0.016 KB. (27) The connectron that has the identifier 6751 is on chromosome 1 in the positive direction. The genomic start and stop positions of this connectron are 760.798 KB and 760.814 KB with a length of 0.017 KB. (28) The connectron that has the identifier 6753 is on chromosome 1 in the positive direction. The genomic start and stop positions of this connectron are 760.878 KB and 760.892 KB with a length of 0.015 KB. (29) The connectron that has the identifier 6754 is on chromosome 1 in the positive direction. The genomic start and stop positions of this connectron are 760.897 KB and 760.912 KB with a length of 0.016 KB. (30) The connectron that has the identifier 6755 is on chromosome 1 in the positive direction. The genomic start and stop positions of this connectron are 761.496 KB and 761.510 KB with a length of 0.015 KB. (31) The connectron that has the identifier 6756 is on chromosome 1 in the positive direction. The genomic start and stop positions of this connectron are 761.516 KB and 761.533 KB with a length of 0.018 KB. (32) The connectron that has the identifier 6757 is on chromosome 1 in the positive direction. The genomic start and stop positions of this connectron are 761.542 KB and 761.557 KB with a length of 0.016 KB. (33) The connectron that has the identifier 6759 is on chromosome 1 in the positive direction. The genomic start and stop positions of this connectron are 761.576 KB and 761.597 KB with a length of 0.022 KB. (34) The connectron that has the identifier 6760 is on chromosome 1 in the positive direction. The genomic start and stop positions of this connectron are 761.601 KB and 761.617 KB with a length of 0.017 KB. (35) The connectron that has the identifier 6762 is on chromosome 1 in the positive direction. The genomic start and stop positions of this connectron are 761.933 KB and 761.950 KB with a length of 0.018 KB. (36) The connectron that has the identifier 6764 is on chromosome 1 in the positive direction. The genomic start and stop positions of this connectron are 761.953 KB and 761.968 KB with a length of 0.016 KB. (37) The connectron that has the identifier 6771 is on chromosome 1 in the positive direction. The genomic start and stop positions of this connectron are 763.283 KB and 763.304 KB with a length of 0.022 KB. (38) The connectron that has the identifier 6773 is on chromosome 1 in the positive direction. The genomic start and stop positions of this connectron are 763.306 KB and 763.321 KB with a length of 0.016 KB. (39) The connectron that has the identifier 6774 is on chromosome 1 in the positive direction. The genomic start and stop positions of this connectron are 763.346 KB and 763.360 KB with a length of 0.015 KB. (40) The connectron that has the identifier 6777 is on chromosome 1 in the positive direction. The genomic start and stop positions of this connectron are 763.362 KB and 763.376 KB with a length of 0.015 KB. (41) The connectron that has the identifier 6781 is on chromosome 1 in the negative direction. The genomic start and stop positions of this connectron are 763.986 KB and 763.965 KB with a length of 0.022 KB. (42) The connectron that has the identifier 6782 is on chromosome 1 in the positive direction. The genomic start and stop positions of this connectron are 763.965 KB and 763.995 KB with a length of 0.031 KB. (43) The connectron that has the identifier 6783 is on chromosome 1 in the negative direction. The genomic start and stop positions of this connectron are 764.011 KB and 763.990 KB with a length of 0.022 KB. (44) The connectron that has the identifier 6784 is on chromosome 1 in the positive direction. The genomic start and stop positions of this connectron are 764.000 KB and 764.015 KB with a length of 0.016 KB. (45) The connectron that has the identifier 6785 is on chromosome 1 in the negative direction. The genomic start and stop positions of this connectron are 764.089 KB and 764.075 KB with a length of 0.015 KB. (46) The connectron that has the identifier 6786 is on chromosome 1 in the negative direction. The genomic start and stop positions of this connectron are 764.108 KB and 764.093 KB with a length of 0.016 KB. (47) The connectron that has the identifier 6787 is on chromosome 1 in the positive direction. The genomic start and stop positions of this connectron are 764.156 KB and 764.171 KB with a length of 0.016 KB. (48) The connectron that has the identifier 6788 is on chromosome 1 in the negative direction. The genomic start and stop positions of this connectron are 764.177 KB and 764.156 KB with a length of 0.022 KB. (49) The connectron that has the identifier 6790 is on chromosome 1 in the negative direction. The genomic start and stop positions of this connectron are 764.195 KB and 764.181 KB with a length of 0.015 KB. (50) The connectron that has the identifier 6792 is on chromosome 1 in the positive direction. The genomic start and stop positions of this connectron are 764.181 KB and 764.204 KB with a length of 0.024 KB. (51) The connectron that has the identifier 6793 is on chromosome 1 in the positive direction. The genomic start and stop positions of this connectron are 764.213 KB and 764.227 KB with a length of 0.015 KB. (52) The connectron that has the identifier 6800 is on chromosome 1 in the positive direction. The genomic start and stop positions of this connectron are 764.791 KB and 764.808 KB with a length of 0.018 KB. (53) The connectron that has the identifier 6801 is on chromosome 1 in the negative direction. The genomic start and stop positions of this connectron are 764.813 KB and 764.794 KB with a length of 0.020 KB. (54) The connectron that has the identifier 6803 is on chromosome 1 in the positive direction. The genomic start and stop positions of this connectron are 764.810 KB and 764.826 KB with a length of 0.017 KB. (55) The connectron that has the identifier 6804 is on chromosome 1 in the negative direction. The genomic start and stop positions of this connectron are 764.832 KB and 764.818 KB with a length of 0.015 KB. (56) The connectron that has the identifier 6805 is on chromosome 1 in the positive direction. The genomic start and stop positions of this connectron are 764.831 KB and 764.853 KB with a length of 0.023 KB. (57) The connectron that has the identifier 6806 is on chromosome 1 in the positive direction. The genomic start and stop positions of this connectron are 764.856 KB and 764.870 KB with a length of 0.015 KB. (58) The connectron that has the identifier 6807 is on chromosome 1 in the negative direction. The genomic start and stop positions of this connectron are 764.998 KB and 764.984 KB with a length of 0.015 KB. (59) The connectron that has the identifier 6809 is on chromosome 1 in the negative direction. The genomic start and stop positions of this connectron are 765.014 KB and 765.000 KB with a length of 0.015 KB. (60) The connectron that has the identifier 6811 is on chromosome 1 in the negative direction. The genomic start and stop positions of this connectron are 765.446 KB and 765.431 KB with a length of 0.016 KB. (61) The connectron that has the identifier 6812 is on chromosome 1 in the negative direction. The genomic start and stop positions of this connectron are 765.473 KB and 765.456 KB with a length of 0.018 KB. (62) The connectron that has the identifier 6813 is on chromosome 1 in the negative direction. The genomic start and stop positions of this connectron are 765.494 KB and 765.479 KB with a length of 0.016 KB. (63) The connectron that has the identifier 6821 is on chromosome 1 in the negative direction. The genomic start and stop positions of this connectron are 766.653 KB and 766.635 KB with a length of 0.019 KB. (64) The connectron that has the identifier 6823 is on chromosome 1 in the negative direction. The genomic start and stop positions of this connectron are 766.675 KB and 766.655 KB with a length of 0.021 KB. (65) The connectron that has the identifier 6838 is on chromosome 1 in the positive direction. The genomic start and stop positions of this connectron are 770.226 KB and 770.240 KB with a length of 0.015 KB. (66) The connectron that has the identifier 6839 is on chromosome 1 in the positive direction. The genomic start and stop positions of this connectron are 770.251 KB and 770.265 KB with a length of 0.015 KB. (67) The connectron that has the identifier 6841 is on chromosome 1 in the positive direction. The genomic start and stop positions of this connectron are 770.347 KB and 770.363 KB with a length of 0.017 KB. (68) The connectron that has the identifier 6842 is on chromosome 1 in the positive direction. The genomic start and stop positions of this connectron are 770.365 KB and 770.379 KB with a length of 0.015 KB. (69) The connectron that has the identifier 6845 is on chromosome 1 in the negative direction. The genomic start and stop positions of this connectron are 772.086 KB and 771.564 KB with a length of 0.523 KB. (70) The connectron that has the identifier 6851 is on chromosome 1 in the positive direction. The genomic start and stop positions of this connectron are 773.399 KB and 773.483 KB with a length of 0.085 KB.

### Addition of a Synthetic Virtual Connectron of Type 15 from Table 2

A synthetic virtual connectron of type 15 in which the C1 and C2 are natural elements, the T1 is a natural element and the T2 is a synthetic element can be introduced into the P. aeruginosa genome by modifying the DNA double-strand sequences at 4838.155 kb through 4838.323kb for a length of 0.169 kb.

To create this example of a synthetic connectron of type 14 the T2 sequence for natural connectron 23295 (old position 4848.679 kb through 4939.847 kb) was copied into the new position stated above.

Connectron 28739 is an example of a synthetic transient connectron. It is described as

| | | C1/C2 | | | T1-T2 | | |
|---|---|---|---|---|---|---|---|
| Global_id | Chromosome | C1_Id | C2_Id | Chromosome | T1_Id | T2_Id | Connectron_Type |
| 28739 | 1 | 53464 | 53464 | 1 | 53531 | 53569 | transient |

The C1/C2 source of the transient connectron 28739 is represented in table 1 as

| Type | Num | Jobno | Chr | Start | Stop | Length | GeneName |
|---|---|---|---|---|---|---|---|
| CPT | 53464 | 1 | 1 | 4832.718 | 4832.838 | .121 | −−> \|\|\|\|\|\|\|\|\|\|\|\|\|\|\|\|\|\|\|\|\|\|\|\|\|\|\|\|\| |

The “Type” descriptor of this transient C1/C2 connectron source is “CPT”. The letter “P” indicates that the C1/C2 connectron source occurs on the positive strand of the double-stranded DNA of the genome. The letter “N” in this place would indicate a C1/C2 connectron source on the negative strand of the genomic DNA. The letter “T” in this descriptor indicates a “transient” connectron. Similarly, the letter “P” would indicate a permanent connectron that is shown in a later example. The “Start”, “Stop” and “Length” descriptors throughout these examples are given in kilo-bases (KB).

The T1-T2 target of the transient connectron 28739 as shown in table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TP | 53531 | 1 | 1 | 4836.528 | 4836.721 | .194 | *−* |
| CPT | 53532 | 1 | 1 | 4836.728 | 4836.746 | .019 | OS−> \| |
| TP | 53533 | 1 | 1 | 4836.728 | 4836.746 | .019 | *−+ |
| TN | 53534 | 1 | 1 | 4836.728 | 4836.779 | .052 | *−+ |
| CPT | 53535 | 1 | 1 | 4836.748 | 4836.779 | .032 | OS−> \| |
| TP | 53536 | 1 | 1 | 4836.748 | 4836.779 | .032 | *−+ |
| CPT | 53537 | 1 | 1 | 4836.781 | 4836.821 | .041 | OS−> \| |
| TP | 53538 | 1 | 1 | 4836.781 | 4836.821 | .041 | *−+ |
| TN | 53539 | 1 | 1 | 4836.781 | 4836.918 | .138 | *−+ |
| TP | 53540 | 1 | 1 | 4836.840 | 4836.855 | .016 | *−+ |
| CPT | 53541 | 1 | 1 | 4836.877 | 4836.918 | .042 | OS−> \| |
| TP | 53542 | 1 | 1 | 4836.877 | 4836.918 | .042 | *−+ |
| TP | 53543 | 1 | 1 | 4836.940 | 4836.957 | .018 | *−+ |
| CPT | 53544 | 1 | 1 | 4836.940 | 4836.982 | .043 | OS−> \| |
| CPT | 53545 | 1 | 1 | 4836.990 | 4837.123 | .134 | OS−> \| |
| TP | 53546 | 1 | 1 | 4836.990 | 4837.123 | .134 | *−+ |
| TN | 53547 | 1 | 1 | 4836.990 | 4837.153 | .164 | *−+ |
| CPT | 53548 | 1 | 1 | 4837.126 | 4837.148 | .023 | −−> \| |
| TP | 53549 | 1 | 1 | 4837.129 | 4837.153 | .025 | *−+ |
| TP | 53549a | 1 | 1 | 4838.155 | 4838.323 | .169 | *−* |

The “Type” descriptor of the T1 (Id number 53531) is “TP” showing that is T1 target is on the positive strand of the double-stranded genomic DNA. Because the T1 and the T2 targets have to be on the same strand, the T2 target (Id number 53549a) also has the type descriptor “TP”.

The T1-T2 loop diagram above can be read by tracing the path from the * of the T1 to the right to the appropriate *, then by tracing a path down the diagram to the first *, then by tracing a path left to the * of the T2. When horizontal lines (indicated by −) intersect with other vertical lines (indicated by |) then the symbol at that point is changed to a +. When the T1-T2 loop does not contain an groups of genes then the vertical symbol (indicated by | or +) is changed to @ as shown below

| Type | Num | Jobno | Chr | Start | Stop | Length | GeneName |
|---|---|---|---|---|---|---|---|
| TN | xx | 1 | 1 | 278.386 | 279.148 | .763 | *---* |
| XX | xx | 1 | 1 | 278.387 | 278.416 | .030 | * @ |
| XX | xx | 1 | 1 | 278.421 | 278.450 | .030 | * @ |
| TP | xx | 1 | 1 | 278.452 | 278.892 | .441 | *---* |

Since this synthetic connectron is a virtual connectron there are no gene groups that are controlled by the existence of this connectron. Instead the expression of the C1/C2 sources of six one-shot connectrons (type designator “CPT” for Id numbers 53532, 53535, 53537, 53541, 53544, and 53544) and one transient connectron (type designator “CPT” for Id numbers 53548) are controlled by this connectron.

All of the data for the transient connectron 28739 are pulled together in the following table that is the “terse” description of the connectron.

| Connectron Relationships - | Global_Id | Type |
|---|---|---|
| | 28739 | transient |

| | | Chro- | | | | |
|---|---|---|---|---|---|---|
| Control | Direc- | mo- | C1/ | | | |
| Sequences - | tion | some | C2_Id | Start | Stop | Length |
| | posi- | 1 | 53464 | 4832.718 | 4832.838 | .121 |
| | tive | | | | | |

| Trigger Gene - | Name | COG_Id | Start | Stop | Length |
|---|---|---|---|---|---|
| | pctC | COG0840 | 4831.371 | 4833.269 | 1.899 |

| | | Chro- | | | | |
|---|---|---|---|---|---|---|
| Target | Direc- | mo- | | | | |
| Sequences - | tion | some | | Start | Stop | Length |
| | | | T1_Id | | | |
| | posi- | 1 | 53531 | 4836.528 | 4836.721 | .194 |
| | tive | | | | | |
| | | | T2_Id | | | |
| | | | 53549a | 4838.155 | 4838.323 | .169 |

Controlled Genes

none

Controlled Connectrons

| | Chro- | | | | | |
|---|---|---|---|---|---|---|
| Local_{—} | mo- | C1/ | Direc- | | | |
| Id | some | C2_Id | tion | Start | Stop | Length |
| 1 | 1 | 53532 | positive | 4836.728 | 4836.746 | .019 |
| 2 | 1 | 53535 | positive | 4836.748 | 4836.779 | .032 |
| 3 | 1 | 53537 | positive | 4836.781 | 4836.821 | .041 |
| 4 | 1 | 53541 | positive | 4836.877 | 4836.918 | .042 |
| 5 | 1 | 53544 | positive | 4836.940 | 4836.982 | .043 |
| 6 | 1 | 53545 | positive | 4836.990 | 4837.123 | .134 |
| 7 | 1 | 53548 | positive | 4837.126 | 4837.148 | .023 |
| 8 | 1 | 53551 | positive | 4837.419 | 4837.448 | .030 |
| 9 | 1 | 53552 | positive | 4837.572 | 4837.622 | .051 |
| 10 | 1 | 53554 | positive | 4837.962 | 4837.999 | .038 |
| 11 | 1 | 53557 | positive | 4838.217 | 4838.258 | .042 |
| 12 | 1 | 53559 | positive | 4838.295 | 4838.362 | .068 |
| 13 | 1 | 53563 | positive | 4838.457 | 4838.476 | .020 |
| 14 | 1 | 53564 | positive | 4838.478 | 4838.645 | .168 |
| 15 | 1 | 53567 | positive | 4838.654 | 4838.847 | .194 |

The verbose description of the transient connectron 28739 is:

In the Pseudomonas aeruginosa PA01, complete genome the transient connectron number 23295 is generated by the control sequence (C1/C2) whose identifier number is 53464. This control sequence is on the positive strand of the genomic DNA of chromosome 1. The genomic start and stop positions of this control sequence are 4832.718 KB and 4832.838 KB with a length of 0.121 KB. Expression of the RNA for this connectron is triggered by the promotion of the gene whose name is pctc and whose COG (Cluster of Orthologous Genes) identifier is COG0840. The genomic start and stop positions of this gene are 4831.371 KB and 4833.269 KB and with a length of 1.899 KB. This connectron causes stabilization of a loop of DNA. The target sequences (T1-T2) are on the positive strand of the genomic DNA on chromosome 1. The identifier number of the initiating target sequence (T1) is 53531. The genomic start and stop positions of this initiating target sequence are 4836.528 KB and 4836.721 KB with a length of 0.194 KB. The identifier number of the terminating target sequence (T2) is 53569. The genomic start and stop positions of this terminating target sequence are 4838.155 KB and 4838.323 KB with a length of 0.169 KB.

This connectron controls the modulation of the expression of the following gene(s): none

This connectron controls the turning off of the expression of the following connectron(s): (1) The connectron that has the identifier 53532 is on chromosome 1 in the positive direction. The genomic start and stop positions of this connectron are 4836.728 KB and 4836.746 KB with a length of 0.019 KB. (2) The connectron that has the identifier 53535 is on chromosome 1 in the positive direction. The genomic start and stop positions of this connectron are 4836.748 KB and 4836.779 KB with a length of 0.032 KB. (3) The connectron that has the identifier 53537 is on chromosome 1 in the positive direction. The genomic start and stop positions of this connectron are 4836.781 KB and 4836.821 KB with a length of 0.041 KB. (4) The connectron that has the identifier 53541 is on chromosome 1 in the positive direction. The genomic start and stop positions of this connectron are 4836.877 KB and 4836.918 KB with a length of 0.042 KB. (5) The connectron that has the identifier 53544 is on chromosome 1 in the positive direction. The genomic start and stop positions of this connectron are 4836.940 KB and 4836.982 KB with a length of 0.043 KB. (6) The connectron that has the identifier 53545 is on chromosome 1 in the positive direction. The genomic start and stop positions of this connectron are 4836.990 KB and 4837.123 KB with a length of 0.134 KB. (7) The connectron that has the identifier 53548 is on chromosome 1 in the positive direction. The genomic start and stop positions of this connectron are 4837.126 KB and 4837.148 KB with a length of 0.023 KB. (8) The connectron that has the identifier 53551 is on chromosome 1 in the positive direction. The genomic start and stop positions of this connectron are 4837.419 KB and 4837.448 KB with a length of 0.030 KB. (9) The connectron that has the identifier 53552 is on chromosome 1 in the positive direction. The genomic start and stop positions of this connectron are 4837.572 KB and 4837.622 KB with a length of 0.051 KB. (10) The connectron that has the identifier 53554 is on chromosome 1 in the positive direction. The genomic start and stop positions of this connectron are 4837.962 KB and 4837.999 KB with a length of 0.038 KB. (11) The connectron that has the identifier 53557 is on chromosome 1 in the positive direction. The genomic start and stop positions of this connectron are 4838.217 KB and 4838.258 KB with a length of 0.042 KB. (12) The connectron that has the identifier 53559 is on chromosome 1 in the positive direction. The genomic start and stop positions of this connectron are 4838.295 KB and 4838.362 KB with a length of 0.068 KB. (13) The connectron that has the identifier 53563 is on chromosome 1 in the positive direction. The genomic start and stop positions of this connectron are 4838.457 KB and 4838.476 KB with a length of 0.020 KB. (14) The connectron that has the identifier 53564 is on chromosome 1 in the positive direction. The genomic start and stop positions of this connectron are 4838.478 KB and 4838.645 KB with a length of 0.168 KB. (15) The connectron that has the identifier 53567 is on chromosome 1 in the positive direction. The genomic start and stop positions of this connectron are 4838.654 KB and 4838.847 KB with a length of 0.194 KB.

### Synthetic Connectron Formed by a DBP

A synthetic connectron can be introduced into the V. cholerae genome by introducing a 12-domain zinc-finger protein that recognizes the sequence

tccccatgag catagatatg cagqtaggcq gcaagt

Connectron 7580 is an example of a synthetic transient connectron.

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 7580 | 1 | Synthetic | DBP | 1 | 607 | 646 | synthetic |

The T1-T2 target of the transient connectron 7580 as shown in Table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TN | 607 | 1 | 1 | 952.642 | 952.777 | .136 | *−++++*+++*+++++++*+*++++**++++ |
| TP | 608 | 1 | 1 | 952.648 | 952.777 | .130 | *−++++−+++−*+++*+*−++*++++−++++ |
| GG | 609 | 1 | 1 | 952.846 | 954.393 | 1.548 | Group0127 \|\|\|\| \|\|\| \|\|\| \| \|\| \|\|\|\| \|\|\|\| |
| TN | 610 | 1 | 1 | 954.486 | 954.587 | .102 | *−++++*+++*−+++−+−*+*−++*+**+++ |
| TP | 611 | 1 | 1 | 954.486 | 954.587 | .102 | *−+++++++++**++*+*++−**+−++−+*+ |
| CNT | 612 | 1 | 1 | 954.497 | 954.589 | .093 | OS−> \|\|\|\|\|\|\|\|\|\| \|\|\|\|\|\|\| \| \| \|\| \| \| |
| CPT | 613 | 1 | 1 | 954.503 | 954.520 | .018 | −−> \|\|\|\|\|\|\|\|\|\| \|\|\|\|\|\|\| \| \| \|\| \| \| |
| CPT | 614 | 1 | 1 | 954.522 | 954.588 | .067 | −−> \|\|\|\|\|\|\|\|\|\| \|\|\|\|\|\|\| \| \| \|\| \| \| |
| TP | 615 | 1 | 1 | 954.601 | 954.658 | .058 | *−++++++++++*+++++++*+*+*++*++−+−* |
| TN | 616 | 1 | 1 | 954.602 | 954.658 | .057 | *−+++++++++++++++++*+++++*+***− |
| CNT | 617 | 1 | 1 | 954.616 | 954.658 | .043 | −−> \|\|\|\|\|\|\|\|\|\|\|\|\|\|\|\|\| \|\|\|\|\| \|\| \| |
| CPT | 618 | 1 | 1 | 954.624 | 954.639 | .016 | −−> \|\|\|\|\|\|\|\|\|\|\|\|\|\|\|\|\| \|\|\|\|\| \|\| \| |
| CPT | 619 | 1 | 1 | 954.641 | 954.658 | .018 | −−> \|\|\|\|\|\|\|\|\|\|\|\|\|\|\|\|\| \|\|\|\|\| \|\| \| |
| GG | 620 | 1 | 1 | 954.684 | 958.635 | 3.951 | Group0128 \|\|\|\|\|\|\|\|\|\|\|\|\|\|\|\|\| \|\|\|\|\| \|\| \| |
| GP | 621 | 1 | 1 | 955.956 | 956.342 | .387 | VC0897 \|\|\|\|\|\|\|\|\|\|\|\|\|\|\|\|\| \|\|\|\|\| \|\| \| |
| CPT | 622 | 1 | 1 | 956.873 | 956.887 | .015 | −−> \|\|\|\|\|\|\|\|\|\|\|\|\|\|\|\|\| \|\|\|\|\| \|\| \| |
| CPT | 623 | 1 | 1 | 956.896 | 956.910 | .015 | −−> \|\|\|\|\|\|\|\|\|\|\|\|\|\|\|\|\| \|\|\|\|\| \|\| \| |
| TN | 624 | 1 | 1 | 958.089 | 958.103 | .015 | *−+*\|\|\|\|\|\|\|\|\|\|\|\|\|\|\| \|\|\|\|\| \|\| \| |
| GG | 625 | 1 | 1 | 958.840 | 972.992 | 14.152 | Group0129 \| \|\|\|\|\|\|\|\|\|\|\|\|\|\|\| \|\|\|\|\| \|\| \| |
| GP | 626 | 1 | 1 | 971.304 | 972.992 | 1.689 | VC0911 \| \|\|\|\|\|\|\|\|\|\|\|\|\|\|\| \|\|\|\|\| \|\| \| |
| TP | 627 | 1 | 1 | 973.170 | 973.250 | .081 | *−+*\|\|\|\|\|\|\|\|\|\|\|\|\|\|\| \|\|\|\|\| \|\| \| |
| GG | 628 | 1 | 1 | 973.171 | 973.763 | .592 | Group0130 \|\|\|\|\|\|\|\|\|\|\|\|\|\|\|\|\| \|\|\|\|\| \|\| \| |
| CPT | 629 | 1 | 1 | 973.339 | 973.434 | .096 | OS−> \|\|\|\|\|\|\|\|\|\|\|\|\|\|\|\|\| \|\|\|\|\| \|\| \| |
| CPT | 630 | 1 | 1 | 973.508 | 973.603 | .096 | OS−> \|\|\|\|\|\|\|\|\|\|\|\|\|\|\|\|\| \|\|\|\|\| \|\| \| |
| TP | 631 | 1 | 1 | 973.693 | 973.773 | .081 | *−+*\|\|\|\|\|\|\|\|\|\|\|\|\|\|\| \|\|\|\|\| \|\| \| |
| GG | 632 | 1 | 1 | 973.767 | 996.501 | 22.734 | Group0131 \| \|\|\|\|\|\|\|\|\|\|\|\|\|\|\| \|\|\|\|\| \|\| \| |
| GP | 633 | 1 | 1 | 974.049 | 978.217 | 4.169 | VC0913 \| \|\|\|\|\|\|\|\|\|\|\|\|\|\|\| \|\|\|\|\| \|\| \| |
| CPT | 634 | 1 | 1 | 977.711 | 977.725 | .015 | −−> \| \|\|\|\|\|\|\|\|\|\|\|\|\|\|\| \|\|\|\|\| \|\| \| |
| CPT | 635 | 1 | 1 | 977.732 | 977.746 | .015 | −−> \| \|\|\|\|\|\|\|\|\|\|\|\|\|\|\| \|\|\|\|\| \|\| \| |
| GP | 636 | 1 | 1 | 986.166 | 990.921 | 4.756 | VC0923 \| \|\|\|\|\|\|\|\|\|\|\|\|\|\|\| \|\|\|\|\| \|\| \| |
| CPT | 637 | 1 | 1 | 986.862 | 986.877 | .016 | −−> \| \|\|\|\|\|\|\|\|\|\|\|\|\|\|\| \|\|\|\|\| \|\| \| |
| CPT | 638 | 1 | 1 | 986.885 | 986.899 | .015 | −−> \| \|\|\|\|\|\|\|\|\|\|\|\|\|\|\| \|\|\|\|\| \|\| \| |
| TN | 639 | 1 | 1 | 996.556 | 996.577 | .022 | *−+**\|\|\|\|\|\|\|\|\|\|\|\|\|\| \|\|\|\|\| \|\| \| |
| TP | 640 | 1 | 1 | 996.556 | 996.577 | .022 | *−++−+++*++++++++++*\|\|\|\|\| \|\| \| |
| GG | 641 | 1 | 1 | 996.656 | 1005.792 | 9.136 | Group0132 \|\| \|\|\| \|\|\|\|\|\|\|\|\|\|\|\|\|\|\|\| \|\| \| |
| GP | 642 | 1 | 1 | 1002.462 | 1005.792 | 3.331 | VC0937 \|\| \|\|\| \|\|\|\|\|\|\|\|\|\|\|\|\|\|\|\| \|\| \| |
| TP | 643 | 1 | 1 | 1005.810 | 1005.874 | .065 | *−++*+++*++*+*+++++++++++*++*+* |
| CNT | 644 | 1 | 1 | 1005.810 | 1005.921 | .112 | OS−> \|\|\|\|\|\|\|\|\| \| \|\|\|\|\|\|\|\|\|\|\|\|\|\|\|\|\| |
| CPT | 645 | 1 | 1 | 1005.810 | 1005.926 | .117 | −−> \|\|\|\|\|\|\|\|\| \| \|\|\|\|\|\|\|\|\|\|\|\|\|\|\|\|\| |
| TN | 646 | 1 | 1 | 1005.810 | 1005.926 | .117 | *−++++*++++*+*+++++++++*+++*+++ |

Group0127 through Group0132 are described in Table 2 as

| Gene_Name | COG_Id | | Chromosome | Direction | Start | Stop | Length |
|---|---|---|---|---|---|---|---|
| Group0127 | | | | | | | |
| VC0894 | COG0607 | P | 1 | positive | 952.846 | 954.393 | 1.548 |
| Group0128 | | | | | | | |
| VC0895 | | | 1 | negative | 954.684 | 954.860 | .177 |
| VC0896 | COG0583 | K | 1 | positive | 955.014 | 955.934 | .921 |
| VC0897 | COG2363 | S | 1 | positive | 955.956 | 956.342 | .387 |
| VC0898 | COG0258 | L | 1 | negative | 956.372 | 957.223 | .852 |
| VC0899 | COG1611 | R | 1 | negative | 957.262 | 958.635 | 1.374 |
| Group0129 | | | | | | | |
| VC0900 | COG2199 | T | 1 | negative | 958.840 | 960.414 | 1.575 |
| VC0901 | COG0457 | R | 1 | negative | 960.516 | 962.777 | 2.262 |
| VC0902 | COG0780 | R | 1 | negative | 962.792 | 963.655 | .864 |
| VC0903 | | | 1 | positive | 963.686 | 964.282 | .597 |
| VC0904 | | | 1 | positive | 964.284 | 965.060 | .777 |
| VC0905 | COG1464 | M | 1 | negative | 965.193 | 966.020 | .828 |
| VC0906 | COG2011 | R | 1 | negative | 966.039 | 967.740 | 1.702 |
| VC0908 | COG0241 | E | 1 | positive | 967.976 | 968.536 | .561 |
| VC0909 | COG1609 | K | 1 | positive | 968.601 | 969.551 | .951 |
| VC0910 | COG1263 | G | 1 | positive | 969.788 | 971.224 | 1.437 |
| VC0911 | COG0366 | G | 1 | positive | 971.304 | 972.992 | 1.689 |
| Group0130 | | | | | | | |
| tRNA-Tyr-2 | | | 1 | positive | 973.171 | 973.255 | .085 |
| tRNA-Tyr-3 | | | 1 | positive | 973.340 | 973.424 | .085 |
| tRNA-Tyr-4 | | | 1 | positive | 973.509 | 973.593 | .085 |
| tRNA-Tyr-5 | | | 1 | positive | 973.679 | 973.763 | .085 |
| Group0131 | | | | | | | |
| VC0912 | | | 1 | positive | 973.767 | 973.862 | .096 |
| VC0913 | COG0845 | Q | 1 | positive | 974.049 | 978.217 | 4.169 |
| VC0915 | | | 1 | positive | 978.272 | 978.382 | .111 |
| VC0916 | COG0394 | T | 1 | positive | 978.644 | 979.144 | .501 |
| VC0917 | COG0381 | M | 1 | positive | 979.457 | 980.575 | 1.119 |
| VC0918 | COG0677 | M | 1 | positive | 980.595 | 986.155 | 5.561 |
| VC0923 | COG1045 | E | 1 | positive | 986.166 | 990.921 | 4.756 |
| VC0928 | | | 1 | positive | 991.117 | 991.932 | .816 |
| VC0929 | | | 1 | negative | 992.138 | 993.364 | 1.227 |
| VC0930 | | | 1 | positive | 993.628 | 996.501 | 2.874 |
| Group0132 | | | | | | | |
| VC0931 | COG3307 | M | 1 | negative | 996.656 | 997.972 | 1.317 |
| VC0932 | | | 1 | negative | 998.148 | 998.752 | .605 |
| VC0934 | COG2148 | M | 1 | positive | 999.242 | 1000.639 | 1.398 |
| VC0935 | | | 1 | positive | 1000.710 | 1001.906 | 1.197 |
| VC0936 | COG1596 | M | 1 | positive | 1001.910 | 1002.437 | .528 |
| VC0937 | COG3206 | N | 1 | positive | 1002.462 | 1005.792 | 3.331 |

All of the data for the transient connectron 7580 are pulled together in the following table that is the “terse” description of the connectron.

| Connectron Relationships - | Global_Id | Type |
|---|---|---|
| | 7580 | synthetic |

Control Sequences - Synthetic DNA Binding
Protein that recognizes the sequence
tccccatgag catagatatg caggtaggcg gcaagt

| | | Chro- | | | | |
|---|---|---|---|---|---|---|
| Target | | mo- | | | | |
| Sequences - | Direction | some | | Start | Stop | Length |
| | | | T1_Id | | | |
| | negative | 1 | 607 | 952.777 | 952.642 | .136 |
| | | | T2_Id | | | |
| | | | 646 | 1005.926 | 1005.810 | .117 |

Controlled Genes

| Local_Id | Chromosome | Group | Name | COG_Id | Direction | Start | Stop | Length |
|---|---|---|---|---|---|---|---|---|
| 1 | 1 | Group0127 | VC0894 | COG0607 | positive | 952.846 | 954.393 | 1.548 |
| 2 | 1 | Group0128 | VC0895 | | positive | 954.684 | 958.635 | 3.951 |
| 3 | 1 | Group0128 | VC0896 | COG0583 | positive | 955.014 | 955.934 | .921 |
| 4 | 1 | Group0128 | VC0897 | COG2363 | positive | 955.956 | 956.342 | .387 |
| 5 | 1 | Group0128 | VC0898 | COG0258 | negative | 957.223 | 956.372 | .852 |
| 6 | 1 | Group0128 | VC0899 | COG1611 | negative | 958.635 | 957.262 | 1.374 |
| 7 | 1 | Group0129 | VC0900 | COG2199 | positive | 958.840 | 972.992 | 14.152 |
| 8 | 1 | Group0129 | VC0901 | COG0457 | negative | 962.777 | 960.516 | 2.262 |
| 9 | 1 | Group0129 | VC0902 | COG0780 | negative | 963.655 | 962.792 | .864 |
| 10 | 1 | Group0129 | VC0903 | | positive | 963.686 | 964.282 | .597 |
| 11 | 1 | Group0129 | VC0904 | | positive | 964.284 | 965.060 | .777 |
| 12 | 1 | Group0129 | VC0905 | COG1464 | negative | 966.020 | 965.193 | .828 |
| 13 | 1 | Group0129 | VC0906 | COG2011 | negative | 967.740 | 966.039 | 1.702 |
| 14 | 1 | Group0129 | VC0908 | COG0241 | positive | 967.976 | 968.536 | .561 |
| 15 | 1 | Group0129 | VC0909 | COG1609 | positive | 968.601 | 969.551 | .951 |
| 16 | 1 | Group0129 | VC0910 | COG1263 | positive | 969.788 | 971.224 | 1.437 |
| 17 | 1 | Group0129 | VC0911 | COG0366 | positive | 971.304 | 972.992 | 1.689 |
| 18 | 1 | Group0130 | tRNA-Tyr- | | positive | 973.171 | 973.763 | .592 |
| 19 | 1 | Group0131 | VC0912 | | positive | 973.767 | 996.501 | 22.734 |
| 20 | 1 | Group0131 | VC0913 | COG0845 | positive | 974.049 | 978.217 | 4.169 |
| 21 | 1 | Group0131 | VC0915 | | positive | 978.272 | 978.382 | .111 |
| 22 | 1 | Group0131 | VC0916 | COG0394 | positive | 978.644 | 979.144 | .501 |
| 23 | 1 | Group0131 | VC0917 | COG0381 | positive | 979.457 | 980.575 | 1.119 |
| 24 | 1 | Group0131 | VC0918 | COG0677 | positive | 980.595 | 986.155 | 5.561 |
| 25 | 1 | Group0131 | VC0923 | COG1045 | positive | 986.166 | 990.921 | 4.756 |
| 26 | 1 | Group0131 | VC0928 | | positive | 991.117 | 991.932 | .816 |
| 27 | 1 | Group0131 | VC0929 | | negative | 993.364 | 992.138 | 1.227 |
| 28 | 1 | Group0131 | VC0930 | | positive | 993.628 | 996.501 | 2.874 |
| 29 | 1 | Group0132 | VC0931 | COG3307 | positive | 996.656 | 1005.792 | 9.136 |
| 30 | 1 | Group0132 | VC0932 | | negative | 998.752 | 998.148 | .605 |
| 31 | 1 | Group0132 | VC0934 | COG2148 | positive | 999.242 | 1000.639 | 1.398 |
| 32 | 1 | Group0132 | VC0935 | | positive | 1000.710 | 1001.906 | 1.197 |
| 33 | 1 | Group0132 | VC0936 | COG1596 | positive | 1001.910 | 1002.437 | .528 |
| 34 | 1 | Group0132 | VC0937 | COG3206 | positive | 1002.462 | 1005.792 | 3.331 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 612 | negative | 954.589 | 954.497 | .093 |
| 2 | 1 | 613 | positive | 954.503 | 954.520 | .018 |
| 3 | 1 | 614 | positive | 954.522 | 954.588 | .067 |
| 4 | 1 | 617 | negative | 954.658 | 954.616 | .043 |
| 5 | 1 | 618 | positive | 954.624 | 954.639 | .016 |
| 6 | 1 | 619 | positive | 954.641 | 954.658 | .018 |
| 7 | 1 | 622 | positive | 956.873 | 956.887 | .015 |
| 8 | 1 | 623 | positive | 956.896 | 956.910 | .015 |
| 9 | 1 | 629 | positive | 973.339 | 973.434 | .096 |
| 10 | 1 | 630 | positive | 973.508 | 973.603 | .096 |
| 11 | 1 | 634 | positive | 977.711 | 977.725 | .015 |
| 12 | 1 | 635 | positive | 977.732 | 977.746 | .015 |
| 13 | 1 | 637 | positive | 986.862 | 986.877 | .016 |
| 14 | 1 | 638 | positive | 986.885 | 986.899 | .015 |
| 15 | 1 | 644 | negative | 1005.921 | 1005.810 | .112 |
| 16 | 1 | 645 | positive | 1005.810 | 1005.926 | .117 |

The verbose description of the synthetic connectron 7580 is:

In the Vibrio cholerae chromosome I, complete chromosome the synthetic connectron number 7580 is generated by a synthetic DNA binding protein that recognizes the sequence tccccatgagcatagatatgcaggtaggcggcaagt. This connectron causes stabilization of a loop of DNA. The target sequences (T1-T2) are on the negative strand of the genomic DNA on chromosome 1. The identifier number of the initiating target sequence (T1) is 607. The genomic start and stop positions of this initiating target sequence are 952.777 KB and 952.642 KB with a length of 0.136 KB. The identifier number of the terminating target sequence (T2) is 646. The genomic start and stop positions of this terminating target sequence are 1005.926 KB and 1005.810 KB with a length of 0.117 KB.

This connectron controls the modulation of the expression of the following gene(s): (1) The gene that has the name VC0894 and with a COG identifier of COG0607. The genomic start and stop positions of this gene are 952.846 KB and 954.393 KB with a length of 1.548 KB. (2) The gene that has the name VC0895. The genomic start and stop positions of this gene are 954.684 KB and 958.635 KB with a length of 3.951 KB. (3) The gene that has the name VC0896 and with a COG identifier of COG0583. The genomic start and stop positions of this gene are 955.014 KB and 955.934 KB with a length of 0.921 KB. (4) The gene that has the name VC0897 and with a COG identifier of COG2363. The genomic start and stop positions of this gene are 955.956 KB and 956.342 KB with a length of 0.387 KB. (5) The gene that has the name VC0898 and with a COG identifier of COG0258. The genomic start and stop positions of this gene are 957.223 KB and 956.372 KB with a length of 0.852 KB. (6) The gene that has the name VC0899 and with a COG identifier of COG1611. The genomic start and stop positions of this gene are 958.635 KB and 957.262 KB with a length of 1.374 KB. (7) The gene that has the name VC0900 and with a COG identifier of COG2199. The genomic start and stop positions of this gene are 958.840 KB and 972.992 KB with a length of 14.152 KB. (8) The gene that has the name VC0901 and with a COG identifier of COG0457. The genomic start and stop positions of this gene are 962.777 KB and 960.516 KB with a length of 2.262 KB. (9) The gene that has the name VC0902 and with a COG identifier of COG0780. The genomic start and stop positions of this gene are 963.655 KB and 962.792 KB with a length of 0.864 KB. (10) The gene that has the name VC0903. The genomic start and stop positions of this gene are 963.686 KB and 964.282 KB with a length of 0.597 KB. (11) The gene that has the name VC0904. The genomic start and stop positions of this gene are 964.284 KB and 965.060 KB with a length of 0.777 KB. (12) The gene that has the name VC0905 and with a COG identifier of COG1464. The genomic start and stop positions of this gene are 966.020 KB and 965.193 KB with a length of 0.828 KB. (13) The gene that has the name VC0906 and with a COG identifier of COG2011. The genomic start and stop positions of this gene are 967.740 KB and 966.039 KB with a length of 1.702 KB. (14) The gene that has the name VC0908 and with a COG identifier of COG0241. The genomic start and stop positions of this gene are 967.976 KB and 968.536 KB with a length of 0.561 KB. (15) The gene that has the name VC0909 and with a COG identifier of COG1609. The genomic start and stop positions of this gene are 968.601 KB and 969.551 KB with a length of 0.951 KB. (16) The gene that has the name VC0910 and with a COG identifier of COG1263. The genomic start and stop positions of this gene are 969.788 KB and 971.224 KB with a length of 1.437 KB. (17) The gene that has the name VC0911 and with a COG identifier of COG0366. The genomic start and stop positions of this gene are 971.304 KB and 972.992 KB with a length of 1.689 KB. (18) The gene that has the name tRNA-Tyr-. The genomic start and stop positions of this gene are 973.171 KB and 973.763 KB with a length of 0.592 KB. (19) The gene that has the name VC0912. The genomic start and stop positions of this gene are 973.767 KB and 996.501 KB with a length of 22.734 KB. (20) The gene that has the name VC0913 and with a COG identifier of COG0845. The genomic start and stop positions of this gene are 974.049 KB and 978.217 KB with a length of 4.169 KB. (21) The gene that has the name VC0915. The genomic start and stop positions of this gene are 978.272 KB and 978.382 KB with a length of 0.111 KB. (22) The gene that has the name VC0916 and with a COG identifier of COG0394. The genomic start and stop positions of this gene are 978.644 KB and 979.144 KB with a length of 0.501 KB. (23) The gene that has the name VC0917 and with a COG identifier of COG0381. The genomic start and stop positions of this gene are 979.457 KB and 980.575 KB with a length of 1.119 KB. (24) The gene that has the name VC0918 and with a COG identifier of COG0677. The genomic start and stop positions of this gene are 980.595 KB and 986.155 KB with a length of 5.561 KB. (25) The gene that has the name VC0923 and with a COG identifier of COG1045. The genomic start and stop positions of this gene are 986.166 KB and 990.921 KB with a length of 4.756 KB. (26) The gene that has the name VC0928. The genomic start and stop positions of this gene are 991.117 KB and 991.932 KB with a length of 0.816 KB. (27) The gene that has the name VC0929. The genomic start and stop positions of this gene are 993.364 KB and 992.138 KB with a length of 1.227 KB. (28) The gene that has the name VC0930. The genomic start and stop positions of this gene are 993.628 KB and 996.501 KB with a length of 2.874 KB. (29) The gene that has the name VC0931 and with a COG identifier of COG3307. The genomic start and stop positions of this gene are 996.656 KB and 1005.792 KB with a length of 9.136 KB. (30) The gene that has the name VC0932. The genomic start and stop positions of this gene are 998.752 KB and 998.148 KB with a length of 0.605 KB. (31) The gene that has the name VC0934 and with a COG identifier of COG2148. The genomic start and stop positions of this gene are 999.242 KB and 1000.639 KB with a length of 1.398 KB. (32) The gene that has the name VC0935. The genomic start and stop positions of this gene are 1000.710 KB and 1001.906 KB with a length of 1.197 KB. (33) The gene that has the name VC0936 and with a COG identifier of COG1596. The genomic start and stop positions of this gene are 1001.910 KB and 1002.437 KB with a length of 0.528 KB. (34) The gene that has the name VC0937 and with a COG identifier of COG3206. The genomic start and stop positions of this gene are 1002.462 KB and 1005.792 KB with a length of 3.331 KB.

This connectron controls the turning off of the expression of the following connectron(s): (1) The connectron that has the identifier 612 is on chromosome 1 in the negative direction. The genomic start and stop positions of this connectron are 954.589 KB and 954.497 KB with a length of 0.093 KB. (2) The connectron that has the identifier 613 is on chromosome 1 in the positive direction. The genomic start and stop positions of this connectron are 954.503 KB and 954.520 KB with a length of 0.018 KB. (3) The connectron that has the identifier 614 is on chromosome 1 in the positive direction. The genomic start and stop positions of this connectron are 954.522 KB and 954.588 KB with a length of 0.067 KB. (4) The connectron that has the identifier 617 is on chromosome 1 in the negative direction. The genomic start and stop positions of this connectron are 954.658 KB and 954.616 KB with a length of 0.043 KB. (5) The connectron that has the identifier 618 is on chromosome 1 in the positive direction. The genomic start and stop positions of this connectron are 954.624 KB and 954.639 KB with a length of 0.016 KB. (6) The connectron that has the identifier 619 is on chromosome 1 in the positive direction. The genomic start and stop positions of this connectron are 954.641 KB and 954.658 KB with a length of 0.018 KB. (7) The connectron that has the identifier 622 is on chromosome 1 in the positive direction. The genomic start and stop positions of this connectron are 956.873 KB and 956.887 KB with a length of 0.015 KB. (8) The connectron that has the identifier 623 is on chromosome 1 in the positive direction. The genomic start and stop positions of this connectron are 956.896 KB and 956.910 KB with a length of 0.015 KB. (9) The connectron that has the identifier 629 is on chromosome 1 in the positive direction. The genomic start and stop positions of this connectron are 973.339 KB and 973.434 KB with a length of 0.096 KB. (10) The connectron that has the identifier 630 is on chromosome 1 in the positive direction. The genomic start and stop positions of this connectron are 973.508 KB and 973.603 KB with a length of 0.096 KB. (11) The connectron that has the identifier 634 is on chromosome 1 in the positive direction. The genomic start and stop positions of this connectron are 977.711 KB and 977.725 KB with a length of 0.015 KB. (12) The connectron that has the identifier 635 is on chromosome 1 in the positive direction. The genomic start and stop positions of this connectron are 977.732 KB and 977.746 KB with a length of 0.015 KB. (13) The connectron that has the identifier 637 is on chromosome 1 in the positive direction. The genomic start and stop positions of this connectron are 986.862 KB and 986.877 KB with a length of 0.016 KB. (14) The connectron that has the identifier 638 is on chromosome 1 in the positive direction. The genomic start and stop positions of this connectron are 986.885 KB and 986.899 KB with a length of 0.015 KB. (15) The connectron that has the identifier 644 is on chromosome 1 in the negative direction. The genomic start and stop positions of this connectron are 1005.921 KB and 1005.810 KB with a length of 0.112 KB. (16) The connectron that has the identifier 645 is on chromosome 1 in the positive direction. The genomic start and stop positions of this connectron are 1005.810 KB and 1005.926 KB with a length of 0.117 KB.

### Synthetic Connectron Formed by a PNA

A synthetic connectron can be introduced into the V. cholerae genome by introducing a PNA that recognizes the sequence

tccccatgag catagatatg caggtaggcg gcaagt

Connectron 7581 is an example of a synthetic transient connectron.

| | | C1/C2 | | | T1-T2 | | |
|---|---|---|---|---|---|---|---|
| Global_Id | Chromosome | C1_Id | C2_Id | Chromosome | T1_Id | T2_Id | Connectron_Type |
| 7581 | 1 | Synthetic | PNA | 1 | 607 | 646 | synthetic |

The T1-T2 target of the transient connectron 7581 as shown in Table 1 is represented as

| | | | | | | | |
|---|---|---|---|---|---|---|---|
| Type | Num | Jobno | Chr | Start | Stop | Length | GeneName |

| | | | | | | | |
|---|---|---|---|---|---|---|---|
| TN | 607 | 1 | 1 | 952.642 | 952.777 | .136 | *−++++*+++*+++++++*+*++++**++++ |
| TP | 608 | 1 | 1 | 952.648 | 952.777 | .130 | *−++++−+++−*+++*+*−++*++++−++++ |
| GG | 609 | 1 | 1 | 952.846 | 954.393 | 1.548 | Group0127 \|\|\|\| \|\|\| \|\|\| \| \|\| \|\|\|\| \|\|\|\| |
| TN | 610 | 1 | 1 | 954.486 | 954.587 | .102 | *−++++*+++*−+++−+−*+*−++*+**+++ |
| TP | 611 | 1 | 1 | 954.486 | 954.587 | .102 | *−+++++++++**++*+*++−**+−++−+*+ |
| CNT | 612 | 1 | 1 | 954.497 | 954.589 | .093 | OS−> \|\|\|\|\|\|\|\|\|\| \|\|\|\|\|\|\| \| \| \|\| \| \| |
| CPT | 613 | 1 | 1 | 954.503 | 954.520 | .018 | −−> \|\|\|\|\|\|\|\|\|\| \|\|\|\|\|\|\| \| \| \|\| \| \| |
| CPT | 614 | 1 | 1 | 954.522 | 954.588 | .067 | −−> \|\|\|\|\|\|\|\|\|\| \|\|\|\|\|\|\| \| \| \|\| \| \| |
| TP | 615 | 1 | 1 | 954.601 | 954.658 | .058 | *−++++++++++*+++++++*+*+*++*++−+−* |
| TN | 616 | 1 | 1 | 954.602 | 954.658 | .057 | *−+++++++++++++++++*+++++*+***− |
| CNT | 617 | 1 | 1 | 954.616 | 954.658 | .043 | −−> \|\|\|\|\|\|\|\|\|\|\|\|\|\|\|\|\| \|\|\|\|\| \|\| \| |
| CPT | 618 | 1 | 1 | 954.624 | 954.639 | .016 | −−> \|\|\|\|\|\|\|\|\|\|\|\|\|\|\|\|\| \|\|\|\|\| \|\| \| |
| CPT | 619 | 1 | 1 | 954.641 | 954.658 | .018 | −−> \|\|\|\|\|\|\|\|\|\|\|\|\|\|\|\|\| \|\|\|\|\| \|\| \| |
| GG | 620 | 1 | 1 | 954.684 | 958.635 | 3.951 | Group0128 \|\|\|\|\|\|\|\|\|\|\|\|\|\|\|\|\| \|\|\|\|\| \|\| \| |
| GP | 621 | 1 | 1 | 955.956 | 956.342 | .387 | VC0897 \|\|\|\|\|\|\|\|\|\|\|\|\|\|\|\|\| \|\|\|\|\| \|\| \| |
| CPT | 622 | 1 | 1 | 956.873 | 956.887 | .015 | −−> \|\|\|\|\|\|\|\|\|\|\|\|\|\|\|\|\| \|\|\|\|\| \|\| \| |
| CPT | 623 | 1 | 1 | 956.896 | 956.910 | .015 | −−> \|\|\|\|\|\|\|\|\|\|\|\|\|\|\|\|\| \|\|\|\|\| \|\| \| |
| TN | 624 | 1 | 1 | 958.089 | 958.103 | .015 | *−+*\|\|\|\|\|\|\|\|\|\|\|\|\|\|\| \|\|\|\|\| \|\| \| |
| GG | 625 | 1 | 1 | 958.840 | 972.992 | 14.152 | Group0129 \| \|\|\|\|\|\|\|\|\|\|\|\|\|\|\| \|\|\|\|\| \|\| \| |
| GP | 626 | 1 | 1 | 971.304 | 972.992 | 1.689 | VC0911 \| \|\|\|\|\|\|\|\|\|\|\|\|\|\|\| \|\|\|\|\| \|\| \| |
| TP | 627 | 1 | 1 | 973.170 | 973.250 | .081 | *−+*\|\|\|\|\|\|\|\|\|\|\|\|\|\|\| \|\|\|\|\| \|\| \| |
| GG | 628 | 1 | 1 | 973.171 | 973.763 | .592 | Group0130 \|\|\|\|\|\|\|\|\|\|\|\|\|\|\|\|\| \|\|\|\|\| \|\| \| |
| CPT | 629 | 1 | 1 | 973.339 | 973.434 | .096 | OS−> \|\|\|\|\|\|\|\|\|\|\|\|\|\|\|\|\| \|\|\|\|\| \|\| \| |
| CPT | 630 | 1 | 1 | 973.508 | 973.603 | .096 | OS−> \|\|\|\|\|\|\|\|\|\|\|\|\|\|\|\|\| \|\|\|\|\| \|\| \| |
| TP | 631 | 1 | 1 | 973.693 | 973.773 | .081 | *−+*\|\|\|\|\|\|\|\|\|\|\|\|\|\|\| \|\|\|\|\| \|\| \| |
| GG | 632 | 1 | 1 | 973.767 | 996.501 | 22.734 | Group0131 \| \|\|\|\|\|\|\|\|\|\|\|\|\|\|\| \|\|\|\|\| \|\| \| |
| GP | 633 | 1 | 1 | 974.049 | 978.217 | 4.169 | VC0913 \| \|\|\|\|\|\|\|\|\|\|\|\|\|\|\| \|\|\|\|\| \|\| \| |
| CPT | 634 | 1 | 1 | 977.711 | 977.725 | .015 | −−> \| \|\|\|\|\|\|\|\|\|\|\|\|\|\|\| \|\|\|\|\| \|\| \| |
| CPT | 635 | 1 | 1 | 977.732 | 977.746 | .015 | −−> \| \|\|\|\|\|\|\|\|\|\|\|\|\|\|\| \|\|\|\|\| \|\| \| |
| GP | 636 | 1 | 1 | 986.166 | 990.921 | 4.756 | VC0923 \| \|\|\|\|\|\|\|\|\|\|\|\|\|\|\| \|\|\|\|\| \|\| \| |
| CPT | 637 | 1 | 1 | 986.862 | 986.877 | .016 | −−> \| \|\|\|\|\|\|\|\|\|\|\|\|\|\|\| \|\|\|\|\| \|\| \| |
| CPT | 638 | 1 | 1 | 986.885 | 986.899 | .015 | −−> \| \|\|\|\|\|\|\|\|\|\|\|\|\|\|\| \|\|\|\|\| \|\| \| |
| TN | 639 | 1 | 1 | 996.556 | 996.577 | .022 | *−+**\|\|\|\|\|\|\|\|\|\|\|\|\|\| \|\|\|\|\| \|\| \| |
| TP | 640 | 1 | 1 | 996.556 | 996.577 | .022 | *−++−+++*++++++++++*\|\|\|\|\| \|\| \| |
| GG | 641 | 1 | 1 | 996.656 | 1005.792 | 9.136 | Group0132 \|\| \|\|\| \|\|\|\|\|\|\|\|\|\|\|\|\|\|\|\| \|\| \| |
| GP | 642 | 1 | 1 | 1002.462 | 1005.792 | 3.331 | VC0937 \|\| \|\|\| \|\|\|\|\|\|\|\|\|\|\|\|\|\|\|\| \|\| \| |
| TP | 643 | 1 | 1 | 1005.810 | 1005.874 | .065 | *−++*+++*++*+*+++++++++++*++*+* |
| CNT | 644 | 1 | 1 | 1005.810 | 1005.921 | .112 | OS−> \|\|\|\|\|\|\|\|\| \| \|\|\|\|\|\|\|\|\|\|\|\|\|\|\|\|\| |
| CPT | 645 | 1 | 1 | 1005.810 | 1005.926 | .117 | −−> \|\|\|\|\|\|\|\|\| \| \|\|\|\|\|\|\|\|\|\|\|\|\|\|\|\|\| |
| TN | 646 | 1 | 1 | 1005.810 | 1005.926 | .117 | *−++++*++++*+*+++++++++*+++*+++ |

Group0127 through Group0132 are described in Table 2 as

| Gene_Name | COG_Id | | Chromosome | Direction | Start | Stop | Length |
|---|---|---|---|---|---|---|---|
| Group0127 | | | | | | | |
| VC0894 | COG0607 | P | 1 | positive | 952.846 | 954.393 | 1.548 |
| Group0128 | | | | | | | |
| VC0895 | | | 1 | negative | 954.684 | 954.860 | .177 |
| VC0896 | COG0583 | K | 1 | positive | 955.014 | 955.934 | .921 |
| VC0897 | COG2363 | S | 1 | positive | 955.956 | 956.342 | .387 |
| VC0898 | COG0258 | L | 1 | negative | 956.372 | 957.223 | .852 |
| VC0899 | COG1611 | R | 1 | negative | 957.262 | 958.635 | 1.374 |
| Group0129 | | | | | | | |
| VC0900 | COG2199 | T | 1 | negative | 958.840 | 960.414 | 1.575 |
| VC0901 | COG0457 | R | 1 | negative | 960.516 | 962.777 | 2.262 |
| VC0902 | COG0780 | R | 1 | negative | 962.792 | 963.655 | .864 |
| VC0903 | | | 1 | positive | 963.686 | 964.282 | .597 |
| VC0904 | | | 1 | positive | 964.284 | 965.060 | .777 |
| VC0905 | COG1464 | M | 1 | negative | 965.193 | 966.020 | .828 |
| VC0906 | COG2011 | R | 1 | negative | 966.039 | 967.740 | 1.702 |
| VC0908 | COG0241 | E | 1 | positive | 967.976 | 968.536 | .561 |
| VC0909 | COG1609 | K | 1 | positive | 968.601 | 969.551 | .951 |
| VC0910 | COG1263 | G | 1 | positive | 969.788 | 971.224 | 1.437 |
| VC0911 | COG0366 | G | 1 | positive | 971.304 | 972.992 | 1.689 |
| Group0130 | | | | | | | |
| tRNA-Tyr-2 | | | 1 | positive | 973.171 | 973.255 | .085 |
| tRNA-Tyr-3 | | | 1 | positive | 973.340 | 973.424 | .085 |
| tRNA-Tyr-4 | | | 1 | positive | 973.509 | 973.593 | .085 |
| tRNA-Tyr-5 | | | 1 | positive | 973.679 | 973.763 | .085 |
| Group0131 | | | | | | | |
| VC0912 | | | 1 | positive | 973.767 | 973.862 | .096 |
| VC0913 | COG0845 | Q | 1 | positive | 974.049 | 978.217 | 4.169 |
| VC0915 | | | 1 | positive | 978.272 | 978.382 | .111 |
| VC0916 | COG0394 | T | 1 | positive | 978.644 | 979.144 | .501 |
| VC0917 | COG0381 | M | 1 | positive | 979.457 | 980.575 | 1.119 |
| VC0918 | COG0677 | M | 1 | positive | 980.595 | 986.155 | 5.561 |
| VC0923 | COG1045 | E | 1 | positive | 986.166 | 990.921 | 4.756 |
| VC0928 | | | 1 | positive | 991.117 | 991.932 | .816 |
| VC0929 | | | 1 | negative | 992.138 | 993.364 | 1.227 |
| VC0930 | | | 1 | positive | 993.628 | 996.501 | 2.874 |
| Group0132 | | | | | | | |
| VC0931 | COG3307 | M | 1 | negative | 996.656 | 997.972 | 1.317 |
| VC0932 | | | 1 | negative | 998.148 | 998.752 | .605 |
| VC0934 | COG2148 | M | 1 | positive | 999.242 | 1000.639 | 1.398 |
| VC0935 | | | 1 | positive | 1000.710 | 1001.906 | 1.197 |
| VC0936 | COG1596 | M | 1 | positive | 1001.910 | 1002.437 | .528 |
| VC0937 | COG3206 | N | 1 | positive | 1002.462 | 1005.792 | 3.331 |

All of the data for the transient connectron 7581 are pulled together in the following table that is the “terse” description of the connectron.

| Connectron Relationships - | Global_Id | Type |
|---|---|---|
| | 7581 | synthetic |

Control Sequences - Synthetic PNA that
recognizes the sequence
tccccatgag catagatatg caggtaggcg gcaagt

| | | Chro- | | | | |
|---|---|---|---|---|---|---|
| Target | | mo- | | | | |
| Sequences - | Direction | some | | Start | Stop | Length |
| | | | T1_Id | | | |
| | negative | 1 | 607 | 952.777 | 952.642 | .136 |
| | | | T2_Id | | | |
| | | | 646 | 1005.926 | 1005.810 | .117 |

Controlled Genes

| Local_Id | Chromosome | Group | Name | COG_Id | Direction | Start | Stop | Length |
|---|---|---|---|---|---|---|---|---|
| 1 | 1 | Group0127 | VC0894 | COG0607 | positive | 952.846 | 954.393 | 1.548 |
| 2 | 1 | Group0128 | VC0895 | | positive | 954.684 | 958.635 | 3.951 |
| 3 | 1 | Group0128 | VC0896 | COG0583 | positive | 955.014 | 955.934 | .921 |
| 4 | 1 | Group0128 | VC0897 | COG2363 | positive | 955.956 | 956.342 | .387 |
| 5 | 1 | Group0128 | VC0898 | COG0258 | negative | 957.223 | 956.372 | .852 |
| 6 | 1 | Group0128 | VC0899 | COG1611 | negative | 958.635 | 957.262 | 1.374 |
| 7 | 1 | Group0129 | VC0900 | COG2199 | positive | 958.840 | 972.992 | 14.152 |
| 8 | 1 | Group0129 | VC0901 | COG0457 | negative | 962.777 | 960.516 | 2.262 |
| 9 | 1 | Group0129 | VC0902 | COG0780 | negative | 963.655 | 962.792 | .864 |
| 10 | 1 | Group0129 | VC0903 | | positive | 963.686 | 964.282 | .597 |
| 11 | 1 | Group0129 | VC0904 | | positive | 964.284 | 965.060 | .777 |
| 12 | 1 | Group0129 | VC0905 | COG1464 | negative | 966.020 | 965.193 | .828 |
| 13 | 1 | Group0129 | VC0906 | COG2011 | negative | 967.740 | 966.039 | 1.702 |
| 14 | 1 | Group0129 | VC0908 | COG0241 | positive | 967.976 | 968.536 | .561 |
| 15 | 1 | Group0129 | VC0909 | COG1609 | positive | 968.601 | 969.551 | .951 |
| 16 | 1 | Group0129 | VC0910 | COG1263 | positive | 969.788 | 971.224 | 1.437 |
| 17 | 1 | Group0129 | VC0911 | COG0366 | positive | 971.304 | 972.992 | 1.689 |
| 18 | 1 | Group0130 | tRNA-Tyr- | | positive | 973.171 | 973.763 | .592 |
| 19 | 1 | Group0131 | VC0912 | | positive | 973.767 | 996.501 | 22.734 |
| 20 | 1 | Group0131 | VC0913 | COG0845 | positive | 974.049 | 978.217 | 4.169 |
| 21 | 1 | Group0131 | VC0915 | | positive | 978.272 | 978.382 | .111 |
| 22 | 1 | Group0131 | VC0916 | COG0394 | positive | 978.644 | 979.144 | .501 |
| 23 | 1 | Group0131 | VC0917 | COG0381 | positive | 979.457 | 980.575 | 1.119 |
| 24 | 1 | Group0131 | VC0918 | COG0677 | positive | 980.595 | 986.155 | 5.561 |
| 25 | 1 | Group0131 | VC0923 | COG1045 | positive | 986.166 | 990.921 | 4.756 |
| 26 | 1 | Group0131 | VC0928 | | positive | 991.117 | 991.932 | .816 |
| 27 | 1 | Group0131 | VC0929 | | negative | 993.364 | 992.138 | 1.227 |
| 28 | 1 | Group0131 | VC0930 | | positive | 993.628 | 996.501 | 2.874 |
| 29 | 1 | Group0132 | VC0931 | COG3307 | positive | 996.656 | 1005.792 | 9.136 |
| 30 | 1 | Group0132 | VC0932 | | negative | 998.752 | 998.148 | .605 |
| 31 | 1 | Group0132 | VC0934 | COG2148 | positive | 999.242 | 1000.639 | 1.398 |
| 32 | 1 | Group0132 | VC0935 | | positive | 1000.710 | 1001.906 | 1.197 |
| 33 | 1 | Group0132 | VC0936 | COG1596 | positive | 1001.910 | 1002.437 | .528 |
| 34 | 1 | Group0132 | VC0937 | COG3206 | positive | 1002.462 | 1005.792 | 3.331 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 612 | negative | 954.589 | 954.497 | .093 |
| 2 | 1 | 613 | positive | 954.503 | 954.520 | .018 |
| 3 | 1 | 614 | positive | 954.522 | 954.588 | .067 |
| 4 | 1 | 617 | negative | 954.658 | 954.616 | .043 |
| 5 | 1 | 618 | positive | 954.624 | 954.639 | .016 |
| 6 | 1 | 619 | positive | 954.641 | 954.658 | .018 |
| 7 | 1 | 622 | positive | 956.873 | 956.887 | .015 |
| 8 | 1 | 623 | positive | 956.896 | 956.910 | .015 |
| 9 | 1 | 629 | positive | 973.339 | 973.434 | .096 |
| 10 | 1 | 630 | positive | 973.508 | 973.603 | .096 |
| 11 | 1 | 634 | positive | 977.711 | 977.725 | .015 |
| 12 | 1 | 635 | positive | 977.732 | 977.746 | .015 |
| 13 | 1 | 637 | positive | 986.862 | 986.877 | .016 |
| 14 | 1 | 638 | positive | 986.885 | 986.899 | .015 |
| 15 | 1 | 644 | negative | 1005.921 | 1005.810 | .112 |
| 16 | 1 | 645 | positive | 1005.810 | 1005.926 | .117 |

The verbose description of the synthetic connectron 7581 is:

In the Vibrio cholerae chromosome I, complete chromosome the synthetic connectron number 7580 is generated by a synthetic PNA that recognizes the sequence tccccatgagcatagatatgcaggtaggcggcaagt. This connectron causes stabilization of a loop of DNA. The target sequences (T1-T2) are on the negative strand of the genomic DNA on chromosome 1. The identifier number of the initiating target sequence (T1) is 607. The genomic start and stop positions of this initiating target sequence are 952.777 KB and 952.642 KB with a length of 0.136 KB. The identifier number of the terminating target sequence (T2) is 646. The genomic start and stop positions of this terminating target sequence are 1005.926 KB and 1005.810 KB with a length of 0.117 KB.

This connectron controls the modulation of the expression of the following gene(s): (1) The gene that has the name VC0894 and with a COG identifier of COG0607. The genomic start and stop positions of this gene are 952.846 KB and 954.393 KB with a length of 1.548 KB. (2) The gene that has the name VC0895. The genomic start and stop positions of this gene are 954.684 KB and 958.635 KB with a length of 3.951 KB. (3) The gene that has the name VC0896 and with a COG identifier of COG0583. The genomic start and stop positions of this gene are 955.014 KB and 955.934 KB with a length of 0.921 KB. (4) The gene that has the name VC0897 and with a COG identifier of COG2363. The genomic start and stop positions of this gene are 955.956 KB and 956.342 KB with a length of 0.387 KB. (5) The gene that has the name VC0898 and with a COG identifier of COG0258. The genomic start and stop positions of this gene are 957.223 KB and 956.372 KB with a length of 0.852 KB. (6) The gene that has the name VC0899 and with a COG identifier of COG1611. The genomic start and stop positions of this gene are 958.635 KB and 957.262 KB with a length of 1.374 KB. (7) The gene that has the name VC0900 and with a COG identifier of COG2199. The genomic start and stop positions of this gene are 958.840 KB and 972.992 KB with a length of 14.152 KB. (8) The gene that has the name VC0901 and with a COG identifier of COG0457. The genomic start and stop positions of this gene are 962.777 KB and 960.516 KB with a length of 2.262 KB. (9) The gene that has the name VC0902 and with a COG identifier of COG0780. The genomic start and stop positions of this gene are 963.655 KB and 962.792 KB with a length of 0.864 KB. (10) The gene that has the name VC0903. The genomic start and stop positions of this gene are 963.686 KB and 964.282 KB with a length of 0.597 KB. (11) The gene that has the name VC0904. The genomic start and stop positions of this gene are 964.284 KB and 965.060 KB with a length of 0.777 KB. (12) The gene that has the name VC0905 and with a COG identifier of COG1464. The genomic start and stop positions of this gene are 966.020 KB and 965.193 KB with a length of 0.828 KB. (13) The gene that has the name VC0906 and with a COG identifier of COG2011. The genomic start and stop positions of this gene are 967.740 KB and 966.039 KB with a length of 1.702 KB. (14) The gene that has the name VC0908 and with a COG identifier of COG0241. The genomic start and stop positions of this gene are 967.976 KB and 968.536 KB with a length of 0.561 KB. (15) The gene that has the name VC0909 and with a COG identifier of COG1609. The genomic start and stop positions of this gene are 968.601 KB and 969.551 KB with a length of 0.951 KB. (16) The gene that has the name VC0910 and with a COG identifier of COG1263. The genomic start and stop positions of this gene are 969.788 KB and 971.224 KB with a length of 1.437 KB. (17) The gene that has the name VC0911 and with a COG identifier of COG0366. The genomic start and stop positions of this gene are 971.304 KB and 972.992 KB with a length of 1.689 KB. (18) The gene that has the name tRNA-Tyr-. The genomic start and stop positions of this gene are 973.171 KB and 973.763 KB with a length of 0.592 KB. (19) The gene that has the name VC0912. The genomic start and stop positions of this gene are 973.767 KB and 996.501 KB with a length of 22.734 KB. (20) The gene that has the name VC0913 and with a COG identifier of COG0845. The genomic start and stop positions of this gene are 974.049 KB and 978.217 KB with a length of 4.169 KB. (21) The gene that has the name VC0915. The genomic start and stop positions of this gene are 978.272 KB and 978.382 KB with a length of 0.111 KB. (22) The gene that has the name VC0916 and with a COG identifier of COG0394. The genomic start and stop positions of this gene are 978.644 KB and 979.144 KB with a length of 0.501 KB. (23) The gene that has the name VC0917 and with a COG identifier of COG0381. The genomic start and stop positions of this gene are 979.457 KB and 980.575 KB with a length of 1.119 KB. (24) The gene that has the name VC0918 and with a COG identifier of COG0677. The genomic start and stop positions of this gene are 980.595 KB and 986.155 KB with a length of 5.561 KB. (25) The gene that has the name VC0923 and with a COG identifier of COG1045. The genomic start and stop positions of this gene are 986.166 KB and 990.921 KB with a length of 4.756 KB. (26) The gene that has the name VC0928. The genomic start and stop positions of this gene are 991.117 KB and 991.932 KB with a length of 0.816 KB. (27) The gene that has the name VC0929. The genomic start and stop positions of this gene are 993.364 KB and 992.138 KB with a length of 1.227 KB. (28) The gene that has the name VC0930. The genomic start and stop positions of this gene are 993.628 KB and 996.501 KB with a length of 2.874 KB. (29) The gene that has the name VC0931 and with a COG identifier of COG3307. The genomic start and stop positions of this gene are 996.656 KB and 1005.792 KB with a length of 9.136 KB. (30) The gene that has the name VC0932. The genomic start and stop positions of this gene are 998.752 KB and 998.148 KB with a length of 0.605 KB. (31) The gene that has the name VC0934 and with a COG identifier of COG2148. The genomic start and stop positions of this gene are 999.242 KB and 1000.639 KB with a length of 1.398 KB. (32) The gene that has the name VC0935. The genomic start and stop positions of this gene are 1000.710 KB and 1001.906 KB with a length of 1.197 KB. (33) The gene that has the name VC0936 and with a COG identifier of COG1596. The genomic start and stop positions of this gene are 1001.910 KB and 1002.437 KB with a length of 0.528 KB. (34) The gene that has the name VC0937 and with a COG identifier of COG3206. The genomic start and stop positions of this gene are 1002.462 KB and 1005.792 KB with a length of 3.331 KB.

This connectron controls the turning off of the expression of the following connectron(s): (1) The connectron that has the identifier 612 is on chromosome 1 in the negative direction. The genomic start and stop positions of this connectron are 954.589 KB and 954.497 KB with a length of 0.093 KB. (2) The connectron that has the identifier 613 is on chromosome 1 in the positive direction. The genomic start and stop positions of this connectron are 954.503 KB and 954.520 KB with a length of 0.018 KB. (3) The connectron that has the identifier 614 is on chromosome 1 in the positive direction. The genomic start and stop positions of this connectron are 954.522 KB and 954.588 KB with a length of 0.067 KB. (4) The connectron that has the identifier 617 is on chromosome 1 in the negative direction. The genomic start and stop positions of this connectron are 954.658 KB and 954.616 KB with a length of 0.043 KB. (5) The connectron that has the identifier 618 is on chromosome 1 in the positive direction. The genomic start and stop positions of this connectron are 954.624 KB and 954.639 KB with a length of 0.016 KB. (6) The connectron that has the identifier 619 is on chromosome 1 in the positive direction. The genomic start and stop positions of this connectron are 954.641 KB and 954.658 KB with a length of 0.018 KB. (7) The connectron that has the identifier 622 is on chromosome 1 in the positive direction. The genomic start and stop positions of this connectron are 956.873 KB and 956.887 KB with a length of 0.015 KB. (8) The connectron that has the identifier 623 is on chromosome 1 in the positive direction. The genomic start and stop positions of this connectron are 956.896 KB and 956.910 KB with a length of 0.015 KB. (9) The connectron that has the identifier 629 is on chromosome 1 in the positive direction. The genomic start and stop positions of this connectron are 973.339 KB and 973.434 KB with a length of 0.096 KB. (10) The connectron that has the identifier 630 is on chromosome 1 in the positive direction. The genomic start and stop positions of this connectron are 973.508 KB and 973.603 KB with a length of 0.096 KB. (11) The connectron that has the identifier 634 is on chromosome 1 in the positive direction. The genomic start and stop positions of this connectron are 977.711 KB and 977.725 KB with a length of 0.015 KB. (12) The connectron that has the identifier 635 is on chromosome 1 in the positive direction. The genomic start and stop positions of this connectron are 977.732 KB and 977.746 KB with a length of 0.015 KB. (13) The connectron that has the identifier 637 is on chromosome 1 in the positive direction. The genomic start and stop positions of this connectron are 986.862 KB and 986.877 KB with a length of 0.016 KB. (14) The connectron that has the identifier 638 is on chromosome 1 in the positive direction. The genomic start and stop positions of this connectron are 986.885 KB and 986.899 KB with a length of 0.015 KB. (15) The connectron that has the identifier 644 is on chromosome 1 in the negative direction. The genomic start and stop positions of this connectron are 1005.921 KB and 1005.810 KB with a length of 0.112 KB. (16) The connectron that has the identifier 645 is on chromosome 1 in the positive direction. The genomic start and stop positions of this connectron are 1005.810 KB and 1005.926 KB with a length of 0.117 KB.

### Synthetic Connectron Formed by a Pair of Linked DNA Binding Objects

A synthetic connectron can be introduced into the A. pernix genome by introducing a pair of linked DNA binding objects that recognize the sequences

G1 cccgacacaacctgc
and
G2 cccggggttcccgag

Connectron 917 is an example of a synthetic transient connectron.

| | Chro- | | | | | | |
|---|---|---|---|---|---|---|---|
| | mo- | C1/C2 | | | T1-T2 | | |
| Global_Id | some | C1_Id | C2_Id | Chromosome | T1_Id | T2_Id | Connectron_Type |
| 917 | 1 | G1 | G2 | 1 | 218 | 295 | synthetic |

The T1-T2 target of the transient connectron 917 as shown in Table 1 is represented as

| | | | | | | | |
|---|---|---|---|---|---|---|---|
| Type | Num | Jobno | Chr | Start | Stop | Length | GeneName |

| | | | | | | | | |
|---|---|---|---|---|---|---|---|---|
| TN | 218 | 1 | 1 | 284.008 | 284.071 | .064 | | *−+*+−++*\|\|\|\| \|\|\| \| |
| TP | 219 | 1 | 1 | 284.008 | 284.071 | .064 | | −+++*\|\| \|\|\|\| \|\|\| \| |
| CPT | 220 | 1 | 1 | 284.009 | 284.071 | .063 | --> | \|\|\|\|\|\| \|\|\|\| \|\|\| \| |
| CNT | 221 | 1 | 1 | 284.073 | 284.144 | .072 | --> | \|\|\|\|\|\| \|\|\|\| \|\|\| \| |
| CPT | 222 | 1 | 1 | 284.073 | 284.145 | .073 | --> | \|\|\|\|\|\| \|\|\|\| \|\|\| \| |
| TP | 223 | 1 | 1 | 284.073 | 284.160 | .088 | | *−++++++*++++*\|\|\| \| |
| TN | 224 | 1 | 1 | 284.073 | 284.251 | .179 | | *−+++++++++++++++**+* |
| CNT | 225 | 1 | 1 | 284.158 | 284.251 | .094 | --> | \|\|\|\|\|\|\|\|\|\|\|\|\|\|\|\|\|\|\| |
| CPT | 226 | 1 | 1 | 284.158 | 284.251 | .094 | --> | \|\|\|\|\|\|\|\|\|\|\|\|\|\|\|\|\|\|\| |
| TP | 227 | 1 | 1 | 284.162 | 284.251 | .090 | | *−+++++++++++++++++++** |
| CPT | 228 | 1 | 1 | 284.253 | 284.280 | .028 | --> | \|\|\|\|\|\|\|\|\|\|\|\|\|\|\|\|\|\|\|\|\| |
| TN | 229 | 1 | 1 | 284.253 | 284.305 | .053 | | *−+++++++++++++++++++++* |
| TP | 230 | 1 | 1 | 284.253 | 284.316 | .064 | | *−++++++++++++++++++++++** |
| CPT | 231 | 1 | 1 | 284.283 | 284.305 | .023 | --> | \|\|\|\|\|\|\|\|\|\|\|\|\|\|\|\|\|\|\|\|\|\|\|\| |
| GG | 232 | 1 | 1 | 284.391 | 287.510 | 3.120 | Group0053 | \|\|\|\|\|\|\|\|\|\|\|\|\|\|\|\|\|\|\|\|\|\|\|\| |
| TN | 233 | 1 | 1 | 286.094 | 286.108 | .015 | | *−++++++++++++++++++++++++* |
| GG | 234 | 1 | 1 | 287.521 | 291.639 | 4.118 | Group0054 | \|\|\|\|\|\|\|\|\|\|\|\|\|\|\|\|\|\|\|\|\|\|\|\|\| |
| TP | 235 | 1 | 1 | 288.611 | 288.625 | .015 | | *−+++++*\|\|\|\|\|\|\|\|\|\|\|\|\|\|\|\|\|\|\| |
| GG | 236 | 1 | 1 | 291.643 | 295.139 | 3.496 | Group0055 | \|\|\|\|\| \|\|\|\|\|\|\|\|\|\|\|\|\|\|\|\|\|\|\| |
| TP | 237 | 1 | 1 | 295.230 | 295.259 | .030 | | *−+++++*\|\|\|\|\|\|\|\|\|\|\|\|\|\|\|\|\|\|\| |
| GG | 238 | 1 | 1 | 295.231 | 293.529 | .298 | Group0056 | \|\|\|\|\|\|\|\|\|\|\|\|\|\|\|\|\|\|\|\|\|\|\|\|\| |
| GP | 239 | 1 | 1 | 295.342 | 295.518 | .177 | APES013 | \|\|\|\|\|\|\|\|\|\|\|\|\|\|\|\|\|\|\|\|\|\|\|\|\| |
| CPT | 240 | 1 | 1 | 295.415 | 295.444 | .030 | --> | \|\|\|\|\|\|\|\|\|\|\|\|\|\|\|\|\|\|\|\|\|\|\|\|\| |
| TP | 241 | 1 | 1 | 295.417 | 295.444 | .028 | | *−+++++++++*+++++++++++++++* |
| GG | 242 | 1 | 1 | 295.572 | 296.676 | 1.104 | Group0057 | \|\|\|\|\|\|\|\|\| \|\|\|\|\|\|\|\|\|\|\|\|\|\|\|\| |
| TN | 243 | 1 | 1 | 296.080 | 296.095 | .016 | | *−+++++++++*\|\|\|\|\|\|\|\|\|\|\|\|\|\|\|\| |
| TN | 244 | 1 | 1 | 296.913 | 296.927 | .015 | | *−++++++++++++++++++++++++++* |
| GG | 245 | 1 | 1 | 297.071 | 298.857 | 1.786 | Group0058 | \|\|\|\|\|\|\|\|\|\|\|\|\|\|\|\|\|\|\|\|\|\|\|\|\|\|\| |
| TP | 246 | 1 | 1 | 298.617 | 298.631 | .015 | | *−+++++++++++++++++++++++++++* |
| GG | 247 | 1 | 1 | 298.856 | 305.941 | 7.085 | Group0059 | \|\|\|\|\|\|\|\|\|\|\|\|\|\|\|\|\|\|\|\|\|\|\|\|\|\|\|\| |
| CNT | 248 | 1 | 1 | 300.742 | 300.757 | .016 | --> | \|\|\|\|\|\|\|\|\|\|\|\|\|\|\|\|\|\|\|\|\|\|\|\|\|\|\|\| |
| CNT | 249 | 1 | 1 | 300.767 | 300.781 | .015 | --> | \|\|\|\|\|\|\|\|\|\|\|\|\|\|\|\|\|\|\|\|\|\|\|\|\|\|\|\| |
| GN | 250 | 1 | 1 | 301.243 | 303.153 | 1.911 | APE0428 | \|\|\|\|\|\|\|\|\|\|\|\|\|\|\|\|\|\|\|\|\|\|\|\|\|\|\|\| |
| TN | 251 | 1 | 1 | 301.733 | 301.748 | .016 | | *−++++++++++++*+++++++++++++++* |
| GP | 252 | 1 | 1 | 305.005 | 305.611 | .607 | APE0434 | \|\|\|\|\|\|\|\|\|\|\|\| \|\|\|\|\|\|\|\|\|\|\|\|\|\|\|\| |
| CPT | 253 | 1 | 1 | 305.334 | 305.348 | .015 | --> | \|\|\|\|\|\|\|\|\|\|\|\| \|\|\|\|\|\|\|\|\|\|\|\|\|\|\|\| |
| CPT | 254 | 1 | 1 | 305.358 | 305.372 | .015 | --> | \|\|\|\|\|\|\|\|\|\|\|\| \|\|\|\|\|\|\|\|\|\|\|\|\|\|\|\| |
| CNT | 255 | 1 | 1 | 305.552 | 305.566 | .015 | --> | \|\|\|\|\|\|\|\|\|\|\|\| \|\|\|\|\|\|\|\|\|\|\|\|\|\|\|\| |
| TN | 256 | 1 | 1 | 305.562 | 305.576 | .015 | | *−++++++++++++*++++++++++++++++ |
| CNT | 257 | 1 | 1 | 305.568 | 305.582 | .015 | --> | \|\|\|\|\|\|\|\|\|\|\|\|\|\|\|\|\|\|\|\|\|\|\|\|\|\|\|\|\| |
| GG | 258 | 1 | 1 | 306.121 | 308.859 | 2.738 | Group0060 | \|\|\|\|\|\|\|\|\|\|\|\|\|\|\|\|\|\|\|\|\|\|\|\|\|\|\|\|\| |
| TP | 259 | 1 | 1 | 307.532 | 307.547 | .016 | | *−+++++++++++++++++++++++++++++ |
| TN | 260 | 1 | 1 | 307.814 | 307.828 | .015 | | *−+++++++++++++++++++++++++++++ |
| TN | 261 | 1 | 1 | 308.559 | 308.573 | .015 | | *−+++++++++++++++++++++++++++++ |
| GG | 262 | 1 | 1 | 308.989 | 312.190 | 3.201 | Group0061 | \|\|\|\|\|\|\|\|\|\|\|\|\|\|\|\|\|\|\|\|\|\|\|\|\|\|\|\|\| |
| TN | 263 | 1 | 1 | 311.735 | 311.750 | .016 | | *−+++++++++++++++++++++++++++++ |
| GG | 264 | 1 | 1 | 312.215 | 313.258 | 1.043 | Group0062 | \|\|\|\|\|\|\|\|\|\|\|\|\|\|\|\|\|\|\|\|\|\|\|\|\|\|\|\|\| |
| TP | 265 | 1 | 1 | 312.894 | 312.909 | .016 | | *−+++++++++++++++++++++++++++++ |
| GG | 266 | 1 | 1 | 313.378 | 315.728 | 2.350 | Group0063 | \|\|\|\|\|\|\|\|\|\|\|\|\|\|\|\|\|\|\|\|\|\|\|\|\|\|\|\|\| |
| TN | 267 | 1 | 1 | 314.051 | 314.065 | .015 | | *−+++++++++++++++++++++++++++++ |
| TN | 268 | 1 | 1 | 314.826 | 314.840 | .015 | | *−+++++++++++++++++++++++++++++ |
| TN | 269 | 1 | 1 | 315.511 | 315.525 | .015 | | *−+++++++++++++++++++++++++++++ |
| TP | 270 | 1 | 1 | 315.747 | 315.761 | .015 | | *−+++++++++++++++++++++++++++++ |
| GG | 271 | 1 | 1 | 315.792 | 318.996 | 3.204 | Group0064 | \|\|\|\|\|\|\|\|\|\|\|\|\|\|\|\|\|\|\|\|\|\|\|\|\|\|\|\|\| |
| GP | 272 | 1 | 1 | 317.638 | 318.996 | 1.359 | APE0457 | \|\|\|\|\|\|\|\|\|\|\|\|\|\|\|\|\|\|\|\|\|\|\|\|\|\|\|\|\| |
| ONT | 273 | 1 | 1 | 317.668 | 317.682 | .015 | --> | \|\|\|\|\|\|\|\|\|\|\|\|\|\|\|\|\|\|\|\|\|\|\|\|\|\|\|\|\| |
| TP | 274 | 1 | 1 | 317.689 | 317.703 | .015 | | *−+++++++++++++++++++++++++++++ |
| CNT | 275 | 1 | 1 | 317.690 | 317.704 | .015 | --> | \|\|\|\|\|\|\|\|\|\|\|\|\|\|\|\|\|\|\|\|\|\|\|\|\|\|\|\|\| |
| GG | 276 | 1 | 1 | 318.432 | 318.914 | .483 | Group0065 | \|\|\|\|\|\|\|\|\|\|\|\|\|\|\|\|\|\|\|\|\|\|\|\|\|\|\|\|\| |
| CPT | 277 | 1 | 1 | 319.097 | 319.127 | .031 | --> | \|\|\|\|\|\|\|\|\|\|\|\|\|\|\|\|\|\|\|\|\|\|\|\|\|\|\|\|\| |
| TN | 278 | 1 | 1 | 319.122 | 319.137 | .016 | | *−+++++++++++++++++++++++++++++ |
| GG | 279 | 1 | 1 | 319.335 | 319.673 | .339 | Group0066 | \|\|\|\|\|\|\|\|\|\|\|\|\|\|\|\|\|\|\|\|\|\|\|\|\|\|\|\|\| |
| TN | 280 | 1 | 1 | 319.515 | 319.529 | .015 | | *−+++++++++++++++++++++++++++++ |
| TP | 281 | 1 | 1 | 319.572 | 319.586 | .015 | | *−+++++++++++++++++++++++++++++ |
| GG | 282 | 1 | 1 | 319.733 | 324.526 | 4.793 | Group0067 | \|\|\|\|\|\|\|\|\|\|\|\|\|\|\|\|\|\|\|\|\|\|\|\|\|\|\|\|\| |
| CNT | 283 | 1 | 1 | 320.484 | 320.513 | .030 | --> | \|\|\|\|\|\|\|\|\|\|\|\|\|\|\|\|\|\|\|\|\|\|\|\|\|\|\|\|\| |
| GN | 284 | 1 | 1 | 320.700 | 322.241 | 1.542 | APE0462 | \|\|\|\|\|\|\|\|\|\|\|\|\|\|\|\|\|\|\|\|\|\|\|\|\|\|\|\|\| |
| TN | 285 | 1 | 1 | 323.674 | 323.688 | .015 | | *−++++++++*\|\|\|\|\|\|\|\|\|\|\|\|\|\|\|\|\|\|\|\| |
| GG | 286 | 1 | 1 | 324.030 | 324.335 | .306 | Group0068 | \|\|\|\|\|\|\|\| \|\|\|\|\|\|\|\|\|\|\|\|\|\|\|\|\|\|\|\| |
| TN | 287 | 1 | 1 | 324.137 | 324.151 | .015 | | *−++++++++−++++++++++++++++++++ |
| GG | 288 | 1 | 1 | 324.606 | 325.575 | .969 | Group0069 | \|\|\|\|\|\|\|\| \|\|\|\|\|\|\|\|\|\|\|\|\|\|\|\|\|\|\|\| |
| GP | 289 | 1 | 1 | 325.141 | 325.575 | .435 | APE0469 | \|\|\|\|\|\|\|\| \|\|\|\|\|\|\|\|\|\|\|\|\|\|\|\|\|\|\|\| |
| CNT | 290 | 1 | 1 | 325.950 | 325.979 | .030 | --> | \|\|\|\|\|\|\|\| \|\|\|\|\|\|\|\|\|\|\|\|\|\|\|\|\|\|\|\| |
| CPT | 291 | 1 | 1 | 325.950 | 325.979 | .030 | --> | \|\|\|\|\|\|\|\| \|\|\|\|\|\|\|\|\|\|\|\|\|\|\|\|\|\|\|\| |
| TN | 292 | 1 | 1 | 325.959 | 325.975 | .017 | | *−++++++++−+*++++++++++++++++++ |
| GG | 293 | 1 | 1 | 325.980 | 326.717 | .738 | Group0070 | \|\|\|\|\|\|\|\| \| \|\|\|\|\|\|\|\|\|\|\|\|\|\|\|\|\|\| |
| CNT | 294 | 1 | 1 | 326.716 | 326.878 | .163 | OS-> | \|\|\|\|\|\|\|\| \| \|\|\|\|\|\|\|\|\|\|\|\|\|\|\|\|\|\| |
| TN | 295 | 1 | 1 | 326.716 | 326.878 | .163 | | *−+*+++++*−+−++++*\|\|\|\|\|\|\|\|\|\|\|\|\| |

The “Type” descriptor of the T1 (Id number 218) is “TN” showing that is T1 target is on the negative strand of the double-stranded genomic DNA. Because the T1 and the T2 targets have to be on the same strand, the T2 target (Id number 295) also has the type descriptor “TN”.

Group0053 through Group0070 are described in Table 2 as

| Gene_Name | COG_Id | | Chromosome | Direction | Start | Stop | Length |
|---|---|---|---|---|---|---|---|
| Group0053 | | | | | | | |
| APE0413 | COG0553 | K | 1 | negative | 284.391 | 287.510 | 3.120 |
| Group0054 | | | | | | | |
| APE0414 | | | 1 | negative | 287.521 | 288.066 | .546 |
| APE0415 | COG1483 | R | 1 | negative | 288.073 | 291.639 | 3.567 |
| Group0055 | | | | | | | |
| APE0416 | COG1743 | L | 1 | negative | 291.643 | 294.666 | 3.024 |
| APE0417 | | | 1 | positive | 292.319 | 292.636 | .318 |
| APE0418 | | | 1 | positive | 294.690 | 295.139 | .450 |
| Group0056 | | | | | | | |
| tRNA-Thr | | | 1 | positive | 295.231 | 295.325 | .095 |
| APES013 | | | 1 | positive | 295.342 | 295.518 | .177 |
| tRNA-Met | | | 1 | positive | 295.405 | 295.529 | .125 |
| Group0057 | | | | | | | |
| APES014 | | | 1 | negative | 295.572 | 295.754 | .183 |
| APE0419 | | | 1 | negative | 295.891 | 296.676 | .786 |
| Group0058 | | | | | | | |
| APE0420 | | | 1 | positive | 297.071 | 298.113 | 1.043 |
| APE0422 | | | 1 | negative | 297.448 | 297.993 | .546 |
| APE0423 | COG0084 | L | 1 | negative | 298.099 | 298.857 | .759 |
| Group0059 | | | | | | | |
| APE0424 | | | 1 | positive | 298.856 | 299.269 | .414 |
| APE0425 | | | 1 | negative | 298.973 | 299.332 | .360 |
| APE0426 | COG2246 | S | 1 | positive | 299.481 | 300.602 | 1.122 |
| APE0427 | COG1573 | L | 1 | positive | 300.649 | 301.296 | .648 |
| APE0428 | COG1750 | R | 1 | negative | 301.243 | 303.153 | 1.911 |
| APE0429 | | | 1 | positive | 303.244 | 303.786 | .543 |
| APE0430 | | | 1 | negative | 303.403 | 303.720 | .318 |
| APE0431 | | | 1 | positive | 303.833 | 304.911 | 1.079 |
| APE0432 | | | 1 | negative | 304.284 | 304.688 | .405 |
| APE0434 | | | 1 | positive | 305.005 | 305.611 | .607 |
| APE0436 | COG1093 | J | 1 | negative | 305.192 | 305.941 | .750 |
| Group0060 | | | | | | | |
| APES015 | COG2051 | J | 1 | negative | 306.121 | 306.342 | .222 |
| APES016 | COG1631 | J | 1 | negative | 306.359 | 306.550 | .192 |
| APE0437 | | | 1 | negative | 306.674 | 308.859 | 2.186 |
| APE0439 | | | 1 | positive | 307.209 | 307.841 | .633 |
| Group0061 | | | | | | | |
| APES017 | | | 1 | negative | 308.989 | 309.186 | .198 |
| APE0442 | COG1594 | K | 1 | negative | 309.201 | 309.515 | .315 |
| APES018 | | | 1 | negative | 309.642 | 309.842 | .201 |
| APE0443 | COG1761 | K | 1 | negative | 309.915 | 310.223 | .309 |
| APE0444 | COG3286 | S | 1 | negative | 310.227 | 310.832 | .606 |
| APE0445 | COG1096 | J | 1 | negative | 310.912 | 312.190 | 1.279 |
| Group0062 | | | | | | | |
| APE0447 | COG1736 | J | 1 | negative | 312.215 | 313.237 | 1.023 |
| APE0448 | | | 1 | positive | 312.650 | 313.258 | .609 |
| Group0063 | | | | | | | |
| APE0449 | COG0197 | J | 1 | negative | 313.378 | 313.902 | .525 |
| APE0450 | COG0303 | H | 1 | positive | 313.995 | 315.728 | 1.734 |
| APE0451 | | | 1 | negative | 314.028 | 314.714 | .687 |
| Group0064 | | | | | | | |
| APES019 | | | 1 | negative | 315.792 | 315.986 | .195 |
| APE0452 | | | 1 | negative | 316.237 | 316.791 | .555 |
| APE0453 | | | 1 | positive | 316.247 | 317.576 | 1.330 |
| APE0454 | COG1489 | G | 1 | negative | 316.820 | 317.542 | .723 |
| APE0456 | | | 1 | negative | 317.632 | 318.099 | .468 |
| APE0457 | COG0160 | E | 1 | positive | 317.638 | 318.996 | 1.359 |
| Group0065 | | | | | | | |
| APE0458 | | | 1 | negative | 318.432 | 318.914 | .483 |
| Group0066 | | | | | | | |
| APE0459 | | | 1 | negative | 319.335 | 319.673 | .339 |
| Group0067 | | | | | | | |
| APE0460 | | | 1 | positive | 319.733 | 320.038 | .306 |
| APE0461 | COG1591 | L | 1 | positive | 320.272 | 320.703 | .432 |
| APE0462 | COG1855 | R | 1 | negative | 320.700 | 322.241 | 1.542 |
| APE0463 | | | 1 | positive | 321.610 | 322.074 | .465 |
| APE0464 | | | 1 | positive | 322.317 | 323.228 | .912 |
| APE0465 | COG0301 | H | 1 | negative | 323.288 | 324.526 | 1.239 |
| Group0068 | | | | | | | |
| APE0466 | | | 1 | positive | 324.030 | 324.335 | .306 |
| Group0069 | | | | | | | |
| APE0468 | | | 1 | positive | 324.606 | 325.049 | .444 |
| APE0469 | COG1318 | K | 1 | positive | 325.141 | 325.575 | .435 |
| Group0070 | | | | | | | |
| APE0470 | COG1277 | R | 1 | negative | 325.980 | 326.717 | .738 |

All of the data for the transient connectron 917 are pulled together in the following table that is the “terse” description of the connectron.

| Connectron Relationships - | Global_Id | Type |
|---|---|---|
| | 917 | synthetic |

Control Sequences - Synthetic Pair of Linked
DNA Binding Objects that recognize the sequences
G1 = cccgacacaacctgc and G2 = cccggggttcccgag

| | | Chro- | | | | |
|---|---|---|---|---|---|---|
| Target | | mo- | | | | |
| Sequences - | Direction | some | | Start | Stop | Length |
| | | | T1_Id | | | |
| | negative | 1 | 218 | 284.071 | 284.008 | .064 |
| | | | T2_Id | | | |
| | | | 295 | 326.878 | 326.716 | .163 |

Controlled Genes

| Local_Id | Chromosome | Group | Name | COG_Id | Direction | Start | Stop | Length |
|---|---|---|---|---|---|---|---|---|
| 1 | 1 | Group0053 | APE0413 | COG0553 | positive | 284.391 | 287.510 | 3.120 |
| 2 | 1 | Group0054 | APE0414 | | positive | 287.521 | 291.639 | 4.118 |
| 3 | 1 | Group0054 | APE0415 | COG1483 | negative | 291.639 | 288.073 | 3.567 |
| 4 | 1 | Group0055 | APE0416 | COG1743 | positive | 291.643 | 295.139 | 3.496 |
| 5 | 1 | Group0055 | APE0417 | | positive | 292.319 | 292.636 | .318 |
| 6 | 1 | Group0055 | APE0418 | | positive | 294.690 | 295.139 | .450 |
| 7 | 1 | Group0056 | tRNA-Thr | | positive | 295.231 | 295.529 | .298 |
| 8 | 1 | Group0056 | APES013 | | positive | 295.342 | 295.518 | .177 |
| 9 | 1 | Group0057 | APES014 | | positive | 295.572 | 296.676 | 1.104 |
| 10 | 1 | Group0057 | APE0419 | | negative | 296.676 | 295.891 | .786 |
| 11 | 1 | Group0058 | APE0420 | | positive | 297.071 | 298.857 | 1.786 |
| 12 | 1 | Group0058 | APE0422 | | negative | 297.993 | 297.448 | .546 |
| 13 | 1 | Group0058 | APE0423 | COG0084 | negative | 298.857 | 298.099 | .759 |
| 14 | 1 | Group0059 | APE0424 | | positive | 298.856 | 305.941 | 7.085 |
| 15 | 1 | Group0059 | APE0425 | | negative | 299.332 | 298.973 | .360 |
| 16 | 1 | Group0059 | APE0426 | COG2246 | positive | 299.481 | 300.602 | 1.122 |
| 17 | 1 | Group0059 | APE0427 | COG1573 | positive | 300.649 | 301.296 | .648 |
| 18 | 1 | Group0059 | APE0428 | COG1750 | negative | 303.153 | 301.243 | 1.911 |
| 19 | 1 | Group0059 | APE0429 | | positive | 303.244 | 303.786 | .543 |
| 20 | 1 | Group0059 | APE0430 | | negative | 303.720 | 303.403 | .318 |
| 21 | 1 | Group0059 | APE0431 | | positive | 303.833 | 304.911 | 1.079 |
| 22 | 1 | Group0059 | APE0432 | | negative | 304.688 | 304.284 | .405 |
| 23 | 1 | Group0059 | APE0434 | | positive | 305.005 | 305.611 | .607 |
| 24 | 1 | Group0059 | APE0436 | COG1093 | negative | 305.941 | 305.192 | .750 |
| 25 | 1 | Group0060 | APES015 | COG2051 | positive | 306.121 | 308.859 | 2.738 |
| 26 | 1 | Group0060 | APES016 | COG1631 | negative | 306.550 | 306.359 | .192 |
| 27 | 1 | Group0060 | APE0437 | | negative | 308.859 | 306.674 | 2.186 |
| 28 | 1 | Group0060 | APE0439 | | positive | 307.209 | 307.841 | .633 |
| 29 | 1 | Group0061 | APES017 | | positive | 308.989 | 312.190 | 3.201 |
| 30 | 1 | Group0061 | APE0442 | COG1594 | negative | 309.515 | 309.201 | .315 |
| 31 | 1 | Group0061 | APES018 | | negative | 309.842 | 309.642 | .201 |
| 32 | 1 | Group0061 | APE0443 | COG1761 | negative | 310.223 | 309.915 | .309 |
| 33 | 1 | Group0061 | APE0444 | COG3286 | negative | 310.832 | 310.227 | .606 |
| 34 | 1 | Group0061 | APE0445 | COG1096 | negative | 312.190 | 310.912 | 1.279 |
| 35 | 1 | Group0062 | APE0447 | COG1736 | positive | 312.215 | 313.258 | 1.043 |
| 36 | 1 | Group0062 | APE0448 | | positive | 312.650 | 313.258 | .609 |
| 37 | 1 | Group0063 | APE0449 | COG0197 | positive | 313.378 | 315.728 | 2.350 |
| 38 | 1 | Group0063 | APE0450 | COG0303 | positive | 313.995 | 315.728 | 1.734 |
| 39 | 1 | Group0063 | APE0451 | | negative | 314.714 | 314.028 | .687 |
| 40 | 1 | Group0064 | APES019 | | positive | 315.792 | 318.996 | 3.204 |
| 41 | 1 | Group0064 | APE0452 | | negative | 316.791 | 316.237 | .555 |
| 42 | 1 | Group0064 | APE0453 | | positive | 316.247 | 317.576 | 1.330 |
| 43 | 1 | Group0064 | APE0454 | COG1489 | negative | 317.542 | 316.820 | .723 |
| 44 | 1 | Group0064 | APE0456 | | negative | 318.099 | 317.632 | .468 |
| 45 | 1 | Group0064 | APE0457 | COG0160 | positive | 317.638 | 318.996 | 1.359 |
| 46 | 1 | Group0065 | APE0458 | | positive | 318.432 | 318.914 | .483 |
| 47 | 1 | Group0066 | APE0459 | | positive | 319.335 | 319.673 | .339 |
| 48 | 1 | Group0067 | APE0460 | | positive | 319.733 | 324.526 | 4.793 |
| 49 | 1 | Group0067 | APE0461 | COG1591 | positive | 320.272 | 320.703 | .432 |
| 50 | 1 | Group0067 | APE0462 | COG1855 | negative | 322.241 | 320.700 | 1.542 |
| 51 | 1 | Group0067 | APE0463 | | positive | 321.610 | 322.074 | .465 |
| 52 | 1 | Group0067 | APE0464 | | positive | 322.317 | 323.228 | .912 |
| 53 | 1 | Group0067 | APE0465 | COG0301 | negative | 324.526 | 323.288 | 1.239 |
| 54 | 1 | Group0068 | APE0466 | | positive | 324.030 | 324.335 | .306 |
| 55 | 1 | Group0069 | APE0468 | | positive | 324.606 | 325.575 | .969 |
| 56 | 1 | Group0069 | APE0469 | COG1318 | positive | 325.141 | 325.575 | .435 |
| 57 | 1 | Group0070 | APE0470 | COG1277 | positive | 325.980 | 326.717 | .738 |

Controlled Connectrons

| | Chro- | | | | | |
|---|---|---|---|---|---|---|
| Local_{—} | mo- | C1/ | | | | |
| Id | some | C2_Id | Direction | Start | Stop | Length |
| 1 | 1 | 220 | positive | 284.009 | 284.071 | .063 |
| 2 | 1 | 221 | negative | 284.144 | 284.073 | .072 |
| 3 | 1 | 222 | positive | 284.073 | 284.145 | .073 |
| 4 | 1 | 225 | negative | 284.251 | 284.158 | .094 |
| 5 | 1 | 226 | positive | 284.158 | 284.251 | .094 |
| 6 | 1 | 228 | positive | 284.253 | 284.280 | .028 |
| 7 | 1 | 231 | positive | 284.283 | 284.305 | .023 |
| 8 | 1 | 240 | positive | 295.415 | 295.444 | .030 |
| 9 | 1 | 248 | negative | 300.757 | 300.742 | .016 |
| 10 | 1 | 249 | negative | 300.781 | 300.767 | .015 |
| 11 | 1 | 253 | positive | 305.334 | 305.348 | .015 |
| 12 | 1 | 254 | positive | 305.358 | 305.372 | .015 |
| 13 | 1 | 255 | negative | 305.566 | 305.552 | .015 |
| 14 | 1 | 257 | negative | 305.582 | 305.568 | .015 |
| 15 | 1 | 273 | negative | 317.682 | 317.668 | .015 |
| 16 | 1 | 275 | negative | 317.704 | 317.690 | .015 |
| 17 | 1 | 277 | positive | 319.097 | 319.127 | .031 |
| 18 | 1 | 283 | negative | 320.513 | 320.484 | .030 |
| 19 | 1 | 290 | negative | 325.979 | 325.950 | .030 |
| 20 | 1 | 291 | positive | 325.950 | 325.979 | .030 |
| 21 | 1 | 294 | negative | 326.878 | 326.716 | .163 |

The verbose description of the transient connectron 917 is:

In the Aeropyrum pernix K1 complete genome the synthetic connectron number 917 is generated by a synthetic pair of linked DNA binding objects that recognizes the sequences G1=cccgacacaacctgc and G2=cccggggttcccgag. The target sequences (T1-T2) are on the negative strand of the genomic DNA on chromosome 1. The identifier number of the initiating target sequence (T1) is 218. The genomic start and stop positions of this initiating target sequence are 284.071 KB and 284.008 KB with a length of 0.064 KB. The identifier number of the terminating target sequence (T2) is 295. The genomic start and stop positions of this terminating target sequence are 326.878 KB and 326.716 KB with a length of 0.163 KB.

This connectron controls the modulation of the expression of the following gene(s): (1) The gene that has the name APE0413 and with a COG identifier of COG0553. The genomic start and stop positions of this gene are 284.391 KB and 287.510 KB with a length of 3.120 KB. (2) The gene that has the name APE0414. The genomic start and stop positions of this gene are 287.521 KB and 291.639 KB with a length of 4.118 KB. (3) The gene that has the name APE0415 and with a COG identifier of COG1483. The genomic start and stop positions of this gene are 291.639 KB and 288.073 KB with a length of 3.567 KB. (4) The gene that has the name APE0416 and with a COG identifier of COG1743. The genomic start and stop positions of this gene are 291.643 KB and 295.139 KB with a length of 3.496 KB. (5) The gene that has the name APE0417. The genomic start and stop positions of this gene are 292.319 KB and 292.636 KB with a length of 0.318 KB. (6) The gene that has the name APE0418. The genomic start and stop positions of this gene are 294.690 KB and 295.139 KB with a length of 0.450 KB. (7) The gene that has the name tRNA-Thr. The genomic start and stop positions of this gene are 295.231 KB and 295.529 KB with a length of 0.298 KB. (8) The gene that has the name APES013. The genomic start and stop positions of this gene are 295.342 KB and 295.518 KB with a length of 0.177 KB. (9) The gene that has the name APES014. The genomic start and stop positions of this gene are 295.572 KB and 296.676 KB with a length of 1.104 KB. (10) The gene that has the name APE0419. The genomic start and stop positions of this gene are 296.676 KB and 295.891 KB with a length of 0.786 KB. (11) The gene that has the name APE0420. The genomic start and stop positions of this gene are 297.071 KB and 298.857 KB with a length of 1.786 KB. (12) The gene that has the name APE0422. The genomic start and stop positions of this gene are 297.993 KB and 297.448 KB with a length of 0.546 KB. (13) The gene that has the name APE0423 and with a COG identifier of COG0084. The genomic start and stop positions of this gene are 298.857 KB and 298.099 KB with a length of 0.759 KB. (14) The gene that has the name APE0424. The genomic start and stop positions of this gene are 298.856 KB and 305.941 KB with a length of 7.085 KB. (15) The gene that has the name APE0425. The genomic start and stop positions of this gene are 299.332 KB and 298.973 KB with a length of 0.360 KB. (16) The gene that has the name APE0426 and with a COG identifier of COG2246. The genomic start and stop positions of this gene are 299.481 KB and 300.602 KB with a length of 1.122 KB. (17) The gene that has the name APE0427 and with a COG identifier of COG1573. The genomic start and stop positions of this gene are 300.649 KB and 301.296 KB with a length of 0.648 KB. (18) The gene that has the name APE0428 and with a COG identifier of COG1750. The genomic start and stop positions of this gene are 303.153 KB and 301.243 KB with a length of 1.911 KB. (19) The gene that has the name APE0429. The genomic start and stop positions of this gene are 303.244 KB and 303.786 KB with a length of 0.543 KB. (20) The gene that has the name APE0430. The genomic start and stop positions of this gene are 303.720 KB and 303.403 KB with a length of 0.318 KB. (21) The gene that has the name APE0431. The genomic start and stop positions of this gene are 303.833 KB and 304.911 KB with a length of 1.079 KB. (22) The gene that has the name APE0432. The genomic start and stop positions of this gene are 304.688 KB and 304.284 KB with a length of 0.405 KB. (23) The gene that has the name APE0434. The genomic start and stop positions of this gene are 305.005 KB and 305.611 KB with a length of 0.607 KB. (24) The gene that has the name APE0436 and with a COG identifier of COG1093. The genomic start and stop positions of this gene are 305.941 KB and 305.192 KB with a length of 0.750 KB. (25) The gene that has the name APES015 and with a COG identifier of COG2051. The genomic start and stop positions of this gene are 306.121 KB and 308.859 KB with a length of 2.738 KB. (26) The gene that has the name APES016 and with a COG identifier of COG1631. The genomic start and stop positions of this gene are 306.550 KB and 306.359 KB with a length of 0.192 KB. (27) The gene that has the name APE0437. The genomic start and stop positions of this gene are 308.859 KB and 306.674 KB with a length of 2.186 KB. (28) The gene that has the name APE0439. The genomic start and stop positions of this gene are 307.209 KB and 307.841 KB with a length of 0.633 KB. (29) The gene that has the name APES017. The genomic start and stop positions of this gene are 308.989 KB and 312.190 KB with a length of 3.201 KB. (30) The gene that has the name APE0442 and with a COG identifier of COG1594. The genomic start and stop positions of this gene are 309.515 KB and 309.201 KB with a length of 0.315 KB. (31) The gene that has the name APES018. The genomic start and stop positions of this gene are 309.842 KB and 309.642 KB with a length of 0.201 KB. (32) The gene that has the name APE0443 and with a COG identifier of COG1761. The genomic start and stop positions of this gene are 310.223 KB and 309.915 KB with a length of 0.309 KB. (33) The gene that has the name APE0444 and with a COG identifier of COG3286. The genomic start and stop positions of this gene are 310.832 KB and 310.227 KB with a length of 0.606 KB. (34) The gene that has the name APE0445 and with a COG identifier of COG1096. The genomic start and stop positions of this gene are 312.190 KB and 310.912 KB with a length of 1.279 KB. (35) The gene that has the name APE0447 and with a COG identifier of COG1736. The genomic start and stop positions of this gene are 312.215 KB and 313.258 KB with a length of 1.043 KB. (36) The gene that has the name APE0448. The genomic start and stop positions of this gene are 312.650 KB and 313.258 KB with a length of 0.609 KB. (37) The gene that has the name APE0449 and with a COG identifier of COG0197. The genomic start and stop positions of this gene are 313.378 KB and 315.728 KB with a length of 2.350 KB. (38) The gene that has the name APE0450 and with a COG identifier of COG0303. The genomic start and stop positions of this gene are 313.995 KB and 315.728 KB with a length of 1.734 KB. (39) The gene that has the name APE0451. The genomic start and stop positions of this gene are 314.714 KB and 314.028 KB with a length of 0.687 KB. (40) The gene that has the name APES019. The genomic start and stop positions of this gene are 315.792 KB and 318.996 KB with a length of 3.204 KB. (41) The gene that has the name APE0452. The genomic start and stop positions of this gene are 316.791 KB and 316.237 KB with a length of 0.555 KB. (42) The gene that has the name APE0453. The genomic start and stop positions of this gene are 316.247 KB and 317.576 KB with a length of 1.330 KB. (43) The gene that has the name APE0454 and with a COG identifier of COG1489. The genomic start and stop positions of this gene are 317.542 KB and 316.820 KB with a length of 0.723 KB. (44) The gene that has the name APE0456. The genomic start and stop positions of this gene are 318.099 KB and 317.632 KB with a length of 0.468 KB. (45) The gene that has the name APE0457 and with a COG identifier of COG0160. The genomic start and stop positions of this gene are 317.638 KB and 318.996 KB with a length of 1.359 KB. (46) The gene that has the name APE0458. The genomic start and stop positions of this gene are 318.432 KB and 318.914 KB with a length of 0.483 KB. (47) The gene that has the name APE0459. The genomic start and stop positions of this gene are 319.335 KB and 319.673 KB with a length of 0.339 KB. (48) The gene that has the name APE0460. The genomic start and stop positions of this gene are 319.733 KB and 324.526 KB with a length of 4.793 KB. (49) The gene that has the name APE0461 and with a COG identifier of COG1591. The genomic start and stop positions of this gene are 320.272 KB and 320.703 KB with a length of 0.432 KB. (50) The gene that has the name APE0462 and with a COG identifier of COG1855. The genomic start and stop positions of this gene are 322.241 KB and 320.700 KB with a length of 1.542 KB. (51) The gene that has the name APE0463. The genomic start and stop positions of this gene are 321.610 KB and 322.074 KB with a length of 0.465 KB. (52) The gene that has the name APE0464. The genomic start and stop positions of this gene are 322.317 KB and 323.228 KB with a length of 0.912 KB. (53) The gene that has the name APE0465 and with a COG identifier of COG0301. The genomic start and stop positions of this gene are 324.526 KB and 323.288 KB with a length of 1.239 KB. (54) The gene that has the name APE0466. The genomic start and stop positions of this gene are 324.030 KB and 324.335 KB with a length of 0.306 KB. (55) The gene that has the name APE0468. The genomic start and stop positions of this gene are 324.606 KB and 325.575 KB with a length of 0.969 KB. (56) The gene that has the name APE0469 and with a COG identifier of COG1318. The genomic start and stop positions of this gene are 325.141 KB and 325.575 KB with a length of 0.435 KB. (57) The gene that has the name APE0470 and with a COG identifier of COG1277. The genomic start and stop positions of this gene are 325.980 KB and 326.717 KB with a length of 0.738 KB.

This connectron controls the turning off of the expression of the following connectron(s): (1) The connectron that has the identifier 220 is on chromosome 1 in the positive direction. The genomic start and stop positions of this connectron are 284.009 KB and 284.071 KB with a length of 0.063 KB. (2) The connectron that has the identifier 221 is on chromosome 1 in the negative direction. The genomic start and stop positions of this connectron are 284.144 KB and 284.073 KB with a length of 0.072 KB. (3) The connectron that has the identifier 222 is on chromosome 1 in the positive direction. The genomic start and stop positions of this connectron are 284.073 KB and 284.145 KB with a length of 0.073 KB. (4) The connectron that has the identifier 225 is on chromosome 1 in the negative direction. The genomic start and stop positions of this connectron are 284.251 KB and 284.158 KB with a length of 0.094 KB. (5) The connectron that has the identifier 226 is on chromosome 1 in the positive direction. The genomic start and stop positions of this connectron are 284.158 KB and 284.251 KB with a length of 0.094 KB. (6) The connectron that has the identifier 228 is on chromosome 1 in the positive direction. The genomic start and stop positions of this connectron are 284.253 KB and 284.280 KB with a length of 0.028 KB. (7) The connectron that has the identifier 231 is on chromosome 1 in the positive direction. The genomic start and stop positions of this connectron are 284.283 KB and 284.305 KB with a length of 0.023 KB. (8) The connectron that has the identifier 240 is on chromosome 1 in the positive direction. The genomic start and stop positions of this connectron are 295.415 KB and 295.444 KB with a length of 0.030 KB. (9) The connectron that has the identifier 248 is on chromosome 1 in the negative direction. The genomic start and stop positions of this connectron are 300.757 KB and 300.742 KB with a length of 0.016 KB. (10) The connectron that has the identifier 249 is on chromosome 1 in the negative direction. The genomic start and stop positions of this connectron are 300.781 KB and 300.767 KB with a length of 0.015 KB. (11) The connectron that has the identifier 253 is on chromosome 1 in the positive direction. The genomic start and stop positions of this connectron are 305.334 KB and 305.348 KB with a length of 0.015 KB. (12) The connectron that has the identifier 254 is on chromosome 1 in the positive direction. The genomic start and stop positions of this connectron are 305.358 KB and 305.372 KB with a length of 0.015 KB. (13) The connectron that has the identifier 255 is on chromosome 1 in the negative direction. The genomic start and stop positions of this connectron are 305.566 KB and 305.552 KB with a length of 0.015 KB. (14) The connectron that has the identifier 257 is on chromosome 1 in the negative direction. The genomic start and stop positions of this connectron are 305.582 KB and 305.568 KB with a length of 0.015 KB. (15) The connectron that has the identifier 273 is on chromosome 1 in the negative direction. The genomic start and stop positions of this connectron are 317.682 KB and 317.668 KB with a length of 0.015 KB. (16) The connectron that has the identifier 275 is on chromosome 1 in the negative direction. The genomic start and stop positions of this connectron are 317.704 KB and 317.690 KB with a length of 0.015 KB. (17) The connectron that has the identifier 277 is on chromosome 1 in the positive direction. The genomic start and stop positions of this connectron are 319.097 KB and 319.127 KB with a length of 0.031 KB. (18) The connectron that has the identifier 283 is on chromosome 1 in the negative direction. The genomic start and stop positions of this connectron are 320.513 KB and 320.484 KB with a length of 0.030 KB. (19) The connectron that has the identifier 290 is on chromosome 1 in the negative direction. The genomic start and stop positions of this connectron are 325.979 KB and 325.950 KB with a length of 0.030 KB. (20) The connectron that has the identifier 291 is on chromosome 1 in the positive direction. The genomic start and stop positions of this connectron are 325.950 KB and 325.979 KB with a length of 0.030 KB. (21) The connectron that has the identifier 294 is on chromosome 1 in the negative direction. The genomic start and stop positions of this connectron are 326.878 KB and 326.716 KB with a length of 0.163 KB.

1. The method of simulating connectron behavior of any genome for the purpose of modifying genomic behavior comprising the selective deletion and/or addition of connectrons.

2. The method of simulating connectron behavior of a genome for the purpose of modifying, by computer, genomic behavior comprising the selective deletion and/or addition of connectrons.

3. A genome having a connectron comprised of the DNA elements C1, C2, T1 and T2, wherein at least one of said DNA elements has been deleted according to the following table

| Type | C1 | C2 | T1 | T2 |
|---|---|---|---|---|
| 1 | D | | | |
| 2 | | D | | |
| 3 | | | D | |
| 4 | | | | D |
| 5 | D | D | | |
| 6 | D | | D | |
| 7 | D | | | D |
| 8 | D | D | D | |
| 9 | D | D | | D |
| 10 | D | D | D | D |

where D is a connectron element to be deleted or modified.

4. A genome having a connectron comprised of the DNA elements C1, C2, T1 and T2, wherein at least one of said DNA elements has been replaced with a synthetic DNA element.

5. A genome having a connectron comprised of the native DNA elements C1, C2, T1 and T2, wherein at least one of said DNA elements has been replaced with a synthetic DNA element according to the following table

| Type | C1 | C2 | T1 | T2 |
|---|---|---|---|---|
| 1 | S | S | S | S |
| 2 | S | S | S | N |
| 3 | S | S | N | S |
| 4 | S | S | N | N |
| 5 | S | N | S | S |
| 6 | S | N | S | N |
| 7 | S | N | N | S |
| 8 | S | N | N | N |
| 9 | N | S | S | S |
| 10 | N | S | S | N |
| 11 | N | S | N | S |
| 12 | N | S | N | N |
| 13 | N | N | S | S |
| 14 | N | N | S | N |
| 15 | N | N | N | S |

where N is native DNA sequence and S is a synthetic DNA sequence.

6. A genome having a connectron comprised of the native DNA elements T1 and T2, wherein none, one or both of said DNA elements have been replaced with a synthetic DNA element according to the following table and where the C1 and C2 sequences are generated by the binding of a synthetic DBP according to the following table

| Type | C1 | C2 | T1 | T2 |
|---|---|---|---|---|
| 1 | D | D | S | S |
| 2 | D | D | S | N |
| 3 | D | D | N | S |
| 4 | D | D | N | N |

where N is native DNA sequence, S is a synthetic DNA sequence and D is a synthetic DBP.

7. A genome having a connectron comprised of the native DNA elements T1 and T2, wherein none, one or both of said DNA elements have been replaced with a synthetic DNA element according to the following table and where the C1 and C2 sequences are generated by the binding of a synthetic PNA according to the following table

| Type | C1 | C2 | T1 | T2 |
|---|---|---|---|---|
| 1 | P | P | S | S |
| 2 | P | P | S | N |
| 3 | P | P | N | S |
| 4 | P | P | N | N |

where N is native DNA sequence, S is a synthetic DNA sequence and P is a synthetic PNA.

8. A genome having a connectron comprised of the native DNA elements T1 and T2, wherein none, one or both of said DNA elements has been replaced with a synthetic DNA element according to the following table and where the C1 and C2 sequences are generated by the binding a linked pair of DNA binding elements G1 and G2 according to the following table

| Type | C1 | C2 | T1 | T2 |
|---|---|---|---|---|
| 1 | G1 | G2 | S | S |
| 2 | G1 | G2 | S | N |
| 3 | G1 | G2 | N | S |
| 4 | G1 | G2 | N | N |

where N is native DNA sequence, S is a synthetic DNA sequence and where G1 and G2 are a linked pair of double-strand DNA binding elements.

9. A genome having a connectron comprised of the native DNA elements C1, C2, T1 and T2, wherein one of said DNA elements has been replaced with a synthetic DNA element according to the following table

| Type | Element-1-Style |
|---|---|
| 1 | C |
| 2 | E |

where C is a copied sequence element, E is an extracted sequence element.

10. A genome having a connectron comprised of the native DNA elements C1, C2, T1 and T2, wherein two of said DNA elements have been replaced with a synthetic DNA element according to the following table

| | |
|---|---|
| Element-1 | Element-2 |
| Type | Type | Style | Type | Style |
|---|---|---|---|---|
| 1 | C1 | C | C2 | C |
| 2 | C1 | C | C2 | E |
| 3 | C1 | E | C2 | E |
| 4 | C1 | C | T1 | C |
| 5 | C1 | C | T1 | E |
| 6 | C1 | U | T1 | U |
| 7 | C1 | E | T2 | E |
| 8 | C2 | C | T2 | C |
| 9 | C2 | C | T2 | E |
| 10 | C2 | U | T2 | U |
| 11 | C2 | E | T2 | E |
| 12 | T1 | C | T2 | C |
| 13 | T1 | C | T2 | E |
| 14 | T1 | E | T2 | E |

where C is a copied sequence element, U is a unique sequence element and E is an extracted sequence element.

11. A genome having a connectron comprised of the native DNA elements C1, C2, T1 and T2, wherein three of said DNA elements have been replaced with a synthetic DNA element according to the following table

| | | |
|---|---|---|
| Element-1 | Element-2 | Element-3 |
| Type | Type | Style | Type | Style | Type | Style |
|---|---|---|---|---|---|---|
| 1 | C1 | C | C2 | C | T1 | C |
| 2 | C1 | C | C2 | C | T1 | E |
| 3 | C1 | C | C2 | E | T1 | C |
| 4 | C1 | C | C2 | E | T1 | E |
| 5 | C1 | U | C2 | C | T1 | U |
| 6 | C1 | U | C2 | U | T1 | E |
| 7 | C1 | U | C2 | E | T1 | U |
| 8 | C1 | E | C2 | C | T1 | C |
| 9 | C1 | E | C2 | C | T1 | E |
| 10 | C1 | E | C2 | E | T1 | C |
| 11 | C1 | E | C2 | E | T1 | E |
| 12 | C1 | C | C2 | C | T2 | C |
| 13 | C1 | C | C2 | C | T2 | E |
| 14 | C1 | C | C2 | U | T2 | U |
| 15 | C1 | C | C2 | E | T2 | C |
| 16 | C1 | C | C2 | E | T2 | E |
| 17 | C1 | E | C2 | C | T2 | C |
| 18 | C1 | E | C2 | C | T2 | E |
| 19 | C1 | E | C2 | U | T2 | U |
| 20 | C1 | E | C2 | E | T2 | C |
| 21 | C1 | E | C2 | E | T2 | E |
| 22 | C1 | C | T1 | C | T2 | C |
| 23 | C1 | C | T1 | C | T2 | E |
| 24 | C1 | C | T1 | E | T2 | C |
| 25 | C1 | C | T1 | E | T2 | E |
| 26 | C1 | U | T1 | U | T2 | C |
| 27 | C1 | U | T1 | U | T2 | E |
| 28 | C1 | E | T1 | C | T2 | C |
| 29 | C1 | E | T1 | C | T2 | E |
| 30 | C1 | E | T1 | E | T2 | C |
| 31 | C1 | E | T1 | E | T2 | E |
| 32 | C2 | C | T1 | C | T2 | C |
| 33 | C2 | C | T1 | C | T2 | E |
| 34 | C2 | C | T1 | E | T2 | C |
| 35 | C2 | C | T1 | E | T2 | E |
| 36 | C2 | U | T1 | C | T2 | U |
| 37 | C2 | U | T1 | E | T2 | U |
| 38 | C2 | E | T1 | C | T2 | C |
| 39 | C2 | E | T1 | C | T2 | E |
| 40 | C2 | E | T1 | E | T2 | C |
| 41 | C2 | E | T1 | E | T2 | E |

where C is a copied sequence element, U is a unique sequence element and E is an extracted sequence element.

12. A genome having a connectron comprised of the native DNA elements C1, C2, T1 and T2, wherein four of said DNA elements have been replaced with a synthetic DNA element according to the following table

| | C1 | C2 | T1 | T2 |
|---|---|---|---|---|
| Type | Style | Style | Style | Style |
| 1 | C | C | C | C |
| 2 | C | C | C | E |
| 3 | C | C | E | C |
| 4 | C | C | E | E |
| 5 | C | U | C | U |
| 6 | C | U | E | U |
| 7 | C | E | C | C |
| 8 | C | E | C | E |
| 9 | C | E | E | C |
| 10 | C | E | E | E |
| 11 | U | C | U | C |
| 12 | U | C | U | E |
| 13 | U | U | U | U |
| 14 | U | E | U | C |
| 15 | U | E | U | E |
| 16 | E | C | C | C |
| 17 | E | C | C | E |
| 18 | E | C | E | C |
| 19 | E | C | E | E |
| 20 | E | U | C | U |
| 21 | E | U | E | U |
| 22 | E | E | C | C |
| 23 | E | E | C | E |
| 24 | E | E | E | C |
| 25 | E | E | E | E |

where C is a copied sequence element, U is a unique sequence element and E is an extracted sequence element.
